(19) United States
(12) Patent Application Publication
(10) Pub. No.: US 2014/0186827 A1
(43) Pub. Date: Jul. 3, 2014

(54) ASSAYS FOR THE DETECTION OF GENOTYPE, MUTATIONS, AND/OR ANEUPLOIDY

(71) Applicants: Martin Pieprzyk, Belmont, CA (US); Robert C. Jones, Los Altos, CA (US); Kenneth J. Livak, San Jose, CA (US); Andrew May, San Francisco, CA (US); Alain Mir, Cupertino, CA (US); Jian Qin, Foster City, CA (US); Ramesh Ramakrishnan, San Jose, CA (US); Sandra Spurgeon, San Mateo, CA (US); Jun Wang, Palo Alto, CA (US); Bernhard G. Zimmermann, San Mateo, CA (US)

(72) Inventors: Martin Pieprzyk, Belmont, CA (US); Robert C. Jones, Los Altos, CA (US); Kenneth J. Livak, San Jose, CA (US); Andrew May, San Francisco, CA (US); Alain Mir, Cupertino, CA (US); Jian Qin, Foster City, CA (US); Ramesh Ramakrishnan, San Jose, CA (US); Sandra Spurgeon, San Mateo, CA (US); Jun Wang, Palo Alto, CA (US); Bernhard G. Zimmermann, San Mateo, CA (US)

(73) Assignee: FLUIDIGM, INC., South San Francisco, CA (US)

(21) Appl. No.: 13/977,629

(22) PCT Filed: May 16, 2011
(86) PCT No.: PCT/US11/00887

§ 371 Date: Feb. 19, 2014

Related U.S. Application Data
(60) Provisional application No. 61/395,551, filed on May 14, 2010.

(51) Int. Cl. C12Q 1/68

(52) U.S. Cl. CPC C12Q 1/6851
USPC 435/6.11

(57) ABSTRACT

The present invention provides amplification-based methods for detection of genotype, mutations, and/or aneuploidy. These methods have broad applicability, but are particularly well-suited to detecting and quantifying target nucleic acids in free fetal DNA present in a maternal bodily fluid sample.

### CROSS-REFERENCE TO RELATED APPLICATIONS

This application claims the benefit of U.S. provisional application No. 61/395,551, filed May 14, 2010, which is hereby incorporated by reference in its entirety.

### FIELD OF THE INVENTION

The present invention relates to generally to the area of detecting genotype and/or aneuploidy. In particular, the invention relates to methods and compositions for detecting fetal genotype and/or aneuploidy in a maternal bodily fluid sample, such as blood or urine.

### BACKGROUND OF THE INVENTION

Cell-free fetal DNA is present in maternal bodily fluids from a pregnant woman, such blood. Detecting genotype (e.g., mutations) and/or aneuploidy in such fetal DNA in a maternal sample is difficult due to the presence of cell-free maternal DNA at a much higher percentage than the fetal DNA, which constitutes only about 5 percent, or less, of the total DNA in such samples. Similar difficulties exist with respect to the detection of cell-free tumor DNA in bodily fluids from cancer patients.

### SUMMARY OF THE INVENTION

A first method of the of the invention is a method for detecting and/or quantifying one or more target amplicon(s) produced by amplification, wherein the detecting and/or quantifying is carried out during amplification or after an amplification endpoint has been reached. The method entails the method including preparing an amplification reaction mixture including:
sample nucleic acids;
at least one target-specific primer pair;
an optional probe, wherein at least one primer of the target-specific primer pair or the probe, if present, is labeled with a fluorescent dye; and
a fluorescent double-stranded DNA-binding dye, where fluorescence from the dye is capable of quenching fluorescent signal from the labeled primer or probe, if present.
   The amplification mixture is subjected to amplification, and the fluorescent signal is detected to detect and/or quantify the target amplicon(s).

An embodiment of the first method entails preparing an amplification reaction mixture including:
sample nucleic acids;
at least one target-specific primer pair, wherein at least one primer in the target-specific primer pair comprises a nucleotide tag at the 5′ end of the primer;
at least one fluorescently labeled primer or probe that is capable of annealing to the nucleotide tag, directly or via one or more intervening primers, whereby the label can become linked to the nucleotide tag; and
a fluorescent double-stranded DNA-binding dye, where fluorescence from the dye is capable of quenching fluorescent signal from the labeled primer or probe.
   The amplification mixture is subjected to amplification, and the fluorescent signal is detected to detect and/or quantify the target amplicon(s). In a variation of this embodiment, the fluorescence from the dye quenches fluorescent signal from the labeled primer or probe when the labeled primer or probe is incorporated into, or hybridized to, an amplification product.

A second method of the invention is a method for detecting an allele in a sample. The method entails preparing an amplification mixture including:
sample nucleic acids;
two allele-specific primer pairs, wherein:
   at least one primer in each primer pair is specific for an allele and is tagged with a distinct nucleotide tag at the 5′ end of the primer; and
   the other primer in each pair can be the same or different from one another;
at least two differently fluorescently labeled primers or probes, each capable of annealing to one of the nucleotide tags, directly or via one or more intervening primers, whereby one label can become linked to one nucleotide tag and a different label can become linked to the other nucleotide tag.
   The amplification mixture is then subjected to amplification, and the fluorescent signal is detected to detect the allele in the sample.

In certain embodiments of the second method, the amplification mixture additionally includes a fluorescent double-stranded DNA-binding dye, wherein fluorescence from the dye is capable of quenching fluorescent signal from the labeled primers or probes. In illustrative embodiments, the fluorescence from the dye quenches fluorescent signal from the labeled primers or probes when the labeled primers or probes are incorporated into, or hybridized to, an amplification product.

In particular embodiments of the second method, two differently labeled primers are employed, and the method additionally entails including in the reaction one or more quencher oligonucleotide(s) that include(s) a sequence that is capable of hybridizing to at least part of the nucleotide tag(s) and a fluorescence quencher, wherein hybridization to unincorporated fluorescently labeled primer(s) quenches the fluorescent label(s). In variations of such embodiments, the fluorescence quencher is at the 3′ end of the quencher oligonucleotide or is attached to an internal nucleotide of the quencher oligonucleotide. In specific embodiments, the amplification mixture includes at least two quencher oligonucleotides, one specific for each nucleotide tag.

A third method of the invention is another method for detecting an allele in a sample. The method entails preparing an amplification mixture including:
sample nucleic acids;
two allele-specific oligonucleotides, wherein each oligonucleotide includes a target-specific sequence linked to a distinct 3′ nucleotide tag; and
at least two differently fluorescently labeled primers or probes, each capable of annealing to one of the nucleotide tags, whereby one label can become linked to one nucleotide tag and a different label can become linked to the other nucleotide tag.
   The amplification mixture is then subjected to amplification, and the fluorescent signal is detected to detect the allele in the sample. In certain embodiments, two differently labeled primers are employed, and the method additionally entails including in the reaction one or more quencher oligonucleotide(s) that include(s) a sequence that is capable of hybridizing to at least part of the nucleotide tag(s) and a fluorescence quencher, wherein hybridization to unincorporated fluorescently labeled primer(s) quenches the fluorescent label(s). In variations of such embodiments, the fluorescence quencher is at the 3′ end of the quencher oligonucleotide or is attached to an internal nucleotide of the quencher oligonucleotide. In specific embodiments, the amplification mixture includes at least two quencher oligonucleotides, one specific for each nucleotide tag.

A fourth method of the invention is a method for adding nucleotide sequences to one or more target nucleic acids by amplification. The method entails preparing an amplification mixture for each target nucleic acid, wherein the amplification mixture includes:
sample nucleic acids;
an inner forward primer including a target-specific sequence and a first nucleotide tag at the 5′ end of the primer;
an inner reverse primer including a target-specific sequence and a second nucleotide tag at the 5′ end of the primer;
an outer forward primer including the first nucleotide tag; and
an outer reverse primer including the second nucleotide tag, wherein one or both outer primers can, optionally, include one or more additional nucleotide sequences to be added to the target nucleic acid.
   Each amplification mixture is subjected to amplification to produce a plurality of target amplicons including tagged target nucleotide sequences, each including first and second nucleotide tags linked to the target nucleotide sequence.

A fifth method of the invention is a method for tagging a plurality of target nucleic acids in a sample with common nucleotide tags. The method entails contacting the sample with:
a plurality of 5′ oligonucleotides, one for each target nucleic acid, wherein each 5′ oligonucleotide includes a first nucleotide tag that is linked, to and 5′ of, a target-specific sequence;
a plurality of 3′ oligonucleotides, one for each target nucleic acid, wherein each 3′ oligonucleotide includes a target-specific sequence that is linked to, and 5′ of, a second nucleotide tag,
wherein the target-specific sequence of each 5′ oligonucleotide hybridizes to a target nucleic acid immediately adjacent to the target-specific sequence of the 3′ oligonucleotide, with an overlap such that one or more of the 5′-most base(s) of the 3′ oligonucleotide is/are displaced from the target nucleic acids, forming a flap;
a flap endonuclease; and
a ligase,
   The contacting is carried under conditions suitable for the flap endonuclease to cleave the flap and the ligase to ligate the 5′ and 3′ oligonucleotides together to produce a plurality of tagged target nucleic acids, each including the first and second tags. After this reaction, the unligated oligonucleotides can be removed and the tagged target nucleic acids amplified using primers specific for the first and second nucleotide tags.

A sixth method of the invention is a method for determining the methylation state of cytosine in a target nucleic acid sequence in a sample. The method entails first treating the sample to convert methylated cytosine(s) to uracil(s) in the target nucleic acids to produce a treated sample, which is contacted with:
a first 5′ oligonucleotide including a first nucleotide tag that is linked to, and 5′ of, a first melting temperature discriminator sequence that is linked to, and 5′ of, a 5′ target-specific sequence, wherein the 3′-most base is a G;
a first 3′ oligonucleotide including a G linked to a 3′ target-specific sequence,
wherein the target-specific sequence of the first 5′ oligonucleotide hybridizes to a target nucleic acid immediately adjacent to the target-specific sequence of the first 3′ oligonucleotide, with an overlap such that at least the G of the 3′ oligonucleotide is displaced from the target nucleic acids, forming a flap;
a second 5′ oligonucleotide including the same first nucleotide tag that is linked to, and 5′ of, a second melting temperature discriminator sequence that is linked to, and 5′ of, a 5′ target-specific sequence, wherein the 3′-most base is an A;
a second 3′ oligonucleotide including an A linked to the 3′ target-specific sequence;
wherein the target-specific sequence of the second 5′ oligonucleotide hybridizes to a target nucleic acid immediately adjacent to the target-specific sequence of the second 3′ oligonucleotide, with an overlap such that at least the A of the 3′ oligonucleotide is displaced from the target nucleic acids, forming a flap;
a flap endonuclease; and
a ligase.
   The contacting is carried under conditions suitable to produce a ligation product from the first 5′ and 3′ oligonucleotides if the target nucleic acid included a methylated cytosine or from the second 5′ and 3′ oligonucleotides if the target nucleic acids included an unmethylated cytosine. After this reaction, the unligated oligonucleotides can, optionally, be removed and the tagged target nucleic acids amplified using a forward primer specific for the first nucleotide tag and a reverse primer that is specific for a target nucleotide sequence in the ligation product. In specific embodiments, melting curve analysis is employed to determine which ligation product was produced.

A seventh method of the invention is method for detecting a relative copy number difference in target nucleic acids in a sample, wherein the method can detect a relative copy number difference less than 1.5. The method entails subjecting a sample to preamplification using primers capable of amplifying a plurality of target nucleic acids to produce a plurality of target amplicons, so that the relative copy numbers of the target nucleic acids is substantially maintained, where some of the target nucleic acids are present on first chromosome and some of the target nucleic acids are present on a second, different chromosome. In various embodiments, at least 10 or at least 100 target on each chromosome of interest are analyzed. After preamplification, the relative copy difference for the first and second chromosomes is determined. In some embodiments, the number of copies of target amplicons derived from the first chromosome and the number of copies of target amplicons derived from the second chromosome are determined by a method that includes amplification. In variations of such embodiments, the amplification comprises digital amplification. In some embodiments, the number of copies of target amplicons derived from the first chromosome and the number of copies of target amplicons derived from the second chromosome are determined by a method that includes DNA sequencing.

An eighth method of the invention is a method for detecting a relative copy number difference between alleles at one or more target loci in a sample including a first allele and a second, different allele at least one target locus, wherein the method can detect a relative copy number difference less than 1.5. The method entails subjecting a sample to preamplification using primers capable of amplifying the first and second alleles to produce a plurality of target amplicons, so that the relative copy numbers of the first and second alleles is substantially maintained. The target amplicons are distributed into a plurality of amplification mixtures, and digital amplification is carried out. The number of amplification mixtures that contain a target amplicon derived from the first allele and the number of amplification mixtures that contain a target amplicon derived from the second allele are determined. The ratio of amplification mixtures that contain the first allele to those that contain the second allele can be determined to detect the relative copy difference for the first and second alleles.

The seventh and eighth methods of the invention can, in certain embodiments, detect relative copy number differences of at least 1.02. In particular embodiments of these methods, preamplification is carried out for between 2 and 25 cycles. In specific embodiments, preamplification is carried out for between 5 and 20 cycles. Both of the methods can include introducing one or more nucleotide tag(s) into the target amplicons. For example, at least one primer of each primer pair employed for preamplification can include a nucleotide tag. Useful nucleotide tags include, e.g., a universal tag and a chromosome-specific nucleotide tag.

A ninth of the invention is a method for detecting fetal aneuploidy in a maternal bodily fluid sample from a pregnant subject, wherein the method can detect a relative chromosomal copy number difference less than 1.5 and, in certain embodiments, at least 1.02. The method entails subjecting a sample of a maternal bodily fluid sample, or a fraction thereof, to preamplification using primer pairs capable of amplifying at least a plurality of target nucleic acids to produce a plurality of target amplicons, so that the relative copy numbers of the target nucleic acids is substantially maintained. Some of the target nucleic acids are present on a first chromosome and some of the target nucleic acids are present on a second, different chromosome. In various embodiments, at least 10 or at least 100 target on each chromosome of interest are analyzed. Each primer employed for preamplification includes a nucleotide tag, so that preamplification produces target amplicons including first a first nucleotide tag at one end and a second nucleotide tag a the other end, wherein all target amplicons derived from a given chromosome include only a few different, or preferably the same, first and second nucleotide tags. All target amplicons derived from a given chromosome are detectable with a common probe. The target amplicons are distributed into a plurality of amplification mixtures, and multiplex digital amplification is carried out using:
a primer pair specific for the first and second nucleotide tags in target amplicons derived from the first chromosome;
a common probe specific for the target amplicons derived from the first chromosome;
a primer pair specific for the first and second nucleotide tags in target amplicons derived from the second chromosome; and
a common probe specific for the target amplicons derived from the second chromosome;
   The number of amplification mixtures that contain a target amplicon derived from the first chromosome and the number of amplification mixtures that contain a target amplicon derived from the second chromosome are determined. From these values the ratio of amplification mixtures that contain the first chromosome to those that contain the second can be determined to detect the relative copy difference for the first and second alleles. In certain embodiments, each common probe detects a chromosome-specific motif. In particular embodiments, motif-specific amplification can be carried out. In illustrative embodiments, the probes are labeled with different fluorescent labels. In particular embodiments of these methods, preamplification is carried out for between 2 and 25 cycles. In specific embodiments, preamplification is carried out for between 5 and 20 cycles.

A tenth method of the invention is a method for detecting a relative copy number difference between at least two loci in genomic DNA or RNA in a sample. The method entails quantifying the amount, in the sample, of a first non-coding RNA expressed from a chromosomal region linked to a first locus, and quantifying the amount, in the sample, of a second non-coding RNA expressed from a chromosomal region linked to a second locus. The ratio of the amount of the first non-coding RNA to the amount of the second non-coding RNA can then be determined, wherein a ratio significantly different from one indicates a copy number difference between the first and second locus. Suitable non-coding RNAs for analysis by this method include single-stranded, non-coding RNAs, double-stranded, non-coding RNAs, and miRNAs.

An eleventh method of the invention is method for detecting a relative copy number difference between at least two loci in genomic DNA a sample. The method entails producing, from the sample, a first DNA sequencing template that includes, 5′ to 3′, a primer binding site for a forward DNA sequencing primer, linked directly, or via an intervening sequence, to a first target nucleotide sequence derived from the first locus, which is linked directly, or via an intervening sequence, to a primer binding site for a reverse DNA sequencing primer. The method further entails producing, from the sample, a second DNA sequencing template that includes, 5′ to 3′, the primer binding site for the forward DNA sequencing primer, linked directly, or via an intervening sequence, to a second target nucleotide sequence derived from the second locus, which is linked directly, or via an intervening sequence, to a primer binding site for the reverse DNA sequencing primer. The forward and reverse DNA sequencing primer binding sites are preferably the same in both DNA sequencing templates, although this is not necessary. The first and second DNA sequencing templates are produced from the sample substantially in proportion to the copy number of the first and second loci in the sample. The nucleotide sequences of the DNA sequencing templates are determined and the amounts of these templates are quantified. A ratio of the amount of the first DNA sequencing template to the amount of the second DNA sequencing template can be determined to determine a copy number difference between the first and second locus. In certain embodiments, the first and second DNA sequencing primers additionally include a barcode nucleotide sequence between the primer binding site for the forward DNA sequencing primer and the first and second target nucleotide sequences, respectively. Alternatively, or in addition, the first and second DNA sequencing primers can additionally include a barcode nucleotide sequence between the first and second target nucleotide sequences, respectively, and the primer binding site for the reverse DNA sequencing primer.

A twelfth method of the invention is method for detecting and/or quantifying one or more fetal target nucleic acids in a maternal bodily fluid sample from a pregnant subject. The method entails treating the sample to enrich for amplifiable fetal nucleic acids and produce a treated sample, wherein the treated sample includes a higher percentage of fetal nucleic acids that are capable of being amplified, as compared to the percentage of maternal nucleic acids that are capable of being amplified. One or more fetal target nucleic acids is/are amplified and detected and/or quantified. In particular embodiments, the maternal bodily fluid is treated to enrich for amplifiable fetal DNA without prior fractionation. Illustrative maternal bodily fluids that can be analyzed in this manner include whole blood, plasma, urine, and cervico-vaginal secretions. In certain embodiments, the treatment includes enriching the sample for short nucleic acids. For example, the treatment can include physical enrichment based on size, e.g., enriching the sample for nucleic acids that are about 300 nucleotides or less in length or about 200 nucleotides or less in length.

In specific embodiments, nucleic acids from a maternal bodily fluid sample are fractionated based on nucleic acid size, and the fractions are assayed to determine which fraction(s) include(s) short nucleic acids. For example, nucleic acid fractions can be queried to determine whether two target nucleic acid sequences that are more than about 300 nucleic acids apart in the genome are found together on individual nucleic acids (characteristic of cell-free maternal DNA) or are found on separate nucleic acids. This determination can be made by hybridization or amplification. In some embodiments, enrichment for short nucleic acids is carried out by selective amplification based on size.

Any of the above-described methods can include forming amplification mixtures, or distributing them into separate compartments of a microfluidic device prior to amplification. In particular embodiments, the microfluidic device can be fabricated, at least in part, from an elastomeric material.

In any of the above-described methods, the sample can be a sample of a maternal bodily fluid, or a fraction thereof, from a pregnant subject. In certain embodiments, of these methods, at least some of the target amplicons, alleles, target nucleic acids, or loci are derived from, or comprise fetal, DNA. In specific embodiments, the sample is a sample of maternal blood, or a fraction thereof, and at least some of the target nucleic acids comprise fetal DNA. These methods can be carried out, for example, to determine a fetal genotype or determine the presence of a mutation or fetal aneuploidy.

### BRIEF DESCRIPTION OF THE DRAWINGS

FIG. 1A-1G: Amplification results from the studies described in Example 1: Use of fluorescent primers and intercalating dye to generate fluorescent PCR signals (real-time, end-point, multiplex). A: LCG, FAM, CalO, CalRed, Quasar; B: Fluorescent primer (CalO) plus EvaGreen; C: Fluorescent primer (CalO) plus EvaGreen (more contrast); D: FAM; E: Fluorescent primer (ROX) plus EvaGreen; F: Fluorescent primer (Quasar) plus EvaGreen; G: Endpoint reads at 20° C., left to right: FM, CalO, CalR, Quasar.

FIG. 2A-2H: Example 2: SNP by tagging and universal fluorescent primers. To detect the allele for a particular locus that is present in a sample, the sample nucleic acids are subjected to allele-specific PCR using two forward allele-specific primers that included 5′ nucleotide tags having different nucleotide sequences and a common reverse primer. A: The amplification reaction includes two tag-specific primers, each with a different fluorescent label at the 5′ end and a double-stranded DNA-binding dye; single-stranded primers give a fluorescent signal; Eva Green binds to the PCR product and quenches signal; B: SNPs 1 to 12: “EP” read after 25 cycles; inverted graphs; C: SNPs 1 to 12: signal is actually negative; D: All calls correct; per SNP, Red and Green indicate the two homozygous GTs; X and Y not matched to allele; XX, XY and YY are the GT calls made; E: PCR protocol used in Example 2; F: Eva Green as reference for SNPs 1-6 gave lines instead of clusters, but can be called; G: Eva Green as reference for SNPs 7-12 gave lines instead of clusters, but can be called; H: Temperature dependence of signal from studies in Example 2.

FIG. 3A-3C: Example 3: Use of target-complementary oligo and a tag-specific primer to generate target-specific tagged primers. A: Complementary oligonucleotide is blocked; only the outer forward primer is extended into the full-length primer; B: Complementary oligonucleotide is not blocked; forward primer and complimentary oligonucleotide are extended into full-length primer and complement; C: Allele-specific long forward primers are generated from extending fluorescent tag primers hybridized to their respective allele's complimentary oligos; in this example, the sample is homozygous for allele A; the fluorescent primers of allele A get incorporated into PCR product and generate fluorescence, while allele B's primers hybridize to a quencher oligonucleotide and generate no fluorescent signal.

FIG. 4A-4C: Example 4: Ligation Assays for Detecting Fetal Aneuploidy. A: FEN cleavage generates a 5′ phosphate on the 3′ oligonucleotide; ligase requires a 5′ phosphate in order to seal DNA nicks; in the absence of FEN cleavage (which requires proper hybridization and alignment), there are no oligos present with a 5′ phosphate and thus no oligos can be ligated together; B: The 5′ oligo and 3′ oligo can be joined using a connector segment so there is only one ligation oligo per assays; upon ligation, a circular ligation product is formed which is resistant to exonuclease digestion; C: Average C_{T} values for each of twelve chrom18 assays performed as described in Example 4.

FIG. 5A-5D: Example 5: Method to Detect Differentially Methylated DNA (i.e., “Methyl SNPs”) Using Tm Enhancing Primers and Fluidgim IFCs. A: Overview of “bisulphite treatment” to discriminate between methylated and unmethylated cytosine (Calladin, Drew et al. 2004); B: Rare SNP- or methylated DNA, ligation, and PCR detection method using Tm enhancing primers (EGFR mutation used as an example for actual results shown in C and D); Results obtained using method shown in B; digital PCR amplicon Tm heat map (C) and Tm melt curves (D) showing specificity of ligation and the change in Tm (° C.) obtained using ligation primers and commercial Fluidigm chips; single SNP Tm differences (3° C.) are readily observed.

FIG. 6A-F: Example 7: Use of pre-amplification and digital PCR for the enhanced detection and quantification of (fetal) aneuploidy, point mutations and SNPs. A: Results of initial study described in Example 7; normalized ratio of chromosome 21 to 18; B: RCN of chromosomes 21 vs. 18 with increasing amount of chromosome 21 spike; the 95% confidence limits for measured values overlap with the expected value (O) in all but one case; using the pooled references 95% CI range (1.00±0.8%) to classify a sample as normal or trisomy, a call of at least >3% difference in chromosome 21 copy number is possible with a 48.770 Digital Array™ IFC; this corresponds to a >6% fetal concentration in maternal plasma; C: RCN of chromosomes 21 vs. 18 measured in pregnancy plasma samples; the RCN was determined for 13 normal pregnancy plasma samples (green), 3 trisomy 21 samples (red) and one trisomy 18 sample (blue); the 99% CI error bars include a sampling error based on input copies of pre-amplification and error of relative quantification of digital PCR; the first, darker bar per sample shows the initial measurement, the second lighter bar the blinded repeat of the same pre-amplification product; D: Effect of long DNA on measured RCN; it was observed a strong correlation between the percentage of long DNA in a sample and the measured RCN; the correlation and trendline are based on the normal pregnancy plasma samples; pregnancy plasma samples with ≧50% long total DNA were excluded; Diamonds: Pregnancy plasma samples, Square: genomic DNA; green: euploid sample, blue: trisomy 18, red: trisomy 21; the average RCN of a sample was plotted where multiple measurements were performed; E: RCN of first and blinded re-test of pre-amplified plasma samples determined by first and second, blind measurement of the same pre-amplification product; Green: euploid pregnancy, Red: trisomy 21 and Blue: trisomy 18; the figure includes results from one trisomy 18 sample that was excluded due to a high proportion of long total DNA; F: the RCN of the first test was set to 1.00; the RCN of the second measurement is in all but one case within ±5% of the RCN of the first measurement of the same pre-amplification product.

FIG. 7A-B: Example 8: A multiplexed approach for detection of fetal aneuploideis in maternal plasma. A: UPL scheme in the quantitation of multiple loci on a single chromosome; colored arrows (red, blue, and green): specific primers for three loci on a chromosome; colored bars (red, blue, and green): three different amplicons; black bars: common tag sequences added to the specific primers and therefore amplicons; the tags added to the forward and reverse primers are different; black arrows: primers used in the digital array quantitation; their sequences are the same as the tags; purple bar: UPL probe that anneals to all 3 amplicons; it is only used in the digital array quantitation; the 3 bars above the chromosome just show its positions in the amplicon; B: Blind test results of 14 pregnancy plasma DNA samples; the green bars represent plasma DNA samples from women pregnant with a normal fetus; the red bars represent samples from trisomy 21-carrying women; the blue bars represent trisomy 18 samples.

FIG. 8A-B: Example 10: Nexgen sequencing detection of fetal aneuploidy with amplicon tagging: A: by pre-PCR; B: by ligation.

FIG. 9: Example 11: SNP detection via target-specific ligation followed by stuffer-based Tm selection: Purpose: Enhanced SNP detection by PreAmp ligation, followed by stuffer-based Tm selection; exemplary target: clinically significant EGFR mutation (Thr ACG-790-to Met-ATG); SNP is engineered with a ΔTm of 13° C. versus 1° C.; generic procedure: (1) Ligation PreAmp with Tm distinguishing stuffers (akin to a standard preamplification); (2) Taq FN activity cleaves flap, revealing a 5′ phosphate group, permitting ligation; cycle 50 times; (3) Asymmetric PCR amplification on a DID-type chip; (4) Compare amplicon Tm difference between Mt (GC-rich stuffer) versus Wt (GC-poor stuffer).

FIG. 10A-M: Example 12: Pre-amplification and amplification methods based on target-specific ligation via LCR/LDR (ligase chain and ligase detection reaction) followed by PCR: A: ligation of multiple (3 or more) neighboring/consecutive probes retains DNA length information, and enriches for these products as only probes hybridizing to the same fragment are ligation competent; a long DNA fragment will yield one long product whereas the same sequence in 10 fragments can yield up to 10 shorter ligation products; performing multiple temperature cycles with a temperature-resistant ligase permits one strand of the target fragment(s) to be linearly amplified up to 500-fold via the ligase-detection/ligase chain assays; B: it is possible to introduce tags for downstream functionalities, such as PCR; tag/tail sequences can be appended at the 5′ end of a ligation probe (left); tags can be added in the middle of a ligation probe (right); in this fashion, both ends of the probes are available for ligation, permitting to produce a ligated chain of probes; C: in one embodiment, the 5′ tag of every 2^{nd} probe can be used as priming site (tag has same sequence as a PCR primer), and the inner tag of every other 2^{nd} probe will serve as binding site of a second primer (and have a linker molecule that halts downstream PCR); this permits selective amplification of the two 5′ most probes, as only the 5′ tag in the ligation product is the one of the first ligation probe (by using 5′ tag and internal tag as primers), regardless of whether the ligation product is long or short; D: in a second embodiment, ligation chain reaction (LCR) using 3 or more consecutive probes per strand (sense and antisense) can serve as a target-specific amplification step that retains target size information; asymmetric LCR/LDR where either the sense or antisense strands are differentially targeted for preferred ligation by use of oligonucleotides that differentially hybridize in a temperature-dependent manner (e.g. enrich for 1^{st} strand product for 100 cycles, prior to switching to a lower ligation temperature prior to LCR or LDR) on the bottom of the figure are two simple embodiments, using chains of probes, etc., may be also positive; E: PCR; F: Ligation: two main schemes of ligation are used in this method as examples: a) 5′-phosphate, b) overhang of one or more nucleotides (Flap) which is cleaved by a flap-endonuclease (e.g. Taq Polymerase) resulting in a ligation competent 5′-phosphate; G: one embodiment of the method entails using more than 2 adjacent probes for ligation; H: in an embodiment, all Forward probes are tagged (e.g. with a common set-specific tag); I: probes can also contain internal tags not complementary to the target sequence; J: another embodiment can entail using a 5′ tag and an internal tag in alternating probes, and PCR of ligation product; K: variations/modifications; L: exo-nuclease resistance; M: further possibilities.

FIG. 11A-B: Example 13: Ligation or PCR-based target-specific Super-Plexing using Universal Sequences and combinatorial tag primers for simultaneous detection of multiple nucleic acid sequences: A: LDR followed by PCR Super-plexing using 2 Universal primers (A and B); employs a combination of only 2 tags to PCR amplify any targeted nucleic acid (RNA shown); general procedure: (1) Hybridize 2 target specific oligos, P1 and P2, each bearing a different tag, to any contiguous nucleic acid; (2) P1 bears a Universal A sequence and Tag 1 sequence at its 5′ end; (3) P2 bears the 5′ overhang FLap-ase target site+a Tag 2 and a Universal B sequence; (4) Taq FEN cleaves the flap, revealing a 5′ phosphate group, permitting ligation; cycle with Ampligase; (5) all ligations will incorporate Universal A and B sequences in the same product; this permits Super-plexing using only 2 Universal primers (A and B); (6) the unique combination of 100 different tag primers o the 5′ primer and 100 different tag primers on the 3′ primer generates 10,000 combinatorial variants representing 10,000 specific primer sets; (7) using 1 tag combination/gene permits exponential amplification of 10,000 separate amplicons; B: LDR followed by PCR Super-plexing using 2 Universal primers (A and B); employs a combination of only 2 tags to PCR amplify nucleic acids; can add a single sense Universal probe library binding site on primer P1 (or 1 of 165 Universal probe library binding sites); general procedure: (1) Hybridize 2 target specific oligos, P1 and P2, each bearing a different tag, to any contiguous nucleic acid; (2) P1 bears a Universal A sequence and Tag 1 sequence at its 5′ end; (3) P1 primer(s) bear(s) a single Universal probe library binding site or 1 of 165 Universal probe library binding sites; probe hydrolysis occurs when P2 primer extends to displace the UPL probe; all amplicons in the single column of a dynamic array contain a single probe sequence in exactly the same sequence context; (4) P2 bears the 5′overhang FLap-ase target site+a Tag 2 and a Universal B sequence; (5) Super-plexing using only 2 Universal primers; (6) the unique combination of 100 different tag primers on the 5′ primer and 100 different tag primers on the 3′ primer permits 10,000 combinations, representing 10,000 specific RNAs or genes to be targeted.

FIG. 12: Example 14: Use of common sequence motifs (with pre-amplification and digital PCR) for the enhanced multiplexing of targets for the detection and quantification of fetal aneuploidy: probes may be employed in the methods described in Example 14 to detect a shared sequence motif; a probe is used that binds (a) to one of the tags of a product and (b) to a common motif for all products that are to be detected by the same probe.

### DETAILED DESCRIPTION

The present invention provides methods for detecting and quantifying target nucleic acids that have general application, but that are particularly well-suited for detecting target nucleic acids of a particular type (e.g., in fetal DNA) that are present in low concentration, together with a much larger amount of non-target nucleic acids (e.g., in maternal DNA).

### Definitions

Terms used in the claims and specification are defined as set forth below unless otherwise specified. These terms are defined specifically for clarity, but all of the definitions are consistent with how a skilled artisan would understand these terms.

The term “adjacent,” when used herein to refer two nucleotide sequences in a nucleic acid, can refer to nucleotide sequences separated by 0 to about 20 nucleotides, more specifically, in a range of about 1 to about 10 nucleotides, or sequences that directly abut one another.

The term “nucleic acid” refers to a nucleotide polymer, and unless otherwise limited, includes known analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; and mRNA.

The term nucleic acid encompasses double- or triple-stranded nucleic acids, as well as single-stranded molecules. In double- or triple-stranded nucleic acids, the nucleic acid strands need not be coextensive (i.e, a double-stranded nucleic acid need not be double-stranded along the entire length of both strands).

The term nucleic acid also encompasses any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like.

More particularly, in certain embodiments, nucleic acids, can include polydeoxyribonucleotides (containing 2-deoxy-D-ribose), polyribonucleotides (containing D- or L-ribose), and any other type of nucleic acid that is an N- or C-glycoside of a purine or pyrimidine base, as well as other polymers containing non-nucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. The term nucleic acid also encompasses linked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

The term “sample nucleic acids” can to refer to nucleic acids (1) in a sample taken directly from a subject, (2) in a fraction of a sample taken directly from a subject, and (3) in a sample, or fraction thereof, that has been subjected to a treatment, such as, e.g., preamplification. Where it is necessary to distinguish among these meanings, clarifying language is used; for example, a “preamplified” sample” or “preamplified” nucleic acids refer to a sample or nucleic acids that have been subjected to preamplification.

The term “target nucleic acids” is used herein to refer to particular nucleic acids to be detected in the methods described herein.

As used herein the term “target nucleotide sequence” refers to a molecule that includes the nucleotide sequence of a target nucleic acid, such as, for example, the amplification product obtained by amplifying a target nucleic acid or the cDNA produced upon reverse transcription of an RNA target nucleic acid.

As used herein, the term “complementary” refers to the capacity for precise pairing between two nucleotides. I.e., if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Specific hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In particular embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference). As indicated by standard references, a simple estimate of the Tₘ value may be calculated by the equation: Tₘ=81.5+0.41(% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7.

Non-coding RNAs include those RNA species that are not necessarily translated into protein. These include, but are not limited to, transfer RNA (tRNA) and ribosomal RNA (rRNA), as well as RNAs such as small nucleolar RNAs (snoRNA; e.g., those associated with methylation or pseudouridylation), microRNAs (miRNA; which regulate gene expression), small interfering RNAs (siRNAs; which are involved in the RNA interference (RNAi) pathway, where they interfere with the expression of specific genes, but have also been shown to act as antiviral agents and in shaping the chromatin structure of a genome) and Piwi-interacting RNAs (piRNAs; which form RNA-protein complexes through interactions with Piwi proteins; these piRNA complexes have been linked to transcriptional gene silencing of retrotransposons and other genetic elements in germ line cells, particularly those in spermatogenesis), and long non-coding RNAs (long ncRNAs; which are non-coding transcripts that are typically longer than about 200 nucleotides).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, more typically range from 10 to 30 nucleotides, or even more typically from 15 to 30 nucleotides, in length. Other primers can be somewhat longer, e.g., 30 to 50 nucleotides long. In this context, “primer length” refers to the portion of an oligonucleotide or nucleic acid that hybridizes to a complementary “target” sequence and primes nucleotide synthesis. Short primer molecules generally require cooler temperatures to form sufficiently stable hybrid complexes with the template. A primer need not reflect the exact sequence of the template but must be sufficiently complementary to hybridize with a template. The term “primer site” or “primer binding site” refers to the segment of the target nucleic acid to which a primer hybridizes.

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in certain embodiments, amplification primers used herein are said to “anneal to a nucleotide tag.” This description encompasses primers that anneal wholly to the nucleotide tag, as well as primers that anneal partially to the nucleotide tag and partially to an adjacent nucleotide sequence, e.g., a target nucleotide sequence. Such hybrid primers can increase the specificity of the amplification reaction.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientation in particular embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe binds or hybridizes to a “probe binding site.” The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15-20 nucleotides in length).

The primer or probe can be perfectly complementary to the target nucleic acid sequence or can be less than perfectly complementary. In certain embodiments, the primer has at least 65% identity to the complement of the target nucleic acid sequence over a sequence of at least 7 nucleotides, more typically over a sequence in the range of 10-30 nucleotides, and often over a sequence of at least 14-25 nucleotides, and more often has at least 75% identity, at least 85% identity, at least 90% identity, or at least 95%, 96%, 97%. 98%, or 99% identity. It will be understood that certain bases (e.g., the 3′ base of a primer) are generally desirably perfectly complementary to corresponding bases of the target nucleic acid sequence. Primer and probes typically anneal to the target sequence under stringent hybridization conditions.

The term “nucleotide tag” is used herein to refer to a predetermined nucleotide sequence that is added to a target nucleotide sequence. The nucleotide tag can encode an item of information about the target nucleotide sequence, such the identity of the target nucleotide sequence or the identity of the sample from which the target nucleotide sequence was derived. In certain embodiments, such information may be encoded in one or more nucleotide tags, e.g., a combination of two nucleotide tags, one on either end of a target nucleotide sequence, can encode the identity of the target nucleotide sequence.

As used herein, the term “encoding reaction” refers to reaction in which at least one nucleotide tag is added to a target nucleotide sequence. Nucleotide tags can be added, for example, by an “encoding PCR” in which the at least one primer comprises a target-specific portion and a nucleotide tag located on the 5′ end of the target-specific portion, and a second primer that comprises only a target-specific portion or a target-specific portion and a nucleotide tag located on the 5′ end of the target-specific portion. For illustrative examples of PCR protocols applicable to encoding PCR, see pending WO Application US03/37808 as well as U.S. Pat. No. 6,605,451. Nucleotide tags can also be added by an “encoding ligation” reaction that can comprise a ligation reaction in which at least one primer comprises a target-specific portion and nucleotide tag located on the 5′ end of the target-specific portion, and a second primer that comprises a target-specific portion only or a target-specific portion and a nucleotide tag located on the 5′ end of the target specific portion. Illustrative encoding ligation reactions are described, for example, in U.S. Patent Publication No. 2005/0260640, which is hereby incorporated by reference in its entirety, and in particular for ligation reactions.

As used herein an “encoding reaction” produces a “tagged target nucleotide sequence,” which includes a nucleotide tag linked to a target nucleotide sequence.

As used herein the term “barcode” refers to a specific nucleotide sequence that encodes information about an amplicon produce during preamplification or amplification. To introduce a barcode into an amplicon, “barcode primer” that includes the barcode nucleotide sequence can be employed in an amplification reaction. For example, a different barcode primer can be employed to amplify one or more target sequences from each of a number of different samples, such that the barcode nucleotide sequence indicates the sample origin of the resulting amplicons.

The term “melting temperature discriminator sequence” refers to a subsequence of a longer double-stranded polynucleotide that renders that polynucleotide distinguishable, by melting temperature, from another polynucleotide, e.g. one containing a different melting temperature discriminator sequence.

As used herein with reference to a portion of a primer, the term “target-specific” nucleotide sequence refers to a sequence that can specifically anneal to a target nucleic acid or a target nucleotide sequence under suitable annealing conditions.

As used herein with reference to a portion of a primer, the term “nucleotide tag-specific nucleotide sequence” refers to a sequence that can specifically anneal to a nucleotide tag under suitable annealing conditions.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. February; 4(1):41-7, U.S. Pat. No. 6,027,998; U.S. Pat. No. 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18—(2002); Lage et al., Genome Res. 2003 February; 13(2):294-307, and Landegren et al., Science 241:1077-80 (1988), Demidov, V., Expert Rev Mol. Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. No. 5,830,711, U.S. Pat. No. 6,027,889, U.S. Pat. No. 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

The term “qPCR” is used herein to refer to quantitative real-time polymerase chain reaction (PCR), which is also known as “real-time PCR” or “kinetic polymerase chain reaction.”

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, and the like. Reagents for enzyme reactions include, for example, substrates, cofactors, buffer, metal ions, inhibitors, and activators.

The term “universal detection probe” is used herein to refer to any probe that identifies the presence of an amplification product, regardless of the identity of the target nucleotide sequence present in the product.

The term “universal qPCR probe” is used herein to refer to any such probe that identifies the presence of an amplification product during qPCR. In particular embodiments, nucleotide tags according to the invention can include a nucleotide sequence to which a detection probe, such as a universal qPCR probe binds. Where a tag is added to both ends of a target nucleotide sequence, each tag can, if desired, include a sequence recognized by a detection probe. The combination of such sequences can encode information about the identity or sample source of the tagged target nucleotide sequence. In other embodiments, one or more amplification primers can include a nucleotide sequence to which a detection probe, such as a universal qPCR probe binds. In this manner, one, two, or more probe binding sites can be added to an amplification product during the amplification step of the methods of the invention. Those of skill in the art recognize that the possibility of introducing multiple probe binding sites during preamplification (if carried out) and amplification facilitates multiplex detection, wherein two or more different amplification products can be detected in a given amplification mixture or aliquot thereof.

The term “universal detection probe” is also intended to encompass primers labeled with a detectable label (e.g., a fluorescent label), as well as non-sequence-specific probes, such as DNA binding dyes, including double-stranded DNA (dsDNA) dyes, such as SYBR Green.

The term “target-specific qPCR probe” is used herein to refer to a qPCR probe that identifies the presence of an amplification product during qPCR, based on hybridization of the qPCR probe to a target nucleotide sequence present in the product.

“Hydrolysis probes” are generally described in U.S. Pat. No. 5,210,015, which is incorporated herein by reference in its entirety for its description of hydrolysis probes. Hydrolysis probes take advantage of the 5′-nuclease activity present in the thermostable Taq polymerase enzyme typically used in the PCR reaction (TagMan® probe technology, Applied Biosystems, Foster City Calif.). The hydrolysis probe is labeled with a fluorescent detector dye such as fluorescein, and an acceptor dye or quencher. In general, the fluorescent dye is covalently attached to the 5′ end of the probe and the quencher is attached to the 3′ end of the probe, and when the probe is intact, the fluorescence of the detector dye is quenched by fluorescence resonance energy transfer (FRET). The probe anneals downstream of one of the primers that defines one end of the target nucleic acid in a PCR reaction. Using the polymerase activity of the Taq enzyme, amplification of the target nucleic acid is directed by one primer that is upstream of the probe and a second primer that is downstream of the probe but anneals to the opposite strand of the target nucleic acid. As the upstream primer is extended, the Taq polymerase reaches the region where the labeled probe is annealed, recognizes the probe-template hybrid as a substrate, and hydrolyzes phosphodiester bonds of the probe. The hydrolysis reaction irrevocably releases the quenching effect of the quencher dye on the reporter dye, thus resulting in increasing detector fluorescence with each successive PCR cycle. In particular, hydrolysis probes suitable for use in the invention can be capable of detecting 8-mer or 9-mer motifs that are common in the human and other genomes and/or transcriptomes and can have a high Tₘ of about 70° C. enabled by the use of linked nucleic acid (LNA) analogs.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation at a wavelength greater than or equal 250 nm. Examples include ethidium bromide, SYBR and EvaGreen DNA binding dyes.

The term “fluorescent dye,” as used herein, generally refers to any dye that emits electromagnetic radiation of longer wavelength by a fluorescent mechanism upon irradiation by a source of electromagnetic radiation, such as a lamp, a photodiode, or a laser.

The term “elastomer” has the general meaning used in the art. Thus, for example, Allcock et al. (Contemporary Polymer Chemistry, 2nd Ed.) describes elastomers in general as polymers existing at a temperature between their glass transition temperature and liquefaction temperature. Elastomeric materials exhibit elastic properties because the polymer chains readily undergo torsional motion to permit uncoiling of the backbone chains in response to a force, with the backbone chains recoiling to assume the prior shape in the absence of the force. In general, elastomers deform when force is applied, but then return to their original shape when the force is removed.

A “polymorphic marker” or “polymorphic site” is a locus at which nucleotide sequence divergence occurs. Illustrative markers have at least two alleles, each occurring at frequency of greater than 1%, and more typically greater than 10% or 20% of a selected population. A polymorphic site may be as small as one base pair. Polymorphic markers include restriction fragment length polymorphism (RFLPs), variable number of tandem repeats (VNTR's), hypervariable regions, minisatellites, dinucleotide repeats, trinucleotide repeats, tetranucleotide repeats, simple sequence repeats, deletions, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site. A transition is the replacement of one purine by another purine or one pyrimidine by another pyrimidine. A transversion is the replacement of a purine by a pyrimidine or vice versa. SNPs can also arise from a deletion of a nucleotide or an insertion of a nucleotide relative to a reference allele.

As used herein, the phrase “the relative copy numbers of the target nucleic acids is substantially maintained” and like phrases indicate that the copy numbers of the target nucleic acids, relative to one another are sufficiently maintained to permit reproducible copy number determinations for the target nucleic acids using the methods described herein.

The term “chromosome-specific motif” is used herein to refer to a nucleotide sequence that is used to identify the presence of a particular chromosome. The motif can, but need not, be absolutely chromosome-specific, such that the motif can be used to unambiguously identify the chromosome, regardless of the presence of other chromosome sequences in an assay mixture. Alternatively, the motif can be one that simply distinguishes one chromosome from another chromosome who sequences of are present in an assay mixture.

Methods of Detecting and/or Quantifying Target Nucleic Acids

A first method of the of the invention is a method for detecting and/or quantifying one or more target amplicon(s) produced by amplification, wherein the detecting and/or quantifying is carried out during amplification or after an amplification endpoint has been reached. The method entails the method including preparing an amplification reaction mixture including:
sample nucleic acids;
at least one target-specific primer pair;
an optional probe, wherein at least one primer of the target-specific primer pair or the probe, if present, is labeled with a fluorescent dye; and
a fluorescent double-stranded DNA-binding dye, where fluorescence from the dye is capable of quenching fluorescent signal from the labeled primer or probe, if present.
   The amplification mixture is then subjected to amplification, and the fluorescent signal is detected to detect and/or quantify the target amplicon(s). This method is based on a signal difference between unicorporated labeled primer or probe and primer or probe that is incorporated into an amplification product. Quenching of the labeled probe or primer can occur via at least two mechanisms: fluorescence resonance energy transfer (FRET) or contact quenching. Depending upon the specific application and reaction conditions, the signal may increase or decrease (quench) as amplification proceeds. In a variation of this first method, at least one of the target-specific primer pair can include a nucleotide tag, and the fluorescent label can be attached to a tag-specific primer.

A second method of the invention is a method for detecting an allele in a sample. The method entails preparing an amplification mixture including:
sample nucleic acids;
two allele-specific primer pairs, wherein:
   at least one primer in each primer pair is specific for an allele and is tagged with a distinct nucleotide tag at the 5′ end of the primer; and
   the other primer in each pair can be the same or different from one another;
at least two differently fluorescently labeled primers or probes, each capable of annealing to one of the nucleotide tags, directly or via one or more intervening primers, whereby one label can become linked to one nucleotide tag and a different label can become linked to the other nucleotide tag.
   The amplification mixture is then subjected to amplification, and the fluorescent signal is detected to detect the allele in the sample.

In certain embodiments, the amplification mixture additionally includes a fluorescent double-stranded DNA-binding dye, wherein (as discussed above) fluorescence from the dye is capable of quenching fluorescent signal from the labeled primers or probes. Depending upon the specific application and reaction conditions, the signal may increase or decrease (quench) as amplification proceeds. In illustrative embodiments, the fluorescence from the dye quenches fluorescent signal from the labeled primers or probes when the labeled primers or probes are incorporated into, or hybridized to, an amplification product. Accordingly, the quenching of the signal corresponding to a particular allele would indicate that this allele was present in the sample.

In particular embodiments, two differently labeled primers are employed, and the method additionally entails including in the reaction one or more quencher oligonucleotide(s) that include(s) a sequence that is capable of hybridizing to at least part of the nucleotide tag(s) and a fluorescence quencher, wherein hybridization to unincorporated fluorescently labeled primer(s) quenches the fluorescent label(s). In variations of such embodiments, the fluorescence quencher is at the 3′ end of the quencher oligonucleotide or is attached to an internal nucleotide of the quencher oligonucleotide. In specific embodiments, the amplification mixture includes at least two quencher oligonucleotides, one specific for each nucleotide tag.

In various embodiments, the quencher oligonucleotide(s) can be greater than 10 nucleotides, greater than 12 nucleotides, greater than 15 nucleotides, greater than 17 nucleotides, or greater than 20 nucleotides in length; about half the length of the fluorescent primer; or greater than half the length of the nucleotide tag. The annealing temperature for the amplification reaction can be, e.g., within about 2, 5, 10, 15, 20, or 25° C. of the melting temperature of the fluorescently labeled primer/quencher hybrid. In various embodiments, the annealing temperature can be at, above, or below the melting temperature of the fluorescently labeled primer/quencher hybrid. Annealing can, for example, be carried out, in a “touchdown” manner, by slowly lowering temperature. In some embodiments, the quencher oligonucleotide is included in the amplification reaction at a lower concentration than the primer so that the reaction may proceed uninhibited. In particular embodiments, the amplification reaction can include one or more additional prime(s), e.g., 5′ (upstream) of the fluorescently labeled primer(s), to drive the efficiency of the amplification reaction. Once enough amplification product has accumulated, it successfully competes with the quencher oligonucleotide for annealing (and extension) of the forward primer.

A third method of the invention is another method for detecting an allele in a sample. The method entails preparing an amplification mixture including:
sample nucleic acids;
two allele-specific oligonucleotides, wherein each oligonucleotide includes a target-specific sequence linked to a distinct 3′ nucleotide tag; and
at least two differently fluorescently labeled primers or probes, each capable of annealing to one of the nucleotide tags, whereby one label can become linked to one nucleotide tag and a different label can become linked to the other nucleotide tag.
   The amplification mixture is then subjected to amplification, and the fluorescent signal is detected to detect the allele in the sample. In certain embodiments, two differently labeled primers are employed, and the method additionally entails including in the reaction one or more quencher oligonucleotide(s) that include(s) a sequence that is capable of hybridizing to at least part of the nucleotide tag(s) and a fluorescence quencher, wherein hybridization to unincorporated fluorescently labeled primer(s) quenches the fluorescent label(s). In variations of such embodiments, the fluorescence quencher is at the 3′ end of the quencher oligonucleotide or is attached to an internal nucleotide of the quencher oligonucleotide. In specific embodiments, the amplification mixture includes at least two quencher oligonucleotides, one specific for each nucleotide tag.

A fourth method of the invention is a method for adding nucleotide sequences to one or more target nucleic acids by amplification. The method entails preparing an amplification mixture for each target nucleic acid, wherein the amplification mixture includes:
sample nucleic acids;
an inner forward primer including a target-specific sequence and a first nucleotide tag at the 5′ end of the primer;
an inner reverse primer including a target-specific sequence and a second nucleotide tag at the 5′ end of the primer;
an outer forward primer including the first nucleotide tag; and
an outer reverse primer including the second nucleotide tag, wherein one or both outer primers can, optionally, include one or more additional nucleotide sequences to be added to the target nucleic acid.
   Each amplification mixture is subjected to amplification to produce a plurality of target amplicons including tagged target nucleotide sequences, each including first and second nucleotide tags linked to the target nucleotide sequence.

A fifth method of the invention is a method for tagging a plurality of target nucleic acids in a sample with common nucleotide tags. The method entails contacting the sample with:
a plurality of 5′ oligonucleotides, one for each target nucleic acid, wherein each 5′ oligonucleotide includes a first nucleotide tag that is linked, to and 5′ of, a target-specific sequence;
a plurality of 3′ oligonucleotides, one for each target nucleic acid, wherein each 3′ oligonucleotide includes a target-specific sequence that is linked to, and 5′ of, a second nucleotide tag,
wherein the target-specific sequence of each 5′ oligonucleotide hybridizes to a target nucleic acid immediately adjacent to the target-specific sequence of the 3′ oligonucleotide, with an overlap such that one or more of the 5′-most base(s) of the 3′ oligonucleotide is/are displaced from the target nucleic acids, forming a flap;
a flap endonuclease; and
a ligase,
   The contacting is carried under conditions suitable for the flap endonuclease to cleave the flap and the ligase to ligate the 5′ and 3′ oligonucleotides together to produce a plurality of tagged target nucleic acids, each including the first and second tags. After this reaction, the unligated oligonucleotides can be removed and the tagged target nucleic acids amplified using primers specific for the first and second nucleotide tags.

A sixth method of the invention is a method for determining the methylation state of cytosine in a target nucleic acid sequence in a sample. The method entails first treating the sample to convert methylated cytosine(s) to uracil(s) in the target nucleic acids to produce a treated sample. The treated sample is then contacted with sodium bisulphite (Frommer, McDonald et al. 1992). New data (Nature, November 2009, (Lister, Pelizzola et al. 2009)) indicates that between 4.3 and 5.8% of cytosine's are methylated. Of these, 99.98% of methylated C occur in the context of the CG dinucleotide. However in human H1 stem cells, 25% of methylation occurs at non-CG sites. Remarkably, this novel 25% of non-CG methylation disappears when embryonic stem cell are induced to differentiate (Lister, Pelizzola et al. 2009). Given this information it is reasonable to assume fetal nucleic acids and nucleosomes bear different epigenetic tags than maternal derived nucleosomes or nucleic acids.

The ability to perform consequent allele- and/or methylation-specific amplification of bisulphite or restriction enzyme treated DNA permits preferential allele specificity. For example, the treated sample can be contacted with:
a first 5′ oligonucleotide including a first nucleotide tag that is linked to, and 5′ of, a first melting temperature discriminator sequence that is linked to, and 5′ of, a 5′ target-specific sequence, wherein the 3′-most base is a G;
a first 3′ oligonucleotide including a G linked to a 3′ target-specific sequence,
wherein the target-specific sequence of the first 5′ oligonucleotide hybridizes to a target nucleic acid immediately adjacent to the target-specific sequence of the first 3′ oligonucleotide, with an overlap such that at least the G of the 3′ oligonucleotide is displaced from the target nucleic acids, forming a flap;
a second 5′ oligonucleotide including the same first nucleotide tag that is linked to, and 5′ of, a second melting temperature discriminator sequence that is linked to, and 5′ of, a 5′ target-specific sequence, wherein the 3′-most base is an A;
a second 3′ oligonucleotide including an A linked to the 3′ target-specific sequence;
wherein the target-specific sequence of the second 5′ oligonucleotide hybridizes to a target nucleic acid immediately adjacent to the target-specific sequence of the second 3′ oligonucleotide, with an overlap such that at least the A of the 3′ oligonucleotide is displaced from the target nucleic acids, forming a flap;
a flap endonuclease; and
a ligase.
   The contacting is carried under conditions suitable for the flap endonuclease to cleave the flap and the ligase to ligate the 5′ and 3′ oligonucleotides together to produce a ligation product from the first 5′ and 3′ oligonucleotides if the target nucleic acid included a methylated cytosine or from the second 5′ and 3′ oligonucleotides if the target nucleic acids included an unmethylated cytosine. After this reaction, the unligated oligonucleotides can be removed and the tagged target nucleic acids amplified using a forward primer specific for the first nucleotide tag and a reverse primer that is specific for a target nucleotide sequence in the ligation product. In specific embodiments, melting curve analysis is employed to determine which ligation product was produced.

A seventh method of the invention is method for detecting a relative copy number difference in target nucleic acids in a sample, wherein the method can detect a relative copy number difference less than 1.5. The method entails subjecting a sample to preamplification using primers capable of amplifying a plurality of target nucleic acids to produce a plurality of target amplicons, so that the relative copy numbers of the target nucleic acids is substantially maintained, where some of the target nucleic acids are present on first chromosome and some of the target nucleic acids are present on a second, different chromosome. In various embodiments, at least 10 or at least 100 target on each chromosome of interest are analyzed. After preamplification, the number of copies of target amplicons derived from the first chromosome and the number of copies of target amplicons derived from the second chromosome are determined by any suitable method, including, e.g., amplification, digital amplification, or DNA sequencing. From these values, the relative copy difference for the first and second chromosomes can be determined.

In certain embodiments, target nucleic acids can be selected based on having a common sequence motif. Primers with the same 3′ end can be employed for amplification. In particular embodiments, target nucleic acids are selected to produce amplicons that contain less than 60% GC, preferably less than 55% GC, or more preferably less than 50% GC. Having an approximately uniform GC-content between different target nucleic acids selects against amplification of long target sequences by lowering the denaturation temperature below 95 C, below 90 C, or below 85 C. In various embodiments, target nucleic acids can be selected that are 100, 200, 500, and/or 1000 basepairs up- and/or downstream of the nucleic acid sequence or region of interest.

In some embodiments, the primers can include nucleotide tags to allow annealing at higher temperature in following cycles, thus avoiding reduced efficiencies due to amplicon secondary structures.

An eighth method of the invention is a method for detecting a relative copy number difference between alleles at one or more target loci in a sample including a first allele and a second, different allele at least one target locus, wherein the method can detect a relative copy number difference less than 1.5. The method entails subjecting a sample to preamplification using primers capable of amplifying the first and second alleles to produce a plurality of target amplicons, so that the relative copy numbers of the first and second alleles is substantially maintained. The target amplicons are distributed into a plurality of amplification mixtures, and digital amplification (described below) is carried out. The number of amplification mixtures that contain a target amplicon derived from the first allele and the number of amplification mixtures that contain a target amplicon derived from the second allele are determined. The ratio of amplification mixtures that contain the first allele to those that contain the second allele can be determined to detect the relative copy difference for the first and second alleles.

The seventh and eighth methods of the invention can, in certain embodiments, detect relative copy number differences of at least 1.02. In particular embodiments of these methods, preamplification is carried out for between 2 and 25 cycles. In specific embodiments, preamplification is carried out for between 5 and 20 cycles. Both of the methods can include introducing one or more nucleotide tag(s) into the target amplicons. For example, at least one primer of each primer pair employed for preamplification can include a nucleotide tag. Useful nucleotide tags include, e.g., a universal tag and a chromosome-specific nucleotide tag.

A ninth of the invention is a method for detecting fetal aneuploidy in a maternal bodily fluid sample from a pregnant subject, wherein the method can detect a relative chromosomal copy number difference less than 1.5 and, in certain embodiments, at least 1.02. The method entails subjecting a sample of a maternal bodily fluid sample, or a fraction thereof, to preamplification using primer pairs capable of amplifying at least a plurality of target nucleic acids to produce a plurality of target amplicons, so that the relative copy numbers of the target nucleic acids is substantially maintained. Some of the target nucleic acids are present on a first chromosome and some of the target nucleic acids are present on a second, different chromosome. In various embodiments, at least 10 or at least 100 target on each chromosome of interest are analyzed. Each primer employed for preamplification includes a nucleotide tag, so that preamplification produces target amplicons including first a first nucleotide tag at one end and a second nucleotide tag a the other end, wherein all target amplicons derived from a given chromosome include only a few different, or preferably the same, first and second nucleotide tags. All target amplicons derived from a given chromosome are detectable with a common probe. The target amplicons are distributed into a plurality of amplification mixtures, and multiplex digital amplification is carried out using:
a primer pair specific for the first and second nucleotide tags in target amplicons derived from the first chromosome;
a common probe specific for the target amplicons derived from the first chromosome;
a primer pair specific for the first and second nucleotide tags in target amplicons derived from the second chromosome; and
a common probe specific for the target amplicons derived from the second chromosome;
   The number of amplification mixtures that contain a target amplicon derived from the first chromosome and the number of amplification mixtures that contain a target amplicon derived from the second chromosome are determined. From these values the ratio of amplification mixtures that contain the first chromosome to those that contain the second can be determined to detect the relative copy difference for the first and second alleles. In certain embodiments, each common probe detects a chromosome-specific motif In particular embodiments, motif-specific amplification can be carried out. In illustrative embodiments, the probes are labeled with different fluorescent labels. In particular embodiments of these methods, preamplification is carried out for between 2 and 25 cycles. In specific embodiments, preamplification is carried out for between 5 and 20 cycles.

In embodiments of this or any of the methods described herein (in particular, those relating to determining copy number differences, target nucleic acids in the Down Syndrome critical region (DSCR) can be analyzed.

A tenth method of the invention is a method for detecting a relative copy number difference between at least two loci in genomic DNA or RNA in a sample. The method entails quantifying the amount, in the sample, of a first non-coding RNA expressed from a chromosomal region linked to a first locus, and quantifying the amount, in the sample, of a second non-coding RNA expressed from a chromosomal region linked to a second locus. The ratio of the amount of the first non-coding RNA to the amount of the second non-coding RNA can then be determined, wherein a ratio significantly different from one indicates a copy number difference between the first and second locus. Suitable non-coding RNAs for analysis by this method include single-stranded, non-coding RNAs, double-stranded, non-coding RNAs, and miRNAs.

An eleventh method of the invention is method for detecting a relative copy number difference between at least two loci in genomic DNA a sample. The method entails producing, from the sample, a first DNA sequencing template that includes, 5′ to 3′, a primer binding site for a forward DNA sequencing primer, linked directly, or via an intervening sequence, to a first target nucleotide sequence derived from the first locus, which is linked directly, or via an intervening sequence, to a primer binding site for a reverse DNA sequencing primer. The method further entails producing, from the sample, a second DNA sequencing template that includes, 5′ to 3′, the primer binding site for the forward DNA sequencing primer, linked directly, or via an intervening sequence, to a second target nucleotide sequence derived from the second locus, which is linked directly, or via an intervening sequence, to a primer binding site for the reverse DNA sequencing primer. The forward and reverse DNA sequencing primer binding sites are preferably the same in both DNA sequencing templates, although this is not necessary. The first and second DNA sequencing templates are produced from the sample substantially in proportion to the copy number of the first and second loci in the sample. The nucleotide sequences of the DNA sequencing templates are determined and the amounts of these templates are quantified. A ratio of the amount of the first DNA sequencing template to the amount of the second DNA sequencing template can be determined to determine a copy number difference between the first and second locus. In certain embodiments, the first and second DNA sequencing primers additionally include a barcode nucleotide sequence between the primer binding site for the forward DNA sequencing primer and the first and second target nucleotide sequences, respectively. Alternatively, or in addition, the first and second DNA sequencing primers can additionally include a barcode nucleotide sequence between the first and second target nucleotide sequences, respectively, and the primer binding site for the reverse DNA sequencing primer.

A twelfth method of the invention is method for detecting and/or quantifying one or more fetal target nucleic acids in a maternal bodily fluid sample from a pregnant subject. The method entails treating the sample to enrich for amplifiable fetal nucleic acids and produce a treated sample, wherein the treated sample includes a higher percentage of fetal nucleic acids that are capable of being amplified, as compared to the percentage of maternal nucleic acids that are capable of being amplified. One or more fetal target nucleic acids is/are amplified and detected and/or quantified. In particular embodiments, the maternal bodily fluid is treated to enrich for amplifiable fetal DNA without prior fractionation. Illustrative maternal bodily fluids that can be analyzed in this manner include whole blood, plasma, urine, and cervico-vaginal secretions. In certain embodiments, the treatment includes enriching the sample for short nucleic acids. For example, the treatment can include physical enrichment based on size, e.g., enriching the sample for nucleic acids that are about 300 nucleotides or less in length or about 200 nucleotides or less in length.

In some embodiments, the method entails using whole blood (or other un-frationated bodily fluid and generating a sequencing library (e.g. as described with plasma by Quake and Lo independently in PNAS ˜2008 by blunt-ending DNA fragment and blunt-end ligation of sequencing adapters), while at the same time enriching for short fragments. If proceeding to sequencing, the sequencing method may further bias in favor of shorter (including fetal) fragments and/or the sequencing library can be size-separated.

In specific embodiments, nucleic acids from a maternal bodily fluid sample are fractionated based on nucleic acid size, and the fractions are assayed to determine which fraction(s) include(s) short nucleic acids. For example, nucleic acid fractions can be queried to determine whether two target nucleic acid sequences that are more than about 300 nucleic acids apart in the genome are found together on individual nucleic acids (characteristic of cell-free maternal DNA) or are found on separate nucleic acids (characteristic of cell-free fetal DNA). This determination can be made by hybridization or amplification.

Alternatively a selective protection and/or tagging method can be carried to enrich for amplifiable fetal nucleic acids, as described below in the section entitled Enhancing Target Sequence Populations in a Sample of Mixed Length Nucleic Acids.”

The twelfth method can be carried out, e.g., to determine a fetal genotype or determine the presence of a mutation or fetal aneuploidy.

Other methods that can be combined with those described herein are found in commonly owned, co-pending application Ser. No. 12/548,132 (filed Aug. 26, 2009; Attorney Docket No. FLUDP002), Ser. No. 12/687,018 (filed Jan. 13, 2010; Attorney Docket No. FLUDP005), Ser. No. 12/695,010 (filed Jan. 27, 2010; Attorney Docket No. FLUDP006), Ser. No. 12/753,703 (filed Apr. 2, 2010; Attorney Docket No. FLUDP007), and Ser. No. 12/752,974 (filed Apr. 1, 2010; Attorney Docket No. FLUDP008).

General Approaches for Increasing the Accuracy and/or Precision of Relative Copy Number Determination by Amplification

The detection of fetal aneuploidy in a maternal bodily fluid sample (e.g., plasma) requires a significantly higher assay accuracy and precision than has been achieved previously. The methods described herein facilitate the detection of copy number differences of less than 1.5-fold. In various embodiments, the methods permit detection of copy number differences of 1.45-fold, 1.4-fold, 1.35-fold, 1.3-fold, 1.25-fold, 1.2-fold, 1.15-fold, 1.1-fold, 1.09-fold, 1.08-fold, 1.07-fold, 1.06-fold, 1.05-fold, 1.04-fold, 1.03-fold, or 1.02-fold or less, or a copy number difference falling within any range bounded by any two of the above values. The required precision is readily achieved using one or more of the several approaches described herein, individually or in combination.

First, one can preamplify the target nucleic acid sequence before analysis by amplification. Preamplification increases the number of target and/or internal control nucleic acids, which renders subsequent relative copy number determinations more accurate and precise. In particular embodiments, the target sequence and an internal control sequence are preamplified in parallel, typically, at the same time, under the same reaction conditions, and, more typically, in the same reaction mixture. Generally, the preamplification is carried out for a relatively small number of cycles, so that the relative amounts of the target and internal control sequences is substantially unaltered by the preamplification step. More specifically, the preamplification should be sufficiently proportionate that copy number differences of less than 1.5-fold can be detected in the subsequent amplification reaction. In various embodiments, preamplification is carried out for between 5 and 25 cycles, e.g., for 5, 6, 7, 8, 9, 10, 11, 12, 13, 14, 15, 16, 17, 18, 19, 20, 21, 22, 23, 24, or 25 cycles. In illustrative embodiments, preamplification is carried out for between 10 and 20 cycles.

A second approach to increase the accuracy and/or precision of the relative copy number determination is to carry out a large number of parallel preamplification and/or amplification reactions (i.e., replicates). The use of replicates in preamplification can increase the accuracy of the subsequent relative copy number determination, and the use or replicates during amplification/quantification can increase the precision of this determination. In specific embodiments, each preamplification and/or amplification reaction (i.e., for each sample and/or each nucleic acid sequence of interest) is carried out in at least 4, 6, 8, 10, 12, 16, 24, 32, 48, 50, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, 9500, or 10,000 or more replicates. Furthermore, the number of replicates can be within any range having any of these values as endpoints.

In illustrative embodiments, a sample is divided into aliquots and preamplified, and then each preamplified aliquot is divided into further aliquots and subjected to amplification.

An approach to increasing the accuracy and precision of aneuploidy determinations is to analyze a plurality of target sequences on the chromosome of interest. In illustrative embodiments, 2, 3, 4, 5, 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, 1000 or more target and/or internal control sequences on a chromosome of interest are analyzed. In addition, any number of sequences falling within ranges bounded by any of these values can be analyzed.

Considerations for Preamplification/Amplification

In certain embodiments, the length of the target and/or internal control sequences is relatively short, e.g., such that preamplification and/or amplification produces amplicons including fewer than 200, 175, 150, 125, 100, 75, 50, 45, 40, 35, or 30 nucleotides or amplicons having a length within any range bounded by these values. In specific embodiments, primer pairs wherein the primers bind to overlapping target sequences can be employed. The overlap can be, e.g., 1, 2, or 3 nucleotides. Assay methods employing small amplicons are useful for applications aimed at determining copy number in samples containing fragmented nucleic acids, as is the case, e.g., for cell-free fetal DNA in a maternal bodily fluid (e.g., plasma), cell-free DNA in the bodily fluid (e.g., plasma) of subjects with cancer, or DNA from formalin-fixed paraffin-embedded tissue.

Relatively long annealing times and/or lower than usual annealing temperatures can be employed in particular embodiments, e.g., where the target and/or internal control sequences are present at a relatively low concentration in the sample (e.g., as in the case of cell-free fetal DNA in maternal plasma). In illustrative embodiments, these conditions can be employed, individually or together, during preamplification. Illustrative longer-than-usual annealing times include more than 30 seconds, and more than 60 seconds, more than 120 seconds, more than 240 seconds, more than 10 minutes, more than 1 hour, or more than 10 hours, or any time falling within a range bounded by any of these values. Longer annealing times are typically employed in highly multiplexed reactions and/or reactions where primer concentrations are relatively low. Illustrative lower-than-usual annealing temperatures include less than 65° C., less than 60° C., less than 55° C., less than 50° C., and less than any temperature falling within a range bounded by any of these values.

In particular embodiments, the preamplification step can be used to introduce a nucleotide tag. For example, at least one primer of each primer pair employed for preamplification can include a nucleotide tag, which becomes incorporated into the preamplified nucleic acids. The nucleotide tag can include any desired sequence, e.g., one that encodes an item of information about the target and/or internal control sequence and/or one that includes a primer binding site and/or a probe binding site. In illustrative embodiments, the nucleotide tag includes a universal tag and/or a common tag. A common tag can be introduced into a plurality of target and/internal control sequences. For example, a common chromosome-specific tag can be introduced into all sequences preamplified from a particular chromosome.

To introduce one or more nucleotide tags during preamplification, one or more primers include a target-specific portion and a nucleotide tag. In the first cycle of amplification, only the target-specific portion anneals to the target nucleic acid sequence (or internal control sequence). If both primers in each primer pair are tagged, the same is true for the second cycle of amplification. During these cycles, the annealing temperature should be suitable for annealing of the target-specific portion(s) of the primer(s). Subsequently, however, the annealing temperature can be increased to increase the stringency of the annealing, and thereby favor the amplification of tagged target and/or tagged internal control sequences.

If one or more tags is/are introduced into each target and/or internal control sequence, amplification/quantification can be carried out using one or more tag-specific primers. So, for example, if common nucleotide tags are employed, common tag-specific primers can be used to produce amplicons for detection. Such primers could introduce a binding site for a universal detection probe such that detection could be carried out using a single probe for multiple sequences.

Enhancing Target Sequence Populations in a Sample of Mixed Length Nucleic Acids

Methods are provided for enhancing a nucleic acid sample for target sequences of interest and/or selectively tagging those sequences. These enrichment/selective tagging methods can be combined with methods described above to further facilitate the detection and or quantification of target sequences in samples having mixed length nucleic acids (e.g. fetal DNA in maternal plasma or tumor DNA in plasma from cancer patients.

In certain embodiments, methods are provided for protecting target sequences from exonuclease digestion thereby facilitating the elimination in a sample of undesired amplification primers and/or a portion of certain background sequences (e.g., maternal DNA).

Methods are also provided for selectively tagging short (e.g., fetal DNA) sequences in a sample comprising long and short nucleic acids by using inner tagged forward and reverse primers (one or both tagged) in combination with outer primers in a nucleic acid amplification (e.g., PCR) mix. As explained below, shorter (e.g., fetal) target nucleic acids are amplified and tagged while the amplification of longer (e.g., maternal nucleic acid sequences) is suppressed by one or more mechanisms including blocking of extension of the inner primers by prior annealing and extension of the outer primers, TaqMan 5′ endonuclease digestion of the inner primer and/or its extension product by extension of the outer primer, and/or displacement of the inner tagged product and exonuclease digestion after amplification cycle 1 or 2.

Some embodiments, entail the use of one or more outer primers (or capture probe, i.e. no amplification need be carried out) linked to a moiety that can be used to remove these sequences (e.g., biotin). Alternatively, one or more inner primer may be linked to such a moiety. If such (an inner or outer) primer is extended prior to separation, it may or may not be separated from target sequence. Extension can be carried out to provide a stronger binding to the target sequence.

Selective Protection of Target Sequences from Enzymatic Degradation

In certain embodiments methods are provided for the selective protection of target nucleic acid sequences from enzymatic degradation. Accordingly, in certain embodiments, the methods comprise denaturing sample nucleic acids in a reaction mixture; contacting the denatured sample nucleic acids with at least one target-specific primer pair under suitable annealing conditions; conducting a first cycle of extension of any annealed target-specific primer pairs by nucleotide polymerization; and after the first cycle of extension, conducting a first cycle of nuclease digestion of single-stranded nucleic acid sequences in the reaction mixture. In various embodiments the methods can further involve denaturing the nucleic acids in the reaction mixture after the first cycle of nuclease digestion; contacting the denatured nucleic acids with at least one target-specific primer pair under suitable annealing conditions; conducting a second cycle of extension of any annealed target-specific primer pairs by nucleotide polymerization; and conducting a second cycle of nuclease digestion of single-stranded nucleic acid sequences in the reaction mixture. The process can optionally be repeated for additional cycles as required. In certain embodiments the same target-specific primer pair is used to prime each of the first and second cycles of extension, while in other embodiments, different target-specific primer pairs are used for the first and second cycle. Any of a variety of nucleases that preferably digest single stranded nucleic acids can be used. Suitable nucleases include for example a single strand-specific 3′ exonuclease, a single strand-specific endonuclease, a single strand-specific 5′ exonuclease, and the like. In certain embodiments the nuclease comprises E. coli Exonuclease I. In certain embodiments the nuclease comprises a reagent such as ExoSAP-ITC). ExoSAP-IT® utilizes two hydrolytic enzymes, Exonuclease I and Shrimp Alkaline Phosphatase, together in a specially formulated buffer to remove unwanted dNTPs and primers from PCR products. Exonuclease I removes residual single-stranded primers and any extraneous single-stranded DNA produced in the PCR. Shrimp Alkaline Phosphatase removes the remaining dNTPs from the PCR mixture. In certain embodiments ExoSAP-IT is added directly to the PCR product and incubated at 37° C. for 15 minutes. After PCR treatment, ExoSAP-IT® is inactivated simply by heating, e.g., to 80° C. for 15 minutes.

In certain embodiments the target-specific primers comprise dU, rather than dT, and dUTP, rather than dTTP, is present in the reaction mixture. In certain embodiments the methods additionally comprise contacting the reaction mixture with E. coli Uracil-N-Glycosylase after the second cycle of nuclease digestion. In one illustrative embodiment, the method is carried out using two or more target-specific primer pairs, where each primer pair is specific for a different target nucleotide sequence. In various embodiments, particular, where the target specific primers introduced nucleotide tags, the method can involve after the second cycle of nuclease digestion, denaturing the nucleic acids in the reaction mixture; contacting the denatured nucleic acids with at least one target (e.g., tag) specific primer pair under suitable annealing conditions; and amplifying the corresponding (e.g., tagged) target nucleotide sequence.

In certain embodiments, “primers” (or probes) that hybridize to target need not be extended. If, for example, 3′-exonuclease is employed, the primer will block digestion of the target strand at a certain position, which will become the 3′ end of the remaining target strand, while all sequences upstream of the target will be protected, whether double stranded (paired with primer/probe) or single stranded.

Selective Tagging of Short Target Sequences

In certain embodiments methods are provided for selectively tagging short target sequences (e.g., cell free fetal DNA) in a mixed population of short and long nucleic acids (e.g., cell free DNA obtained from maternal plasma). In various embodiments the method typically involves performing a nucleic acid amplification using a set of nested primers comprising inner primers and outer primers. In various embodiments one or both of the inner can be tagged to thereby introduce a tag onto the target amplification product.

The outer primers do not anneal on the short fragments (e.g., fetal DNA) that carry the (inner) target sequence. The inner primers (labeled “I” in the figure) anneal to the short fragments and generate an amplification product that carries a tag and the target sequence. After 2 cycles a short double stranded fragment generates two double stranded products (which are 3′-exonuclease resistant). One strand of each of these carries both tags (where both primers were tagged).

At the same time, tagging of the long fragments (e.g., maternal DNA) is inhibited. This occurs through a combination of mechanisms. First, the extension of the inner primers can be blocked by the prior annealing and extension of the outer primer. Second, the extension of the outer primer can lead to cleavage of the tag from the already annealed inner primer. The third possibility is that the inner primers' extension product is displaced but intact. The result is that after two cycles, target sequences on the short nucleic acids (e.g., cell free fetal DNA) are tagged, while the longer nucleic acids (e.g., cell free maternal DNA), even those containing the target nucleotide sequence, are not tagged. Moreover, the tagged amplification products from the short sequences are double stranded and thereby 3′-exonuclease resistant.

At this point, enrichment for tagged target sequences (e.g., fetal DNA) can readily be accomplished by any of a variety of methods. For example, an exonuclease digestion can be performed (e.g., as described above) to digest all non-double stranded sequences including extension products of displaces inner primers. This removes the majority of genomic DNA background, while the target sequence are double stranded and stay intact. This also removes substantially all leftover primers.

In certain embodiments after the first cycle, and preferably after second cycle it is possible to directly continue thermocycling (e.g., without exonuclease digestion), but increasing the annealing temperature (e.g., from 60° C. to 72° C.). As a consequence, the inner primers will amplify only sequences that are tagged. The primers cannot bind to untagged target sequences.

In certain embodiments the denaturation temperature is selected to avoid melting of the long DNA amplification product(s). This can be applied right at the first cycle or after a limited amount of amplification rounds, when the short fragments have formed a PCR product that will melt at low temperatures (e.g., 70° C.-80° C.).

In certain embodiments the primers used for further amplification (e.g., after the first cycle and preferably after the second cycle) are specific to the two tags and not to the target sequences.

The resulting amplified tagged target sequences can be analyzed by any convenient methods. Such methods include, for example several modes of PCR (or other amplification methods). Several choices of how to encode target sequences by tagging can be selected. Straightforward is digital PCR. To multiplex several targets (e.g. per chromosome 21), these targets can be encoded with the same two tags. For each chromosome one could use only one primer pair in the PCR reaction.

Accordingly, in certain embodiments, methods are provided for selective tagging of short nucleic acids comprising a short target nucleotide sequence (nucleic acid) over longer nucleic acids comprising the same target nucleotide sequence. In various embodiments the method involves denaturing sample nucleic acids in a reaction mixture, where the sample nucleic acids comprise long nucleic acids and short nucleic acids, each comprising the same target nucleotide sequence. The denatured sample nucleic acids are contacted with one or preferably at least two target-specific primer pairs under suitable annealing conditions, where the primer pairs comprise an inner primer pair (one or both carrying a nucleotide tag, e.g., a 5′ nucleotide tag) that can amplify the target nucleotide sequence on long and short nucleic acids; and an outer primer pair that amplifies the target nucleotide sequence on long nucleic acids, but not on short nucleic acids. A first cycle of extension is conducted for any annealed primer pairs by nucleotide polymerization. After the first cycle of extension, the nucleic acids in the reaction mixture are denatured, the reaction mixture is subjected to suitable annealing conditions; and a second cycle of extension is conducted to produce at least one tagged target nucleotide sequence that comprises two nucleotide tags, one from each inner primer, with the target nucleotide sequence located between the nucleotide tags. It will be recognized that in certain embodiments, one use primers for only one strand in a simple mode, or for one strand per cycle.)

In certain embodiments, the method can additionally involve digesting single-stranded nucleic acid sequences in the reaction mixture after the first and/or the second cycle. In certain embodiments the digestion can by the use of an endonuclease (e.g., single strand-specific 3′ exonuclease, single strand-specific endonuclease, a single strand-specific 5′ exonuclease, a combination of exonuclease alkaline phosphatase, etc.), e.g., as described above. The nuclease treatment digests substantially all non-double stranded sequences (including remaining primers, extension products of displaced inner primers, etc.), removes a substantial portion of gDNA background while leaving intact the double stranded target sequences.

In certain embodiments, as a substitute for the digestion, or in addition to the digestion, the method additionally comprises adding additional quantities the same or different target-specific primer pairs to the reaction mixture and performing one or more amplification cycles to preferentially amplify the tagged target sequences.

In certain embodiments after the first cycle of extension, any subsequent denaturation is carried out at a sufficiently low temperature (e.g. about 80° C. to about 85° C.) to avoid denaturation of any extension product of the outer primer pair.

In certain preferred embodiments, the method additionally comprises subjecting the reaction mixture to one or more cycles of amplification, wherein annealing is carried out at a sufficiently high temperature that the inner primers will only anneal to tagged target nucleotide sequences. This can be during the first to cycles and/or after the first two amplification cycles.

In certain embodiments the method(s) additionally involve contacting the at least one tagged target nucleotide sequence with a tag-specific primer pair under suitable annealing conditions; and amplifying the tagged target nucleotide sequence or using other modes of detection and/or quantification, e.g. as described herein. In certain embodiments the method further involves detecting and/or quantifying the amount of at least one tagged target nucleotide sequence produced by amplification (e.g., via digital PCR (dPCR)).

In certain embodiments the “short” nucleic acid fragments are less than about 500 nucleotides, preferably less than about 400, more preferably less than about 350 nucleotides, and most preferably about 300 nucleotides or shorter (e.g., 250 nt, 200 nt, etc.).

While the methods described herein can be used with essentially any nucleic acid sample comprising long and short nucleic acids (nucleic acid molecules), in certain embodiments, the short nucleic acids comprise fetal nucleic acids (e.g., cell free fetal DNA from maternal plasma or urine), while the long nucleic acids comprise maternal nucleic acids (e.g., cell free maternal DNA from plasma or urine). In various embodiments the nucleic acid are derived from a maternal biological sample (e.g., a biological sample from a pregnant mammal (e.g., human) comprising maternal plasma, maternal urine, amniotic fluid, etc.). In certain embodiments the nucleic acids are derived from a biological sample from a mammal (e.g., a human or non-human mammal) having, suspected of having, or at risk for, a pathology or congenital disorder characterized by a nucleic acid abnormality (e.g., aneuploidy, fragmentation, amplification, deletion, single-nucleotide polymorphism, translocation, chromosomal rearrangement or resorting, etc.). In certain embodiments the nucleic acids are derived from a biological sample from a mammal (e.g., a human or non-human mammal) having, suspected of having, or at risk for a cancer. In certain embodiments, the short nucleic acid fragments comprise tumor or metastatic cell DNA, and the long nucleic acids comprise normal DNA.

In certain embodiments the method can be used to determine linkage of two sequence that are relatively neighboring. For example, if an upstream SNP has, for example a “G” nucleotide and the suppression primer(s) are designed to bind to this sequence then amplification of this SNP is suppressed. If the base is an A, the primers bind inefficiently and don't suppress indicating the presence of the A form sequence.

In various embodiments the inner and outer primers are designed/selected so the distance from outer primers to the target nucleotide sequence (measured as the number of nucleotides between the 5′ ends and thereby including the length of both primers) ranges from about 50, 80, 100, 120, 130, 140, or 150 nucleotides or greater. In certain embodiments, the distance from outer primers to the target nucleotide ranges from about 50, 80, 100, 120, 130, 140, or 150 nucleotides to about 400, 350, 300, 250, or 200 nuclides. For selectively tagging fetal versus maternal cell free nucleic acids, the distance from each outer primer to the target nucleotide sequence is greater than about 130 nucleotides, and typically ranges from about 150 to about 200 nucleotides.

It will be recognized that, in certain embodiments, a large number of different target sequences (e.g., 2 or more, 3 or more, 5 or more, 10 or more, 15 or more, 20 or more, 50 or more, 100 or more per chromosome or other template(s)), can be tagged. Moreover using various tagging strategies, different amplification produces are readily discriminated thereby permitting the methods to be highly multiplexed.

In certain embodiments, fetal aneuploidy via Cts can be determined using for example tag-specific primers for pre-amplification (e.g. one primer pair for preamp after 2 tagging cycles), and then again using target specific primers for real-time PCR, e.g., in a chip.

In certain embodiments it is contemplated to apply digital PCR (dPCR) or amplification and dPCR or fetal aneuploidy via CTS to the tagged short fragments. In certain illustrative embodiment the methods are not only useful for determining/detecting fetal aneuploidy but also for fetal genotyping (SNPs), mutation detection (including sequencing), methylation analysis, and the like.

In certain embodiments, inner primer can also be modified in another way, such that after 1, 2, 3, or more amplification cycles, products can be selectively removed from long targets. For example, an inner primer can be 5′-protected and long products digested by exonucleases. Alternatively, an inner prime can be modified (e.g., biotinylated) for capture.

Outer primers can be tagged such that they will not further amplify under the reaction conditions. For example, outer primers can be tagged with GC rich tags, so that the melting temperature (Tm) is above the T(denaturation) employed. Alternatively, outer primes can be designed such that the reverse complement product loops back onto itself, thereby being further extended by polymerase and forming a long stem that is not denatured or that closes again, thereby preventing annealing of inner primer and further amplification.

It is also possible to selectively tag the long sequences to remove them, including after a number of amplification cycles.

### Sample Nucleic Acids

Preparations of nucleic acids (“samples”) can be obtained from biological sources and prepared using conventional methods known in the art. In particular, DNA or RNA useful in the methods described herein can be extracted and/or amplified from any source, including bacteria, protozoa, fungi, viruses, organelles, as well higher organisms such as plants or animals, particularly mammals, and more particularly humans. Suitable nucleic acids can also be obtained from environmental sources (e.g., pond water), from man-made products (e.g., food), from forensic samples, and the like. Nucleic acids can be extracted or amplified from cells, bodily fluids (e.g., blood, a blood fraction, urine, etc.), or tissue samples by any of a variety of standard techniques. Illustrative samples include samples of plasma, serum, spinal fluid, lymph fluid, peritoneal fluid, pleural fluid, oral fluid, and external sections of the skin; samples from the respiratory, intestinal genital, and urinary tracts; samples of tears, saliva, blood cells, stem cells, or tumors. For example, samples of fetal DNA can be obtained from an embryo or from maternal blood. Samples can be obtained from live or dead organisms or from in vitro cultures. Illustrative samples can include single cells, paraffin-embedded tissue samples, and needle biopsies. Nucleic acids useful in the invention can also be derived from one or more nucleic acid libraries, including cDNA, cosmid, YAC, BAC, P1, PAC libraries, and the like.

In specific embodiments, the sample includes a sample of a maternal bodily fluid, or a fraction thereof, from a pregnant subject. For example, samples of whole blood, plasma, urine, and/or cervico-vaginal secretions can be employed in the methods described herein

Nucleic acids of interest can be isolated using methods well known in the art, with the choice of a specific method depending on the source, the nature of nucleic acid, and similar factors. The sample nucleic acids need not be in pure form, but are typically sufficiently pure to allow the amplification steps of the methods of the invention to be performed. Where the target nucleic acids are RNA, the RNA can be reversed transcribed into cDNA by standard methods known in the art and as described in Sambrook, J., Fritsch, E. F., and Maniatis, T., Molecular Cloning: A Laboratory Manual. Cold Spring Harbor Laboratory Press, NY, Vol. 1, 2, 3 (1989), for example. The cDNA can then be analyzed according to the methods of the invention.

### Target Nucleic Acids

Any target nucleic acid that can be tagged in an encoding reaction of the invention (described herein) can be detected using the methods of the invention. In typical embodiments, at least some nucleotide sequence information will be known for the target nucleic acids. For example, if the encoding reaction employed is PCR, sufficient sequence information is generally available for each end of a given target nucleic acid to permit design of suitable amplification primers. In an alternative embodiment, the target-specific sequences in primers could be replaced by random or degenerate nucleotide sequences.

The targets can include, for example, nucleic acids associated with pathogens, such as viruses, bacteria, protozoa, or fungi; RNAs, e.g., those for which over- or under-expression is indicative of disease, those that are expressed in a tissue- or developmental-specific manner; or those that are induced by particular stimuli; genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations).

In specific embodiments, at least some of the target amplicons, alleles, target nucleic acids, or loci analyzed according to the methods herein are derived from, or include fetal, DNA. For example, the sample to be analyzed can include a sample of a maternal bodily fluid, such as blood, or a fraction thereof, and at least some of the target nucleic acids can include fetal DNA.

### Primer Design

Primers suitable for nucleic acid amplification are sufficiently long to prime the synthesis of extension products in the presence of the agent for polymerization. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 15 to about 30 nucleotides, although it may contain more or fewer nucleotides. The primers should be sufficiently complementary to selectively anneal to their respective strands and form stable duplexes. One skilled in the art knows how to select appropriate primer pairs to amplify the target nucleic acid of interest.

For example, PCR primers can be designed by using any commercially available software or open source software, such as Primer3 (see, e.g., Rozen and Skaletsky (2000) Meth. Mol. Biol., 132: 365-386; www.broad.mit.edu/node/1060, and the like) or by accessing the Roche UPL website. The amplicon sequences are input into the Primer3 program with the UPL probe sequences in brackets to ensure that the Primer3 program will design primers on either side of the bracketed probe sequence.

In certain embodiments, primers including nucleotide tags can be designed so that they form a stem-loop structure to avoid increased mis-hybridization because of nucleotide tag. In some embodiments, a nucleotide tag can be blocked by a complementary oligonucleotide that binds to it during the annealing step to prevent the nucleotide tag from contributing to non-specific hybridization and mis-priming.

Primers may be prepared by any suitable method, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; the solid support method of U.S. Pat. No. 4,458,066 and the like, or can be provided from a commercial source.

Primers may be purified by using a Sephadex column (Amersham Biosciences, Inc., Piscataway, N.J.) or other methods known to those skilled in the art. Primer purification may improve the sensitivity of the methods of the invention.

### Quantitative Real-Time PCR and Other Detection and Quantification Methods

Any method of detection and/or quantification of nucleic acids can be used in the invention to detect amplification products. In one embodiment, PCR (polymerase chain reaction) is used to amplify and/or quantify target nucleic acids. In other embodiments, other amplification systems or detection systems are used, including, e.g., systems described in U.S. Pat. No. 7,118,910 (which is incorporated herein by reference in its entirety for its description of amplification/detection systems) and Invader assays; PE BioSystems). In particular embodiments, real-time quantification methods are used. For example, “quantitative real-time PCR” methods can be used to determine the quantity of a target nucleic acid present in a sample by measuring the amount of amplification product formed during the amplification process itself.

Fluorogenic nuclease assays are one specific example of a real-time quantification method that can be used successfully in the methods described herein. This method of monitoring the formation of amplification product involves the continuous measurement of PCR product accumulation using a dual-labeled fluorogenic oligonucleotide probe—an approach frequently referred to in the literature as the “TaqMan® method.” See U.S. Pat. No. 5,723,591; Heid et al., 1996, Real-time quantitative PCR Genome Res. 6:986-94, each incorporated herein by reference in their entireties for their descriptions of fluorogenic nuclease assays. It will be appreciated that while “TaqMan® probes” are the most widely used for qPCR, the invention is not limited to use of these probes; any suitable probe can be used.

Other detection/quantification methods that can be employed in the present invention include FRET and template extension reactions, molecular beacon detection, Scorpion detection, Invader detection, and padlock probe detection.

FRET and template extension reactions utilize a primer labeled with one member of a donor/acceptor pair and a nucleotide labeled with the other member of the donor/acceptor pair. Prior to incorporation of the labeled nucleotide into the primer during a template-dependent extension reaction, the donor and acceptor are spaced far enough apart that energy transfer cannot occur. However, if the labeled nucleotide is incorporated into the primer and the spacing is sufficiently close, then energy transfer occurs and can be detected. These methods are particularly useful in conducting single base pair extension reactions in the detection of single nucleotide polymorphisms and are described in U.S. Pat. No. 5,945,283 and PCT Publication WO 97/22719.

With molecular beacons, a change in conformation of the probe as it hybridizes to a complementary region of the amplified product results in the formation of a detectable signal. The probe itself includes two sections: one section at the 5′ end and the other section at the 3′ end. These sections flank the section of the probe that anneals to the probe binding site and are complementary to one another. One end section is typically attached to a reporter dye and the other end section is usually attached to a quencher dye. In solution, the two end sections can hybridize with each other to form a hairpin loop. In this conformation, the reporter and quencher dye are in sufficiently close proximity that fluorescence from the reporter dye is effectively quenched by the quencher dye. Hybridized probe, in contrast, results in a linearized conformation in which the extent of quenching is decreased. Thus, by monitoring emission changes for the two dyes, it is possible to indirectly monitor the formation of amplification product. Probes of this type and methods of their use are described further, for example, by Piatek et al., 1998, Nat. Biotechnol. 16:359-63; Tyagi, and Kramer, 1996, Nat. Biotechnology 14:303-308; and Tyagi, et al., 1998, Nat. Biotechnol. 16:49-53 (1998).

The Scorpion detection method is described, for example, by Thelwell et al. 2000, Nucleic Acids Research, 28:3752-3761 and Solinas et al., 2001, “Duplex Scorpion primers in SNP analysis and FRET applications” Nucleic Acids Research 29:20. Scorpion primers are fluorogenic PCR primers with a probe element attached at the 5′-end via a PCR stopper. They are used in real-time amplicon-specific detection of PCR products in homogeneous solution. Two different formats are possible, the “stem-loop” format and the “duplex” format. In both cases the probing mechanism is intramolecular. The basic elements of Scorpions in all formats are: (i) a PCR primer; (ii) a PCR stopper to prevent PCR read-through of the probe element; (iii) a specific probe sequence; and (iv) a fluorescence detection system containing at least one fluorophore and quencher. After PCR extension of the Scorpion primer, the resultant amplicon contains a sequence that is complementary to the probe, which is rendered single-stranded during the denaturation stage of each PCR cycle. On cooling, the probe is free to bind to this complementary sequence, producing an increase in fluorescence, as the quencher is no longer in the vicinity of the fluorophore. The PCR stopper prevents undesirable read-through of the probe by Taq DNA polymerase.

Invader assays (Third Wave Technologies, Madison, Wis.) are used particularly for SNP genotyping and utilize an oligonucleotide, designated the signal probe, that is complementary to the target nucleic acid (DNA or RNA) or polymorphism site. A second oligonucleotide, designated the Invader Oligo, contains the same 5′ nucleotide sequence, but the 3′ nucleotide sequence contains a nucleotide polymorphism. The Invader Oligo interferes with the binding of the signal probe to the target nucleic acid such that the 5′ end of the signal probe forms a “flap” at the nucleotide containing the polymorphism. This complex is recognized by a structure specific endonuclease, called the Cleavase enzyme. Cleavase cleaves the 5′ flap of the nucleotides. The released flap binds with a third probe bearing FRET labels, thereby forming another duplex structure recognized by the Cleavase enzyme. This time, the Cleavase enzyme cleaves a fluorophore away from a quencher and produces a fluorescent signal. For SNP genotyping, the signal probe will be designed to hybridize with either the reference (wild type) allele or the variant (mutant) allele. Unlike PCR, there is a linear amplification of signal with no amplification of the nucleic acid. Further details sufficient to guide one of ordinary skill in the art are provided by, for example, Neri, B. P., et al., Advances in Nucleic Acid and Protein Analysis 3826:117-125, 2000) and U.S. Pat. No. 6,706,471.

Padlock probes (PLPs) are long (e.g., about 100 bases) linear oligonucleotides. The sequences at the 3′ and 5′ ends of the probe are complementary to adjacent sequences in the target nucleic acid. In the central, noncomplementary region of the PLP there is a “tag” sequence that can be used to identify the specific PLP. The tag sequence is flanked by universal priming sites, which allow PCR amplification of the tag. Upon hybridization to the target, the two ends of the PLP oligonucleotide are brought into close proximity and can be joined by enzymatic ligation. The resulting product is a circular probe molecule catenated to the target DNA strand. Any unligated probes (i.e., probes that did not hybridize to a target) are removed by the action of an exonuclease. Hybridization and ligation of a PLP requires that both end segments recognize the target sequence. In this manner, PLPs provide extremely specific target recognition.

The tag regions of circularized PLPs can then be amplified and resulting amplicons detected. For example, TaqMan® real-time PCR can be carried out to detect and quantify the amplicon. The presence and amount of amplicon can be correlated with the presence and quantity of target sequence in the sample. For descriptions of PLPs see, e.g., Landegren et al., 2003, Padlock and proximity probes for in situ and array-based analyses: tools for the post-genomic era, Comparative and Functional Genomics 4:525-30; Nilsson et al., 2006, Analyzing genes using closing and replicating circles Trends Biotechnol. 24:83-8; Nilsson et al., 1994, Padlock probes: circularizing oligonucleotides for localized DNA detection, Science 265:2085-8.

In particular embodiments, fluorophores that can be used as detectable labels for probes include, but are not limited to, rhodamine, cyanine 3 (Cy 3), cyanine 5 (Cy 5), fluorescein, Vic™, Liz™., Tamra™, 5-Fam™, 6-Fam™, and Texas Red (Molecular Probes). (Vic™, Liz™, Tamra™, 5-Fam™, 6-Fam™ are all available from Applied Biosystems, Foster City, Calif.).

Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670.

In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product.

In particular embodiments, combined thermal cycling and fluorescence detecting devices can be used for precise quantification of target nucleic acids. In some embodiments, fluorescent signals can be detected and displayed during and/or after one or more thermal cycles, thus permitting monitoring of amplification products as the reactions occur in “real-time.” In certain embodiments, one can use the amount of amplification product and number of amplification cycles to calculate how much of the target nucleic acid sequence was in the sample prior to amplification.

According to some embodiments, one can simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target nucleic acid.

By acquiring fluorescence over different temperatures, it is possible to follow the extent of hybridization. Moreover, the temperature-dependence of PCR product hybridization can be used for the identification and/or quantification of PCR products. Accordingly, the methods described herein encompass the use of melting curve analysis in detecting and/or quantifying amplicons. Melting curve analysis is well known and is described, for example, in U.S. Pat. Nos. 6,174,670; 6,472,156; and 6,569,627, each of which is hereby incorporated by reference in its entirety, and specifically for its description of the use of melting curve analysis to detect and/or quantify amplification products. In illustrative embodiments, melting curve analysis is carried out using a double-stranded DNA dye, such as SYBR Green, Eva Green, Pico Green (Molecular Probes, Inc., Eugene, Oreg.), ethidium bromide, and the like (see Zhu et al., 1994, Anal. Chem. 66:1941-48).

According to certain embodiments, one can employ an internal control to quantify the amplification product indicated by the fluorescent signal. See, e.g., U.S. Pat. No. 5,736,333.

In various embodiments, employing preamplification, the number of preamplification cycles is sufficient to add one or more nucleotide tags to the target nucleotide sequences, so that the relative copy numbers of the tagged target nucleotide sequences is substantially representative of the relative copy numbers of the target nucleic acids in the sample. For example, preamplification can be carried out for 2-20 cycles to introduce the sample-specific or set-specific nucleotide tags. In other embodiments, detection is carried out at the end of exponential amplification, i.e., during the “plateau” phase, or endpoint PCR is carried out. In this instance, preamplification will normalize amplicon copy number across targets and across samples. In various embodiments, preamplification and/or amplification can be carried out for about: 2, 4, 10, 15, 20, 25, 30, 35, or 40 cycles or for a number of cycles falling within any range bounded by any of these values.

### Digital Amplification

For discussions of “digital PCR” see, for example, Vogelstein and Kinzler, 1999, Proc Natl Acad Sci USA 96:9236-41; McBride et al., U.S. Patent Application Publication No. 20050252773, especially Example 5 (each of these publications are hereby incorporated by reference in their entirety, and in particular for their disclosures of digital amplification). Digital amplification methods can make use of certain-high-throughput devices suitable for digital PCR, such as microfluidic devices typically including a large number and/or high density of small-volume reaction sites (e.g., nano-volume reaction sites or reaction chambers). In illustrative embodiments, digital amplification is performed using a microfluidic device, such as the Digital Array™ microfluidic devices described below. Digital amplification can entail distributing or partitioning a sample among hundreds to thousands of reaction mixtures. These reaction mixtures can be disposed in a reaction/assay platform or microfluidic device or can exist as separate droplets, e.g, as in emulsion PCR. Methods for creating droplets having reaction component(s) and/or conducting reactions therein are described in U.S. Pat. No. 7,294,503, issued to Quake et al. (which is hereby incorporated by reference in its entirety and specifically for this description); U.S. Patent Publication No. 20100022414, published Jan. 28, 2010 (assigned to Raindance Technologies, Inc.) (which is hereby incorporated by reference in its entirety and specifically for this description); U.S. Patent Publication No. 20100092973, published on Apr. 15, 2010 (assigned to Stokes Bio Ltd.) (which is hereby incorporated by reference in its entirety and specifically for this description). Digital amplification can also be carried out using the OpenArray® Real-Time PCR System available from Applied Biosystems. In such embodiments, a limiting dilution of the sample is made across a large number of separate amplification reactions such that most of the reactions have no template molecules and give a negative amplification result. In counting the number of positive amplification results, e.g, at the reaction endpoint, one is counting the individual template molecules present in the original sample one-by-one. A major advantage of digital amplification is that the quantitation is independent of variations in the amplification efficiency—successful amplifications are counted as one molecule, independent of the actual amount of product.

In particular embodiments, the methods of the invention are employed in determining the copy number of one or more target nucleic acids in a nucleic acid sample. In specific embodiments, methods and systems described herein can be used to detect copy number variation of a target nucleic acid in the genome of a subject by analyzing the genomic DNA present in a sample derived from the subject. For example, digital amplification can be carried out to determine the relative number of copies of a target nucleic acid and a reference nucleic acid in a sample. In certain embodiments, the genomic copy number is known for the reference nucleic acid (i.e., known for the particular nucleic acid sample under analysis). Alternatively, the reference nucleic acid can be one that is normally present in two copies (and unlikely to be amplified or deleted) in a diploid genome, and the copy number in the nucleic acid sample being analyzed is assumed to be two. For example, useful reference nucleic acids in the human genome include sequences of the RNaseP, β-actin, and glyceraldehyde-3-phosphate dehydrogenase (GAPDH) genes; however, it will be appreciated the invention is not limited to a particular reference nucleic acid.

In certain embodiments, digital amplification can be carried out after preamplification of sample nucleic acids. Typically, preamplification prior to digital amplification is performed for a limited number of thermal cycles (e.g., 5 cycles, or 10 cycles). In certain embodiments, the number of thermal cycles during preamplification can range from about 4 to 15 thermal cycles, or about 4-10 thermal cycles. In specific embodiments the number of thermal cycles can be 3, 4, 5, 6, 7, 8, 9, 10, 11, 12, 13, 14, 15, or more than 15. As those of skill in the art will appreciate, two or more cycles of the tagging amplification methods described above is sufficient to produce tagged target nucleotide sequence(s). When performing digital amplification for copy number determination, at least one target nucleotide sequence and at least one reference nucleotide sequence can be tagged. In certain embodiments, this amplification can be continued for a suitable number of cycles for a typical preamplification step, rendering a separate preamplification step unnecessary. Alternatively, different primers, such as, for example, tag-specific primers could be contacted with the tagged target and reference nucleotide sequences and preamplification carried out. For ease of discussion, the term “preamplification” is used below to describe amplification performed prior to digital amplification and the products of this amplification are termed “amplicons.”

In particular embodiments, preamplification reactions preferably provide quantitative amplification of the nucleic acids in the reaction mixture. That is, the relative number (ratio) of the target and reference amplicons should reflect the relative number (ratio) of target and reference nucleic acids in the nucleic acids being amplified. Methods for quantitative amplification are known in the art. See, e.g., Arya et al., 2005, Basic principles of real-time quantitative PCR, Expert Rev Mol. Diagn. 5(2):209-19. In general, primer pairs and preamplification conditions can be selected to ensure that the amplification efficiencies tagged target and tagged reference nucleotide sequences are similar or approximately equal, in order reduce any bias in the copy number determination. The amplification efficiency of any pair of primers can be easily determined using routine techniques (see e.g., Furtado et al., “Application of real-time quantitative PCR in the analysis of gene expression.” DNA amplification: Current Technologies and Applications. Wymondham, Norfolk, UK: Horizon Bioscience p. 131-145 (2004)). If the target and reference nucleotide sequences are tagged with the same tags, under suitable conditions, tag-specific primers can amplify both target and reference nucleotide sequences with similar or approximately equal amplification efficiencies. Further, limiting the number of preamplification cycles (typically to less than 15, usually 10 or less than 10, more usually about 5) greatly mitigates any differences in efficiency, such that the typical differences are likely to have an insignificant effect on the results.

Thus, following preamplification and distribution of the preamplified target and reference amplicons into separate digital amplification mixtures, a proportional number of amplicons corresponding to each sequence will be distributed into the mixtures. After digital amplification, the ratio of target and reference amplification products reflects the original ratio. Therefore, one can determine the number of reaction mixtures containing amplification product derived from the target amplicon and determine the number of reaction mixtures containing amplification product derived from the reference amplicon; and the ratio of these numbers provides the copy number of the target nucleic acid (e.g., the tagged target nucleotide sequence) relative to the reference nucleic acid (e.g., the tagged reference nucleotide sequence).

Generally, in digital amplification, identical (or substantially similar) amplification reactions are run on a nucleic acid sample, such as genomic DNA. The number of individual reactions for a given nucleic acid sample may vary from about 2 to over 1,000,000. Typically, the number of reactions performed on a sample is about 100 or greater, more typically about 200 or greater, and even more typically about 300 or greater. Larger scale digital amplification can also be performed in which the number of reactions performed on a sample is about 500 or greater, about 700 or greater, about 765 or greater, about 1,000 or greater, about 2,500 or greater, about 5,000 or greater, about 7,500 or greater, or about 10,000 or greater. The number of reactions performed may also be significantly higher, such up to about 25,000, up to about 50,000, up to about 75,000, up to about 100,000, up to about 250,000, up to about 500,000, up to about 750,000, up to about 1,000,000, or even greater than 1,000,000 assays per genomic sample.

In particular embodiments, the quantity of nucleic acid subjected to digital amplification is generally selected such that, when distributed into discrete reaction mixtures, each individual amplification reaction is expected to include one or fewer amplifiable nucleic acids. One of skill in the art can determine the concentration of target amplicon(s) produced as described above and calculate an appropriate amount for use in digital amplification. More conveniently, a set of serial dilutions of the target amplicon(s) can be tested. For example, the 12.765 Digital Array™ IFC (commercially available from Fluidigm Corp.) allows 12 different dilutions to be tested simultaneously. Optionally, a suitable dilution can be determined by generating a linear regression plot. For the optimal dilution, the line should be straight and pass through the origin. Subsequently the concentration of the original samples can be calculated from the plot.

The appropriate quantity of target and reference amplicon(s) can be distributed into discrete locations or reaction wells or chambers such that each reaction includes, for example, an average of no more than about one target amplicon and one reference amplicon per volume. The target and reference amplicon(s) can be combined with reagents selected for quantitative or nonquantitative amplification, prior to distribution or after.

Following distribution, the reaction mixtures are subjected to amplification to identify those reaction mixtures that contain a target and/or amplicon. Any amplification method can be employed, but conveniently, PCR is used, e.g., real-time PCR or endpoint PCR. This amplification can employ any primers capable of amplifying the target and/or reference amplicon(s). Digital amplification can be can be carried out wherein the target and reference amplicons are distributed into sets of reaction mixtures for detection of amplification products derived from one type of amplicon, either target or reference amplicons. In such embodiments, two sets of reaction mixtures, a target set and a reference set, could have distinct primer pairs, one for amplifying target amplicons, and one for amplifying reference amplicons could be used. Amplification product could be detected, for example, using a universal probe, such as SYBR Green, or target- and reference-specific probes, which could be included in all digital amplification mixtures.

The concentration of any target or reference amplicon (copies/4) is correlated with the number of reaction mixtures that are positive (i.e., amplification product-containing) for that particular amplicon. See copending U.S. application Ser. No. 12/170,414, entitled “Method and Apparatus for Determining Copy Number Variation Using Digital PCR,” which is incorporated by reference for all purposes, and, in particular, for analysis of digital PCR results. Also see Dube et al., 2008, “Mathematical Analysis of Copy Number Variation in a DNA Sample Using Digital PCR on a Nanofluidic Device” PLoS ONE 3(8): e2876. doi:10.1371/journal.pone.0002876, which is incorporated by reference for all purposes and, in particular, for analysis of digital PCR results.

### DNA Sequencing

Many current DNA sequencing techniques rely on “sequencing by synthesis.” These techniques entail library creation, massively parallel PCR amplification of library molecules, and sequencing. Library creation starts with conversion of sample nucleic acids to appropriately sized fragments, ligation of adaptor sequences onto the ends of the fragments, and selection for molecules properly appended with adaptors. The presence of the adaptor sequences on the ends of the library molecules enables amplification of random-sequence inserts. The above-described methods for tagging nucleotide sequences can be substituted for ligation, to introduce adaptor sequences.

In particular embodiments, the number of library DNA molecules produced in the massively parallel PCR step is low enough that the chance of two molecules associating with the same substrate, e.g. the same bead (in 454 DNA sequencing) or the same surface patch (in Solexa DNA sequencing) is low, but high enough so that the yield of amplified sequences is sufficient to provide a high throughput. After suitable adaptor sequences are introduced, digital PCR can be employed to calibrate the number of library DNA molecules prior to sequencing by synthesis.

The methods of the invention can include subjecting at least one target amplicon to DNA sequencing using any available DNA sequencing method. In particular embodiments, a plurality of target amplicons is sequenced using a high throughput sequencing method. Such methods typically use an in vitro cloning step to amplify individual DNA molecules. Emulsion PCR (emPCR) isolates individual DNA molecules along with primer-coated beads in aqueous droplets within an oil phase. PCR produces copies of the DNA molecule, which bind to primers on the bead, followed by immobilization for later sequencing. emPCR is used in the methods by Marguilis et al. (commercialized by 454 Life Sciences, Branford, Conn.), Shendure and Porreca et al. (also known as “polony sequencing”) and SOLiD sequencing, (Applied Biosystems Inc., Foster City, Calif.). See M. Margulies, et al. (2005) “Genome sequencing in microfabricated high-density picolitre reactors” Nature 437: 376-380; J. Shendure, et al. (2005) “Accurate Multiplex Polony Sequencing of an Evolved Bacterial Genome” Science 309 (5741): 1728-1732. In vitro clonal amplification can also be carried out by “bridge PCR,” where fragments are amplified upon primers attached to a solid surface. Braslaysky et al. developed a single-molecule method (commercialized by Helicos Biosciences Corp., Cambridge, Mass.) that omits this amplification step, directly fixing DNA molecules to a surface. I. Braslaysky, et al. (2003) “Sequence information can be obtained from single DNA molecules” Proceedings of the National Academy of Sciences of the United States of America 100: 3960-3964.

DNA molecules that are physically bound to a surface can be sequenced in parallel. “Sequencing by synthesis,” like dye-termination electrophoretic sequencing, uses a DNA polymerase to determine the base sequence. Reversible terminator methods (commercialized by Illumina, Inc., San Diego, Calif. and Helicos Biosciences Corp., Cambridge, Mass.) use reversible versions of dye-terminators, adding one nucleotide at a time, and detect fluorescence at each position in real time, by repeated removal of the blocking group to allow polymerization of another nucleotide. “Pyrosequencing” also uses DNA polymerization, adding one nucleotide at a time and detecting and quantifying the number of nucleotides added to a given location through the light emitted by the release of attached pyrophosphates (commercialized by 454 Life Sciences, Branford, Conn.). See M. Ronaghi, et al. (1996). “Real-time DNA sequencing using detection of pyrophosphate release” Analytical Biochemistry 242: 84-89.

### Labeling Strategies

Any suitable labeling strategy can be employed in the methods of the invention. Where the assay mixture is aliquoted, and each aliquot is analyzed for presence of a single amplification product, a universal detection probe can be employed in the amplification mixture. In particular embodiments, real-time PCR detection can be carried out using a universal qPCR probe. Suitable universal qPCR probes include double-stranded DNA dyes, such as SYBR Green, Pico Green (Molecular Probes, Inc., Eugene, Oreg.), Eva Green (Biotinum), ethidium bromide, and the like (see Zhu et al., 1994, Anal. Chem. 66:1941-48). Suitable universal qPCR probes also include sequence-specific probes that bind to a nucleotide sequence present in all amplification products. Binding sites for such probes can be conveniently introduced into the tagged target nucleic acids during amplification.

Alternatively, one or more target-specific qPCR probes (i.e., specific for a target nucleotide sequence to be detected) is employed in the amplification mixtures to detect amplification products. Target-specific probes could be useful, e.g., when only a few target nucleic acids are to be detected in a large number of samples. For example, if only three targets were to be detected, a target-specific probe with a different fluorescent label for each target could be employed. By judicious choice of labels, analyses can be conducted in which the different labels are excited and/or detected at different wavelengths in a single reaction. See, e.g., Fluorescence Spectroscopy (Pesce et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2nd ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook of Fluorescent Probes and Research Chemicals, Molecular Probes, Eugene (1992).

An “indirect” labeling strategy can be employed wherein the amplicon to be detection includes a nucleotide tag or when a primer in a preamplification or amplification mixture includes such a tag. In this case, an amplification mixture can included a labeled (e.g., fluorescently labeled) nucleotide tag-specific primer.

Other labeling strategies that can be employed in the methods described herein include, e.g., that described in U.S. Pat. No. 7,615,620, issued Nov. 10, 2009 to Robinson (assigned to KBiosciences Ltd.), which discloses a FRET detection system for an amplification process that employs at least two single-labeled oligonucleotide sequences of differing Tm that hybridize to one another in free solution to form a fluorescent quenched pair, that upon introduction of a complementary sequence to one or both sequences generates a measurable signal, one of the sequences being of a Tm that is below the Ta of the PCR process, the other not being below the Ta of the PCR process. This patent is incorporated herein in its entirety and for this disclosure.

International Publication No. WO/1997/032044, published Sep. 4, 1997 (assigned to E.I. Du Pont De Nemours And Company) describes a detection probe the is present throughout an amplification reaction but does not participate in the reaction in that it is not extended. The probe contains sequence complementary to the replicated nucleic acid target for capture of the target by hybridization. Additionally, the probe or target contains at least one reactive ligand to permit immobilization or reporting of the probe/target hybrid. Such labeling systems can be employed in the methods described herein. Accordingly, this publication is incorporated by reference herein in its entirety and for its disclosure such labeling systems.

Additional labeling strategies useful in the methods described herein are found in U.S. Pat. No. 5,928,862, issued Jul. 27, 1999 to Morrison (assigned to Amoco Corp.), which discloses a competitive homogeneous assay and is incorporated by reference herein in its entirety and for this disclosure.

U.S. Pat. No. 6,103,476, issued Aug. 15, 2000 to Tyagi et al. (assigned to The Public Health Research Institute of the City of New York, Inc.) describes unimolecular and bimolecular hybridization probes that include a target complement sequence, an affinity pair holding the probe in a closed conformation in the absence of target sequence, and either a label pair that interacts when the probe is in the closed conformation or, for certain unimolecular probes, a non-interactive label. Hybridization of the target and target complement sequences shifts the probe to an open conformation. The shift is detectable due to reduced interaction of the label pair or by detecting a signal from a non-interactive label. Certain unimolecular probes can discriminate between target and non-target sequences differing by as little as one nucleotide. Such labeling systems can be employed in the methods described herein. Accordingly, this patent is incorporated by reference herein in its entirety and for its disclosure such labeling systems.

In some embodiments, significant modifications to the sugar linkage of probes (including but not limited to 2′ O-methyl, 2′ O-Fluoro) or substitution of the phosphodiester linkage (including but not limited to phosphorothioate or amino moieties) are envisaged. Screening in both a tiling approach or roughly speading detection to multiple common probe binding sites in the test and reference loci provides the benefit that detection and screening can be spread across either large loci or across even chromosomes. The intent of this approach is to increase the number of assays to reduce biological variability and simultaneously increase the number of sampled molecules which reduces statistical variation. Target search for common motifs across chromosomal regions permitting uniform 5′ nuclease mediated probe selection has already been performed.

### Removal of Undesired Reaction Components

It will be appreciated that reactions involving complex mixtures of nucleic acids in which a number of reactive steps are employed can result in a variety of unincorporated reaction components, and that removal of such unincorporated reaction components, or reduction of their concentration, by any of a variety of clean-up procedures can improve the efficiency and specificity of subsequently occurring reactions. For example, it may be desirable, in some embodiments, to remove, or reduce the concentration of preamplification primers prior to carrying out the amplification steps described herein.

In certain embodiments, the concentration of undesired components can be reduced by simple dilution. For example, preamplified samples can be diluted about 2-, 5-, 10-, 50-, 100-, 500-, 1000-fold prior to amplification to improve the specificity of the subsequent amplification step.

In some embodiments, undesired components can be removed by a variety of enzymatic means. Alternatively, or in addition to the above-described methods, undesired components can be removed by purification. For example, a purification tag can be incorporated into any of the above-described primers to facilitate purification of the tagged target nucleotides.

In particular embodiments, clean-up includes selective immobilization of the desired nucleic acids. For example, desired nucleic acids can be preferentially immobilized on a solid support. In an illustrative embodiment, an affinity moiety, such as biotin (e.g., photo-biotin), is attached to desired nucleic acid, and the resulting biotin-labeled nucleic acids immobilized on a solid support comprising an affinity moiety-binder such as streptavidin. Immobilized nucleic acids can be queried with probes, and non-hybridized and/or non-ligated probes removed by washing (See, e.g., Published P.C.T. Application WO 03/006677 and U.S. Ser. No. 09/931,285.) Alternatively, immobilized nucleic acids can be washed to remove other components and then released from the solid support for further analysis. This approach can be used, for example, in recovering target amplicons from amplification mixtures after the addition of primer binding sites for DNA sequencing. In particular embodiments, an affinity moiety, such as biotin, can be attached to an amplification primer such that amplification produces an affinity moiety-labeled (e.g., biotin-labeled) amplicon.

### Microfluidic Devices

In certain embodiments, any of the methods of the invention can be carried out using a microfluidic device. In illustrative embodiments, the device is a matrix-type microfluidic device is one that allows the simultaneous combination of a plurality of substrate solutions with reagent solutions in separate isolated reaction chambers. It will be recognized, that a substrate solution can comprise one or a plurality of substrates and a reagent solution can comprise one or a plurality of reagents. For example, the microfluidic device can allow the simultaneous pair-wise combination of a plurality of different amplification primers and samples. In certain embodiments, the device is configured to contain a different combination of primers and samples in each of the different chambers. In various embodiments, the number of separate reaction chambers can be greater than 50, usually greater than 100, more often greater than 500, even more often greater than 1000, and sometimes greater than 5000, or greater than 10,000.

In particular embodiments, the matrix-type microfluidic device is a Dynamic Array™ microfluidic device. A Dynamic Array™ microfluidic device is a matrix-type microfluidic device designed to isolate pair-wise combinations of samples and reagents (e.g., amplification primers, detection probes, etc.) and suited for carrying out qualitative and quantitative PCR reactions including real-time quantitative PCR analysis. In some embodiments, the DA microfluidic device is fabricated, at least in part, from an elastomer. DA microfluidic devices are described in PCT publication W005107938A2 (Thermal Reaction Device and Method For Using The Same) and US Pat. Publication US20050252773A1, both incorporated herein by reference in their entireties for their descriptions of DA microfluidic devices. DA microfluidic devices may incorporate high-density matrix designs that utilize fluid communication vias between layers of the microfluidic device to weave control lines and fluid lines through the device and between layers. By virtue of fluid lines in multiple layers of an elastomeric block, high density reaction cell arrangements are possible. Alternatively DA microfluidic devices may be designed so that all of the reagent and sample channels are in the same elastomeric layer, with control channels in a different layer.

Although the DA microfluidic devices described above in WO 05/107938 are well suited for conducting the methods described herein, the invention is not limited to any particular device or design. Any device that partitions a sample and/or allows independent pair-wise combinations of reagents and sample may be used. U.S. Patent Publication No. 20080108063 (which is hereby incorporated by reference it its entirety) includes a diagram illustrating the 48.48 Dynamic Array™ IFC (Integrated Fluidic Circuit), a commercially available device available from Fluidigm Corp. (South San Francisco Calif.). It will be understood that other configurations are possible and contemplated such as, for example, 48×96; 96×96; 30×120; etc.

In specific embodiments, the microfluidic device can be a Digital Array™ microfluidic device, which is adapted to perform digital amplification. Such devices can have integrated channels and valves that partition mixtures of sample and reagents into nanolitre volume reaction chambers. In some embodiments, the Digital Array™ microfluidic device is fabricated, at least in part, from an elastomer. Illustrative Digital Array™ microfluidic devices are described in copending U.S. Applications owned by Fluidigm, Inc., such as U.S. application Ser. No. 12/170,414, entitled “Method and Apparatus for Determining Copy Number Variation Using Digital PCR.” One illustrative embodiment has 12 input ports corresponding to 12 separate sample inputs to the device. The device can have 12 panels, and each of the 12 panels can contain 765 6 mL reaction chambers with a total volume of 4.59 μL per panel. Microfluidic channels can connect the various reaction chambers on the panels to fluid sources. Pressure can be applied to an accumulator in order to open and close valves connecting the reaction chambers to fluid sources. In illustrative embodiments, 12 inlets can be provided for loading of the sample reagent mixture. 48 inlets can be used to provide a source for reagents, which are supplied to the biochip when pressure is applied to accumulator. Additionally, two or more inlets can be provided to provide hydration to the biochip. Hydration inlets are in fluid communication with the device to facilitate the control of humidity associated with the reaction chambers. As will be understood to one of skill in the art, some elastomeric materials that can utilized in the fabrication of the device are gas permeable, allowing evaporated gases or vapor from the reaction chambers to pass through the elastomeric material into the surrounding atmosphere. In a particular embodiment, fluid lines located at peripheral portions of the device provide a shield of hydration liquid, for example, a buffer or master mix, at peripheral portions of the biochip surrounding the panels of reaction chambers, thus reducing or preventing evaporation of liquids present in the reaction chambers. Thus, humidity at peripheral portions of the device can be increased by adding a volatile liquid, for example water, to hydration inlets. In a specific embodiment, a first inlet is in fluid communication with the hydration fluid lines surrounding the panels on a first side of the biochip and the second inlet is in fluid communication with the hydration fluid lines surrounding the panels on the other side of the biochip.

While the Digital Array™ microfluidic devices are well-suited for carrying out the digital amplification methods described herein, one of ordinary skill in the art would recognize many variations and alternatives to these devices. The microfluidic device which is the 12.765 Digital Array™ commercially available from Fluidigm Corp. (South San Francisco, Calif.), includes 12 panels, each having 765 reaction chambers with a volume of 6 mL per reaction chamber. However, this geometry is not required for the digital amplification methods described herein. The geometry of a given Digital Array™ microfluidic device will depend on the particular application. Additional description related to devices suitable for use in the methods described herein is provided in U.S. Patent Application Publication No. 2005/0252773, incorporated herein by reference for its disclosure of Digital Array™ microfluidic devices.

In certain embodiments, the methods described herein can be performed using a microfluidic device that provides for recovery of reaction products. Such devices are described in detail in copending U.S. Application No. 61/166,105, filed Apr. 2, 2009, which is hereby incorporated by reference in its entirety and specifically for its description of microfluidic devices that permit reaction product recovery and related methods. For example, the digital PCR method for calibrating DNA samples prior to sequencing can be preformed on such devices, permitting recovery of amplification products, which can then serve as templates for DNA sequencing.

Embodiments using a microfluidic device that provides for recovery of reaction products provide a system suitable for PCR sample preparation that features reduced cost, time, and labor in the preparation of amplicon libraries from an input DNA template. In a typical use case, the first amplification will be used to generate libraries for next-generation sequencing. Utilizing embodiments of the present invention, samples and encoded primers are combined with amplicon-specific (AS) primers to create a mixture that is suitable for desired reactions. Based on an M×N architecture of the microfluidic device, each of the M samples is combined with each of the N AS primers (i.e., assays) to form M×N pairwise combinations. That is, one reaction site is provided for each sample and assay pair. After the completion of the reaction (e.g., PCR), the reaction products are recovered from the system, typically using a harvest reagent that flows through the microfluidic device. In a specific embodiment, reaction products associated with each sample are recovered in a separate reaction pool, enabling further processing or study of the pool containing a given sample reacted with each of the various assays.

Thus, in embodiments described herein, a microfluidic device is provided in which independent sample inputs are combined with primer inputs in an M×N array configuration. Thus, each reaction is a unique combination of a particular sample and a particular primer. As described more fully throughout the present specification, samples are loaded into sample chambers in the microfluidic device through sample input lines arranged as columns in one implementation. AS primers or assays are loaded into assay chambers in the microfluidic device through assay input lines arranged as rows crossing the columns. The sample chambers and the assay chambers are in fluidic isolation during loading. After the loading process is completed, an interface valve operable to obstruct a fluid line passing between pairs of sample and assay chambers is opened to enable free interface diffusion of the pairwise combinations of samples and assays. Precise mixture of the samples and assays enables reactions to occur between the various pairwise combinations, producing a reaction product including a set of specific PCR reactions for which each sample has been effectively coded with a unique barcode. The reaction products are harvested and can then be used for subsequent sequencing processes. The terms “assay” and “sample” as used herein are descriptive of particular uses of the devices in some embodiments. However, the uses of the devices are not limited to the use of “sample(s)” and “assay(s)” in all embodiments. For example, in other embodiments, “sample(s)” may refer to “a first reagent” or a plurality of “first reagents” and “assay(s)” may refer to “a second reagent” or a plurality of “second reagents.” The M×N character of the devices enable the combination of any set of first reagents to be combined with any set of second reagents.

According to one particular process implemented using an embodiment of the present invention, after 25 cycles of PCR, the reaction products from the M×N pairwise combinations will be recovered from the microfluidic device in discrete pools, one for each of the M samples. Typically, the discrete pools are contained in a sample input port provided on the carrier. In some processes, the reaction products may be harvested on a “per amplicon” basis for purposes of normalization. Utilizing embodiments of the present invention, it is possible to achieve results (for replicate experiments assembled from the same input solutions of samples and assays) for which the copy number of amplification products varies by no more than ±25% within a sample and no more than ±25% between samples. Thus, the amplification products recovered from the microfluidic device will be representative of the input samples as measured by the distribution of specific known genotypes. Preferably, output sample concentration will be greater than 2,000 copies/amplicon/microliter and recovery of reaction products will be performed in less than two hours.

Applications in which embodiments of the present invention can be used include sequencer-ready amplicon preparation and long-range PCR amplicon library production. For the sequencer-ready amplicon preparation, multiple-forward primer and 3-primer combination protocols can be utilized.

The methods described herein may use microfluidic devices with unit cells with dimensions on the order of several hundred microns, for example unit cells with dimension of 500×500 μm, 525×525 μm, 550×550 μm, 575×575 μm, 600×600 μm, 625×625 μm, 650×650 μm, 675×675, μm, 700×700 μm, or the like. The dimensions of the sample chambers and the assay chambers are selected to provide amounts of materials sufficient for desired processes while reducing sample and assay usage. As examples, sample chambers can have dimensions on the order of 100-400 μm in width×200-600 μm in length×100-500 μm in height. For example, the width can be 100 μm, 125 μm, 150 μm, 175 μm, 200 μm, 225 μm, 250 μm, 275 μm, 300 μm, 325 μm, 350 μm, 375 μm, 400 μm, or the like. For example, the length can be 200 μm, 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, or the like. For example, the height can be 100 μm, 125 μm, 150 μm, 175 μm, 200 μm, 225 μm, 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, or the like. Assay chambers can have similar dimensional ranges, typically providing similar steps sizes over smaller ranges than the smaller chamber volumes. In some embodiments, the ratio of the sample chamber volume to the assay chamber volume is about 5:1, 10:1, 15:1, 20:1, 25:1, or 30:1. Smaller chamber volumes than the listed ranges are included within the scope of the invention and are readily fabricated using microfluidic device fabrication techniques.

Higher density microfluidic devices will typically utilize smaller chamber volumes in order to reduce the footprint of the unit cells. In applications for which very small sample sizes are available, reduced chamber volumes will facilitate testing of such small samples.

In some embodiments, reaction products are recovered by dilation pumping. Dilation pumping provides benefits not typically available using conventional techniques. For example, dilation pumping enables for a slow removal of the reaction products from the microfluidic device. In an exemplary embodiment, the reaction products are recovered at a fluid flow rate of less than 100 μl per hour. In this example, for 48 reaction products distributed among the reaction chambers in each column, with a volume of each reaction product of about 1.5 μl, removal of the reaction products in a period of about 30 minutes, will result in a fluid flow rate of 72 μl/hour. (i.e., 48*1.5/0.5 hour). In other embodiments, the removal rate of the reaction products is performed at a rate of less than 90 μl/hr, 80 μl/hr, 70 μl/hr, 60 μl/hr, 50 μl/hr, 40 μl/hr, 30 μl/hr, 20 μl/hr, 10 μl/hr, 9 μl/hr, less than 8 μl/hr, less than 7 μl/hr, less than 6 μl/hr, less than 5 μl/hr, less than 4 μl/hr, less than 3 μl/hr, less than 2 μl/hr, less than 1 μl/hr, or less than 0.5 μl/hr.

Dilation pumping results in clearing of substantially a high percentage and potentially all the reaction products present in the microfluidic device. Some embodiments remove more than 75% of the reaction products present in the reaction chambers (e.g., sample chambers) of the microfluidic device. As an example, some embodiments remove more than 80%, 85%, 90%, 92%, 95%, 96%, 97%, 98%, or 99% of the reaction products present in the reaction chambers.

Another microfluidic device that can be employed in the methods described herein is disclosed in PCT Pub. No. WO/2009/059430, published May 14, 2009 (Hansen and Tropini), which is incorporated herein by reference in its entirety and, specifically, for it's description of microfluidic devices, their production, and use. This microfluidic device includes a plurality of reaction chambers in fluid communication with a flow channel formed in an elastomeric substrate, a vapor barrier for preventing evaporation from the plurality of reaction chambers, and a continuous phase fluid for isolation of each of the plurality of reaction chambers.

Fabrication methods using elastomeric materials and methods for design of devices and their components have been described in detail in the scientific and patent literature. See, e.g., Unger et al. (2000) Science 288:113-116; U.S. Pat. No. 6,960,437 (Nucleic acid amplification utilizing microfluidic devices); U.S. Pat. No. 6,899,137 (Microfabricated elastomeric valve and pump systems); U.S. Pat. No. 6,767,706 (Integrated active flux microfluidic devices and methods); U.S. Pat. No. 6,752,922 (Microfluidic chromatography); U.S. Pat. No. 6,408,878 (Microfabricated elastomeric valve and pump systems); U.S. Pat. No. 6,645,432 (Microfluidic systems including three-dimensionally arrayed channel networks); U.S. Patent Application Publication Nos. 2004/0115838; 2005/0072946; 2005/0000900; 2002/0127736; 2002/0109114; 2004/0115838; 2003/0138829; 2002/0164816; 2002/0127736; and 2002/0109114; PCT Publication Nos. WO 2005/084191; WO 05/030822A2; and WO 01/01025; Quake & Scherer, 2000, “From micro to nanofabrication with soft materials” Science 290: 1536-40; Unger et al., 2000, “Monolithic microfabricated valves and pumps by multilayer soft lithography” Science 288:113-116; Thorsen et al., 2002, “Microfluidic large-scale integration” Science 298:580-584; Chou et al., 2000, “Microfabricated Rotary Pump” Biomedical Microdevices 3:323-330; Liu et al., 2003, “Solving the “world-to-chip” interface problem with a microfluidic matrix” Analytical Chemistry 75, 4718-23, Hong et al, 2004, “A nanoliter-scale nucleic acid processor with parallel architecture” Nature Biotechnology 22:435-39.

According to certain embodiments described herein, the detection and/or quantification of one or more target nucleic acids from one or more samples may generally be carried out on a microfluidic device by obtaining a sample, optionally pre-amplifying the sample, and distributing the optionally pre-amplified sample, or aliquots thereof, into reaction chambers of a microfluidic device containing the appropriate buffers, primers, optional probe(s), and enzyme(s), subjecting these mixtures to amplification, and querying the aliquots for the presence of amplified target nucleic acids. The sample aliquots may have a volume of less than 1 picoliter or, in various embodiments, in the range of about 1 picoliter to about 500 nanoliters, in a range of about 2 picoliters to about 50 picoliters, in a range of about 5 picoliters to about 25 picoliters, in the range of about 100 picoliters to about 20 nanoliters, in the range of about 1 nanoliter to about 20 nanoliters, and in the range of about 5 nanoliters to about 15 nanoliters. In many embodiments, sample aliquots account for the majority of the volume of the amplification mixtures. Thus, amplification mixtures can have a volume of less than 1 picoliter or, in various embodiments about 2, about 5 about 7, about 10, about 15, about 20, about 25, about 50, about 100, about 250, about 500, and about 750 picoliters; or about 1, about 2, about 5, about 7, about 15, about 20, about 25, about 50, about 250, and about 500 nanoliters. The amplification mixtures can also have a volume within any range bounded by any of these values (e.g., about 2 picoliters to about 50 picoliters).

In certain embodiments, multiplex detection is carried out in individual amplification mixture, e.g., in individual reaction chambers of a microfluidic device, which can be used to further increase the number of samples and/or targets that can be analyzed in a single assay or to carry out comparative methods, such as comparative genomic hybridization (CGH). In various embodiments, up to 2, 3, 4, 5, 6, 7, 8, 9, 10, 50, 100, 500, 1000, 5000, 10000 or more amplification reactions are carried out in each individual reaction chamber.

In specific embodiments, the assay usually has a dynamic range of at least 3 orders of magnitude, more often at least 4, at least 5, at least 6, at least 7, or at least 8 orders of magnitude.

### Data Output and Analysis

In certain embodiments, when the methods of the invention are carried out on a matrix-type microfluidic device, the data can be output as a heat matrix (also termed “heat map”). In the heat matrix, each square, representing a reaction chamber on the DA matrix, has been assigned a color value which can be shown in gray scale, but is more typically shown in color. In gray scale, black squares indicate that no amplification product was detected, whereas white squares indicate the highest level of amplification produce, with shades of gray indicating levels of amplification product in between. In a further aspect, a software program may be used to compile the data generated in the heat matrix into a more reader-friendly format.

### Applications

The methods of the invention are applicable to any technique aimed at detecting the presence or amount of one or more target nucleic acids in a nucleic acid sample. Thus, for example, these methods are applicable to identifying the presence of particular polymorphisms (such as SNPs), alleles, or haplotypes, or chromosomal abnormalities, such as amplifications, deletions, or aneuploidy. The methods may be employed in genotyping, which can be carried out in a number of contexts, including diagnosis of genetic diseases or disorders, pharmacogenomics (personalized medicine), quality control in agriculture (e.g., for seeds or livestock), the study and management of populations of plants or animals (e.g., in aquaculture or fisheries management or in the determination of population diversity), or paternity or forensic identifications. The methods of the invention can be applied in the identification of sequences indicative of particular conditions or organisms in biological or environmental samples. For example, the methods can be used in assays to identify pathogens, such as viruses, bacteria, and fungi). The methods can also be used in studies aimed at characterizing environments or microenvironments, e.g., characterizing the microbial species in the human gut.

These methods can also be employed in determinations DNA or RNA copy number. Determinations of aberrant DNA copy number in genomic DNA is useful, for example, in the diagnosis and/or prognosis of genetic defects and diseases, such as cancer. Determination of RNA “copy number,” i.e., expression level is useful for expression monitoring of genes of interest, e.g., in different individuals, tissues, or cells under different conditions (e.g., different external stimuli or disease states) and/or at different developmental stages.

In addition, the methods can be employed to prepare nucleic acid samples for further analysis, such as, e.g., DNA sequencing.

Finally, nucleic acid samples can be tagged as a first step, prior subsequent analysis, to reduce the risk that mislabeling or cross-contamination of samples will compromise the results. For example, any physician's office, laboratory, or hospital could tag samples immediately after collection, and the tags could be confirmed at the time of analysis. Similarly, samples containing nucleic acids collected at a crime scene could be tagged as soon as practicable, to ensure that the samples could not be mislabeled or tampered with. Detection of the tag upon each transfer of the sample from one party to another could be used to establish chain of custody of the sample.

### Kits

Kits according to the invention include one or more reagents useful for practicing one or more assay methods of the invention. A kit generally includes a package with one or more containers holding the reagent(s) (e.g., primers and/or probe(s)), as one or more separate compositions or, optionally, as admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits according to the invention generally include instructions for carrying out one or more of the methods of the invention. Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), RF tags, and the like. As used herein, the term “instructions” can include the address of an internet site that provides the instructions.

It is understood that the examples and embodiments described herein are for illustrative purposes only and that various modifications or changes in light thereof will be suggested to persons skilled in the art and are to be included within the spirit and purview of this application and scope of the appended claims.

In addition, all other publications, patents, and patent applications cited herein are hereby incorporated by reference in their entirety for all purposes.

### EXAMPLES

### Example 1

### Use of Fluorescent Primers and Intercalating Dye to Generate Fluorescent PCR Signals (Real-Time, End-Point, Multiplex)

### Problem Statement:

There are many methods for the generation of fluorescent signal during PCR (and similar methods) or as end-point signal. Most systems require at least 2 non-standard modifications on probe or primers and consequently are relatively expensive. Usually every assay needs its own probe or fluorescent primer.

### Solution:

This problem can be solved by generating amplification signal by the use of a fluorescent labeled primer and an intercalating dye (“LCGreen” used as best current choice) to generate amplification specific changes in fluorescent signal.

Aspects of the Solution:

Fluorescent primers are tag specific—universal detector for any assay.

Multiplexing by different dyes on different primers.

End point and real-time analysis.

Melting analysis of product (LCGreen and primer label).

Combination with (quenched) complementary oligo to improve signal/noise ratio and possibly specificity.

Reading of baseline with same filter combination but at different temperature.

The same quenching concept may also be used for fluorescent probes. E.g. generation of ssDNA product (RCA, NASBA, asymmetric PCR) and binding of single label fluorescent probe to the product.

### Steps Provided for in the Current Method:

Combination of fluorescent primers and intercalating dye. Use of intercalating dye to quench the signal of the fluorescent dye on the primer. The quenching (and thus the fluorescent signal) will be different depending on the primer being single stranded or part of a PCR product. The signal intensity is also temperature dependent: At low temperatures (below approximately 55-70° C.), the single stranded primers Fluorescence is efficiently quenched, such that the signal is much weaker than from a primer in PCR product (positive signal for PCR product). Above ˜65 to 75° C. the single stranded primers signal is not quenched anymore, while it is quenched in the PCR product until the product is completely denatured product (negative signal for PCR product) (depending on amplicon properties at approx. 75 to 90° C.)

Makes use of different affinities of intercalating dye to single and double stranded DNA and of different distances for quenching (FRET<->contact quenching).

Reading of baseline with same filter combination but at different temperature.

### Example

### Alternative Signal Generation (Tag Specific Fluorescent Primers)

iFRET:

Signal of dye introduced by the primer is generated by excitation of SYBR, which then FRETs to the dye on the 5′-end of the PCR product. The “classical” scheme for iFRET signal generation (excitation at LC Green wavelength and reading at emission wavelength of primer-dye) did not work. It seems that the observed signal is mainly due to LCGreen itself Differences between primers with different labels was minimal.

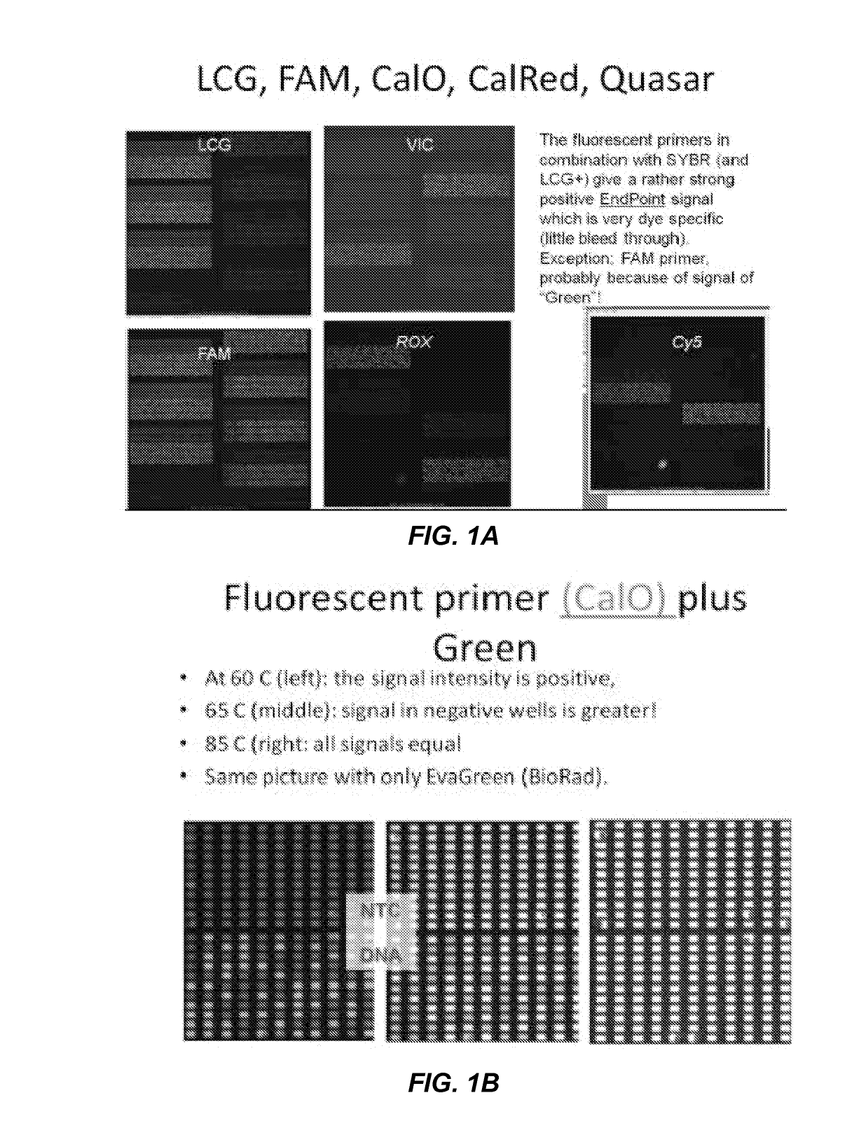

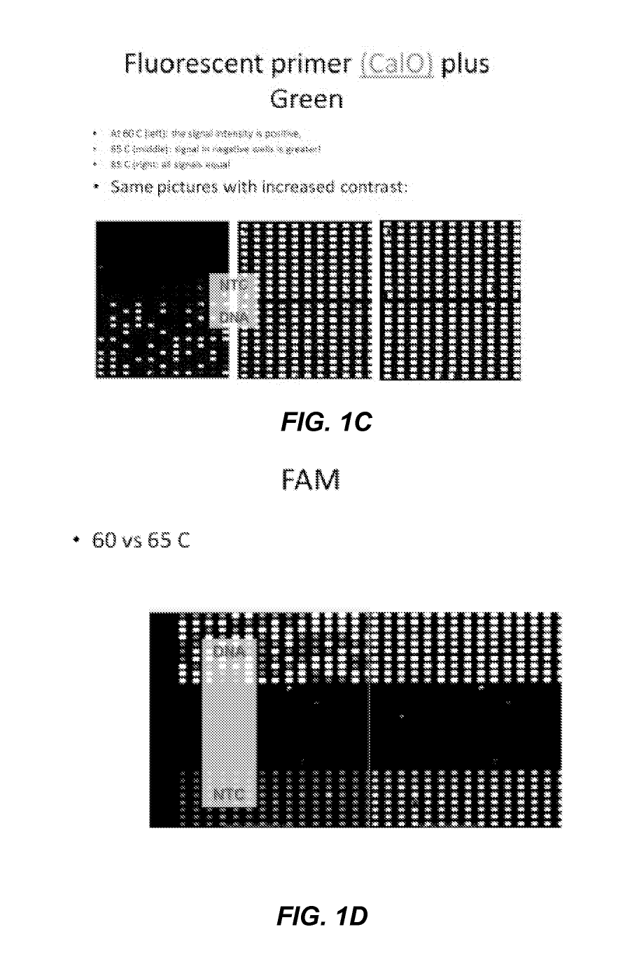

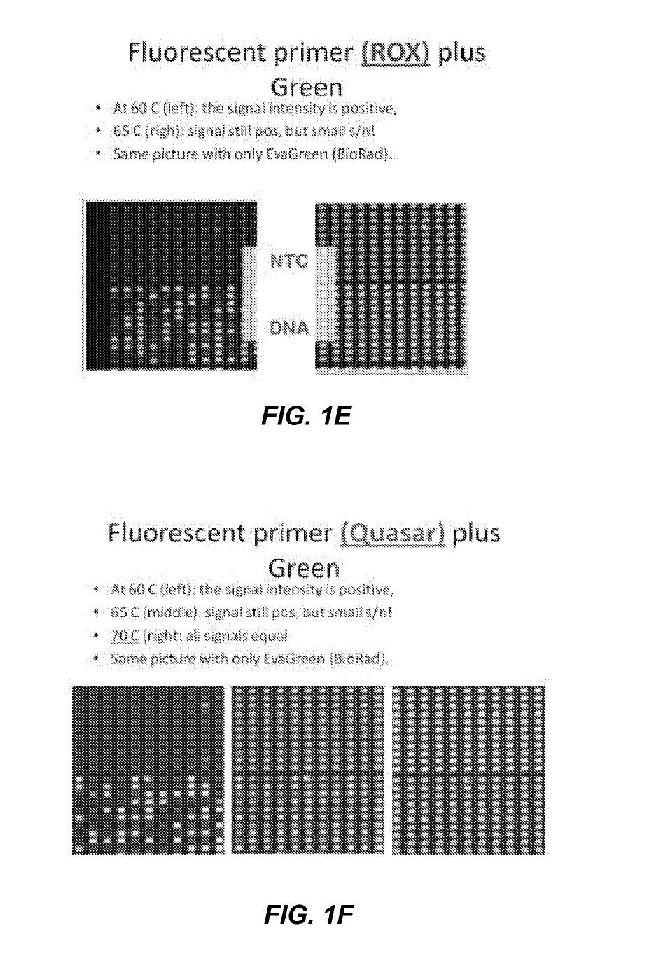

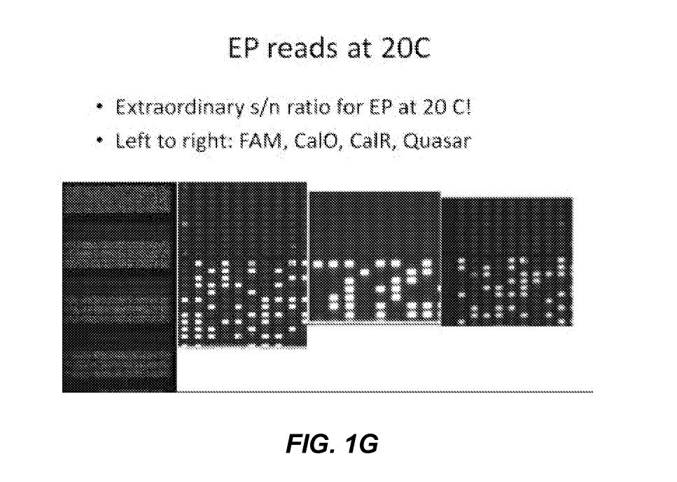

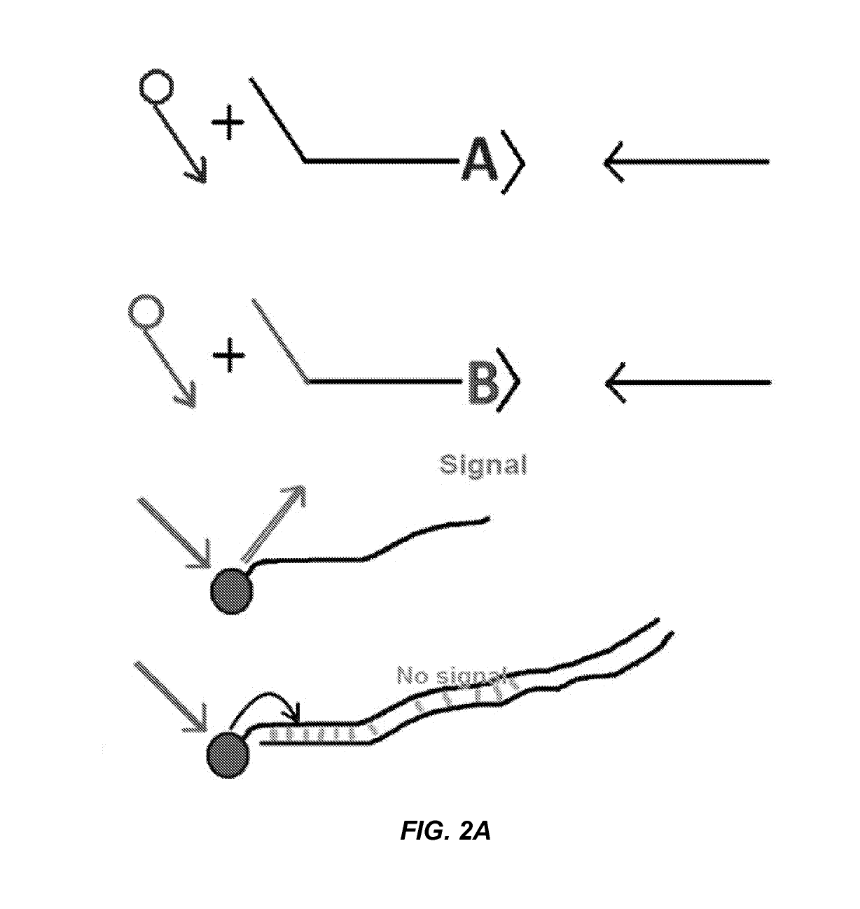

Using the same reactions as above, but reading with excitation and emission of primer-label showed surprising strong signal/noise at 60° C. Signal was even better with End Point reading at 20° C. The background signal increases between 60° C. and 70° C. in a way that the signal of PCR negative reactions is stronger than the signal of positive reactions. The results are shown in FIGS. 1A-G.

Fluorescent primer plus Quenching oligo: When fluorescent primer is incorporated into ds PCR product it can no longer be quenched by the CQ (complementary quencher). CQ has one mismatch with primer to promote efficient priming.

### Example 2

### SNP by Tagging and Universal Fluorescent Primers

### Problem:

Find a cost effective method for detection of SNPs in micro fluidic chips.

### Solution:

Perform allele specific PCR with a tag on the allele-specific Forward primers. The two variants carry distinct tags.

Matching each distinct “allele-specific” tag, a tag-specific fluorescent primer (same sequence as tag) is in the reaction (for example: allele A: FAM, allele B: Cy5). When this primer gets incorporated, it is incorporated into double stranded product and not accessible to hybridization.

After PCR, end-point reading is performed (usually at room temperature). A “quencher oligonucleotide” with 3′ quencher that has sequence complementarity to at least part of the tag sequence will hybridize to unincorporated fluorescent primers and quench their fluorescent signal (The 3′ quencher automatically blocks the oligos from serving as PCR primers). Fluorescent primers incorporated into PCR product will emit a signal.

### Required Oligos Per Assay:

Reverse primer.

2 Allele-specific forward primers A and B, both with a different 20-30 bp tag.

Tag-specific Fluorescent “F2” primers. Different assays may use the same tags and thus F2 primers.

F2 complementary quencher oligos. Usually 2, but the tags could be designed to have enough sequence homology that the same quencher oligo works for both tags.

### Variations

The fluorescent reading can also be carried out at higher temperatures, depending on the length and Tm of the quencher oligo. With longer quencher oligos, real-time PCR detection and melting analysis may be possible. (Real-time PCR detection has already been performed with this setup for digital PCR by the inventor, but not in allele specific fashion).

There are many possibilities to design quencher oligos: length, mismatches to tag, modified bases etc.

Reverse primer also carries a tag (or is longer, has higher Tm than target specific part of F primers) such that annealing temperature of PCR can be raised after 2 or more cycles (after 1 if R is just longer) to prevent new annealing of F primers to the target sequence. F primers will only anneal to product synthesized in earlier cycles that includes the tag sequence.

Both F and R primers are allele-specific (overlap of 1-3 nt) and carry a tag.

Allele-specific F primer at low concentration. Asymmetric reaction setup.

Each tagged primer can carry the fluorescent dye directly, which however increases cost for multiple assays.

Fluorescent primer detection system can be replaced by using universal target specific dual labeled probes that are complementary to the tags and are degraded by 5′ exonuclease (TaqMan like).

The performances of the dual labeled TaqMan probes, especially for end point, may be enhanced by a quencher oligo.

TaqMan probes may be replaced by a pair of (at least in part) complementary oligos where one carries a fluorescent dye (e.g. 5′) and the other a quencher (3′). One or both oligos may be cleaved through 5′ exonuclease (more precisely 5′ FLAP endonuclease) during PCR amplification.

Allele-Specific PCR with Tanned Primers, Labeled Tan-Specific Primers, with Double-Stranded DNA-Binding Dye Quenching

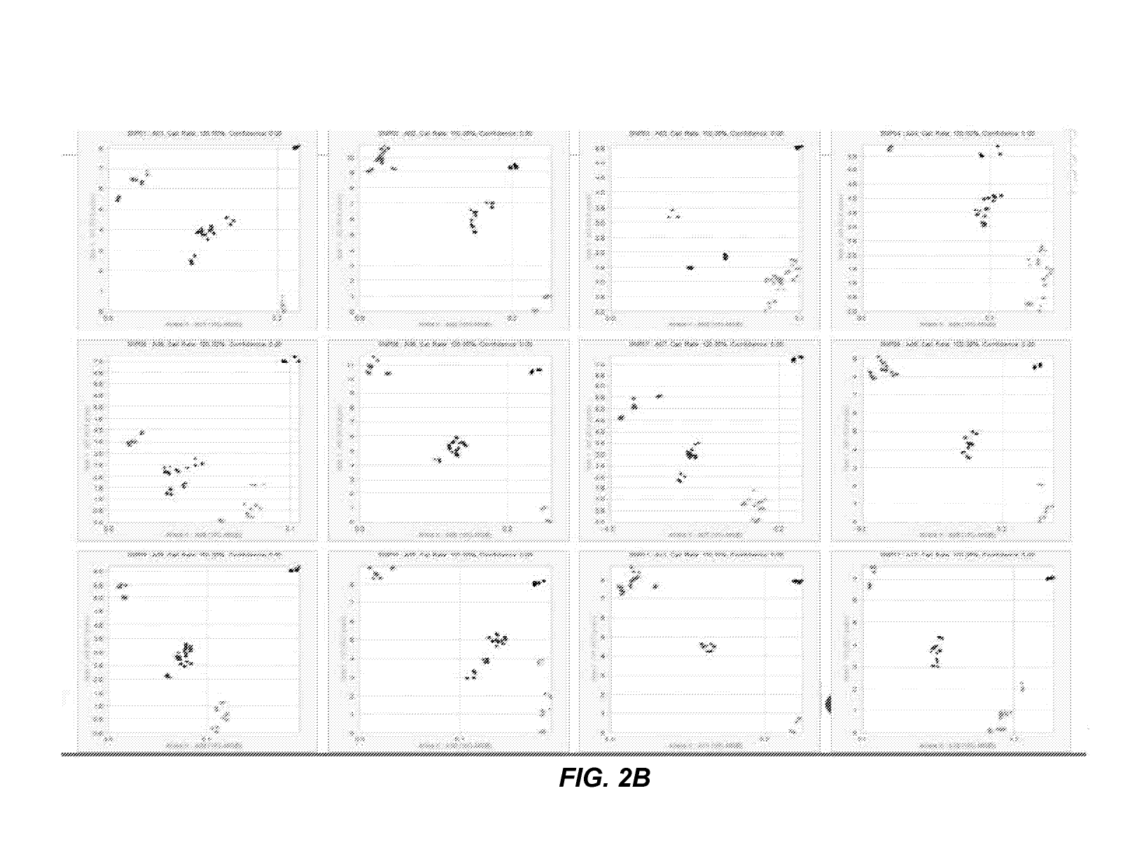

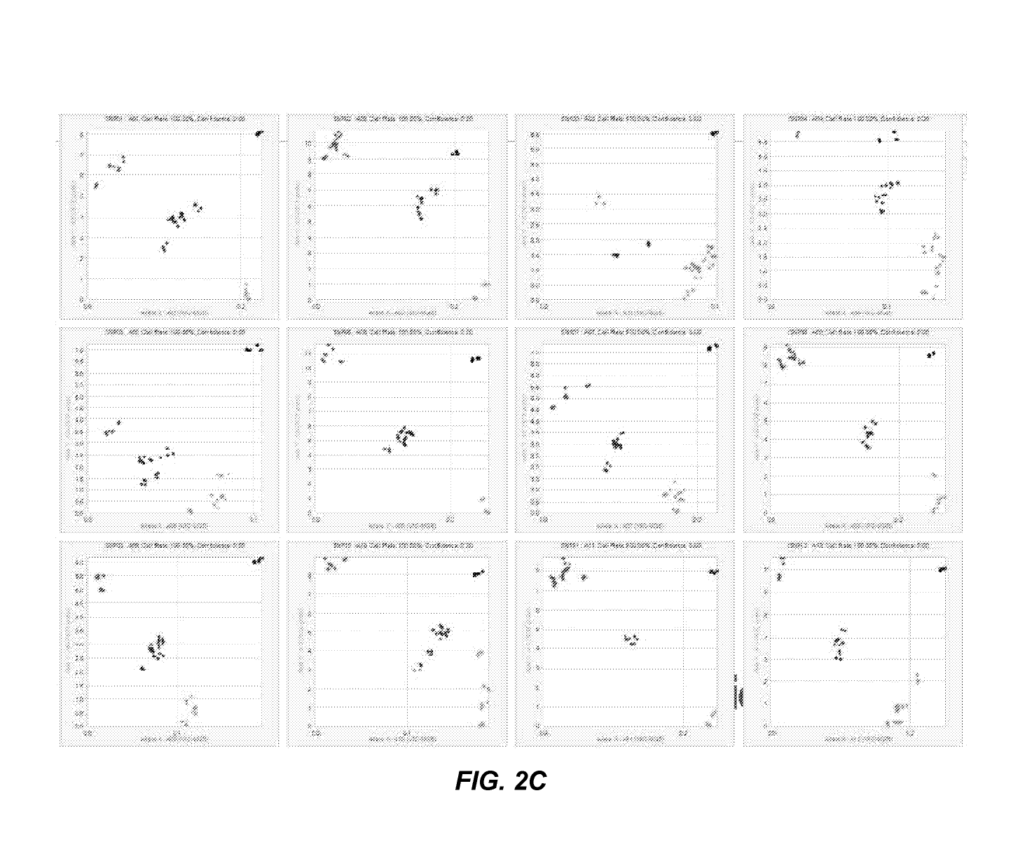

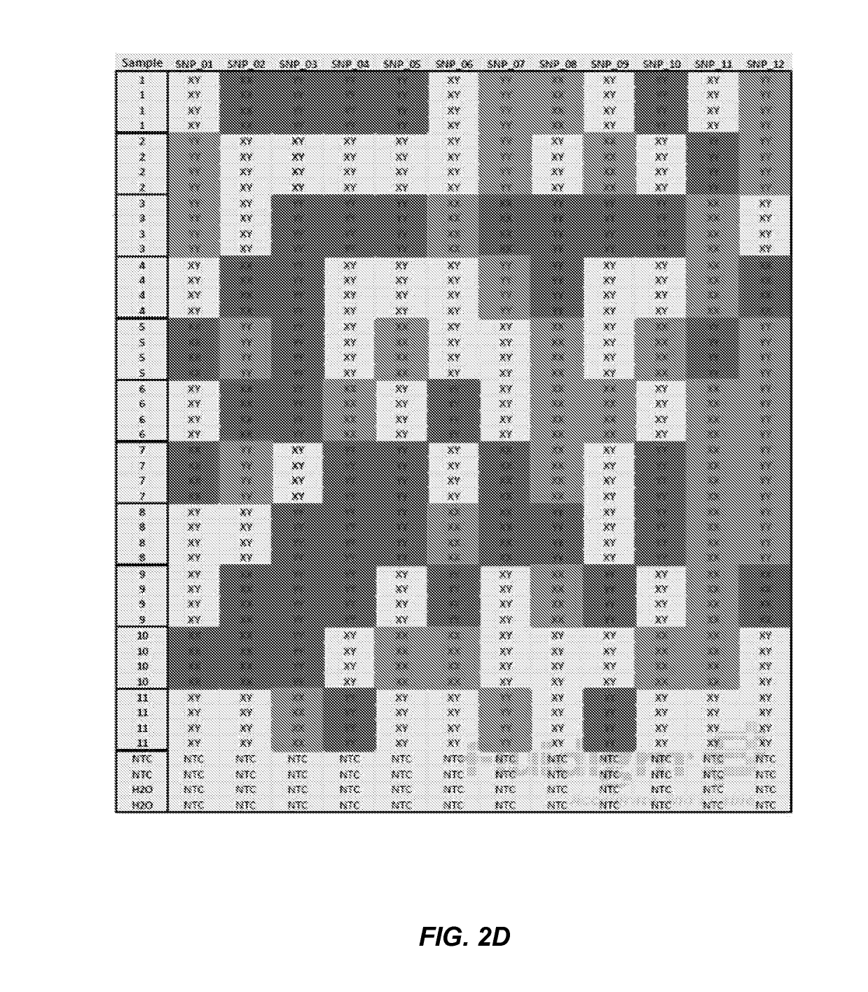

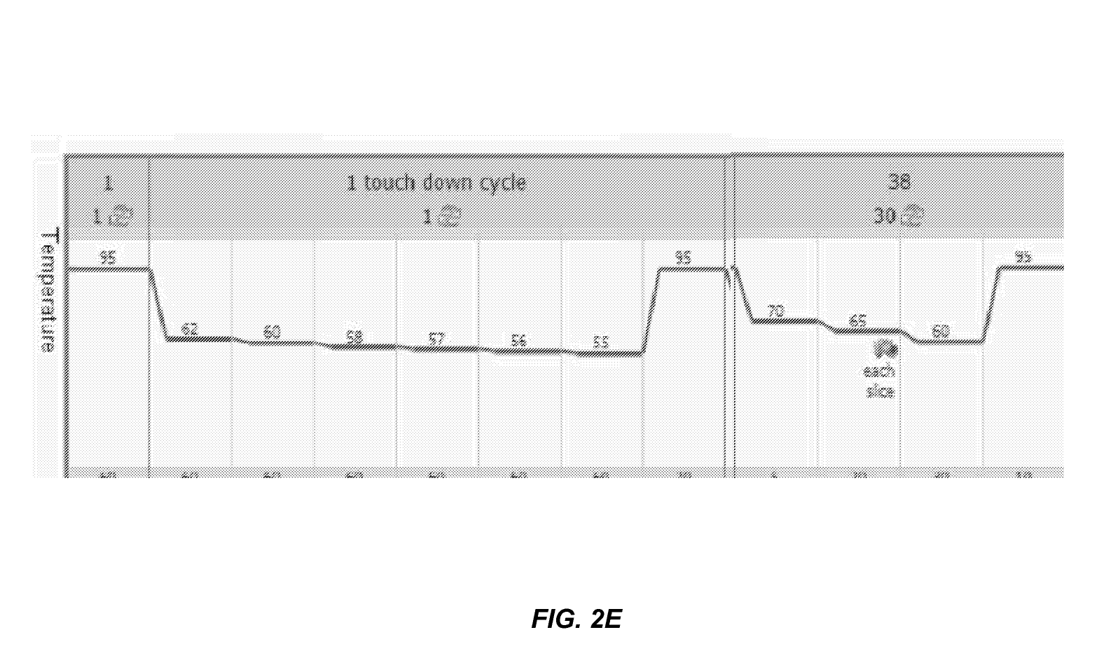

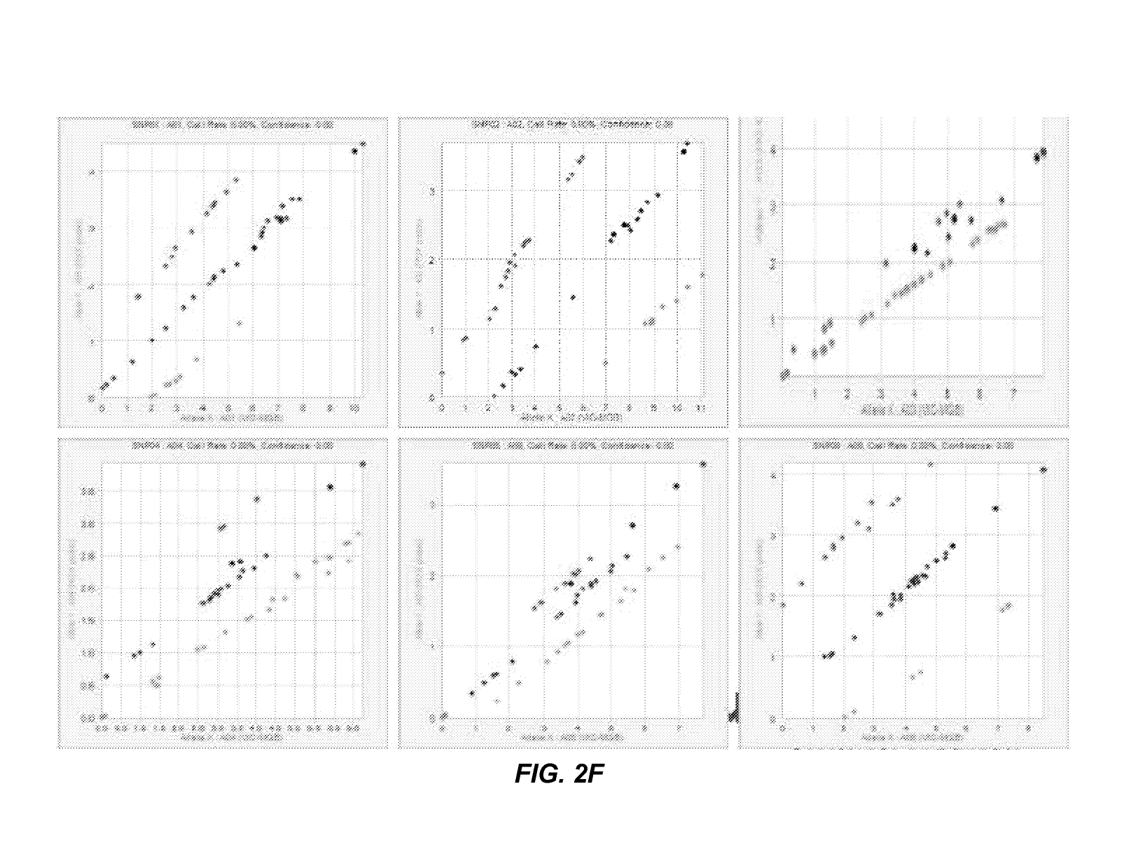

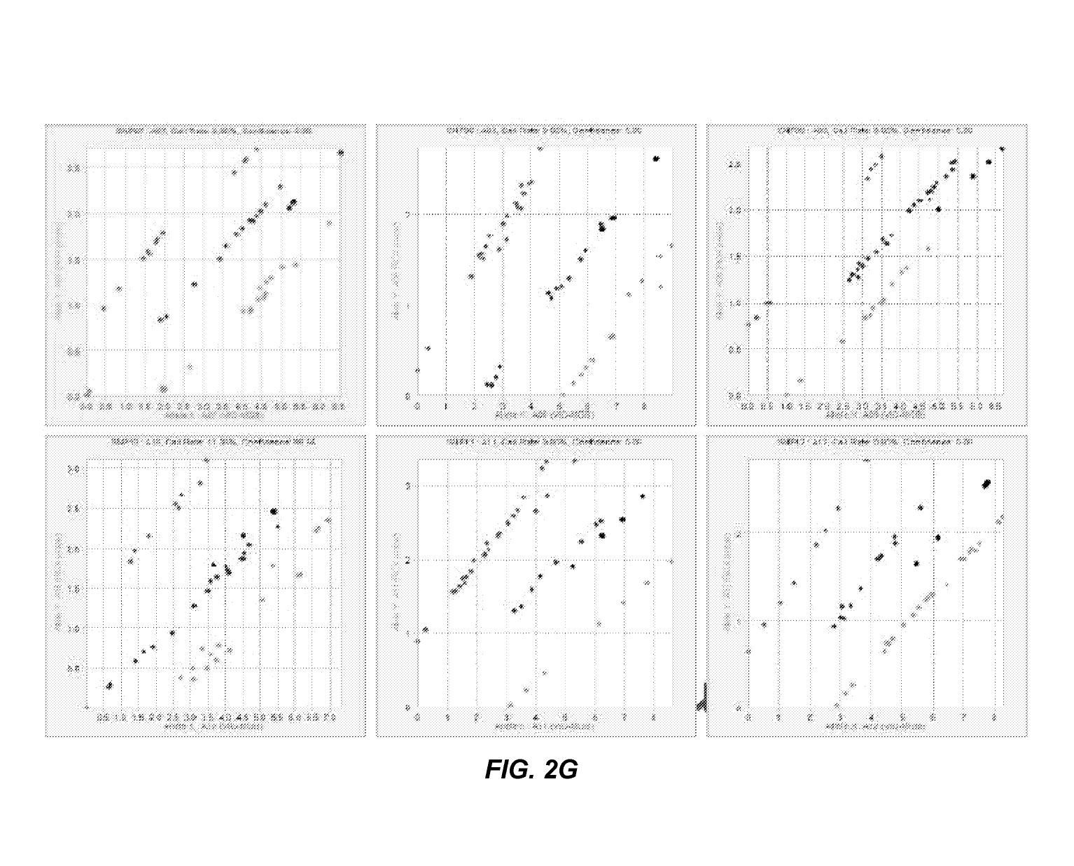

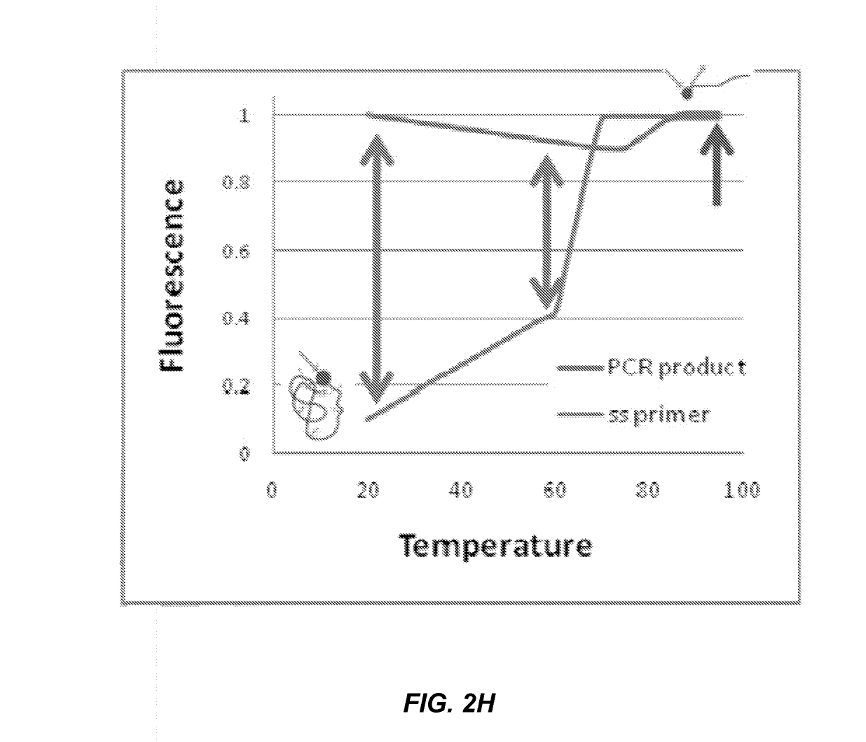

To detect the allele for a particular locus that is present in a sample, the sample nucleic acids were subjected to allele-specific PCR using two forward allele-specific primers that included 5′ nucleotide tags having different nucleotide sequences and a common reverse primer. See FIG. 2A. The amplification reaction included two tag-specific primers, each with a different fluorescent label at the 5′ end and a double-stranded DNA-binding dye. Single-stranded primers give a fluorescent signal. Eva Green binds to the PCR product and quenches signal.

Experimental

12 SNPs of expanded genotype performance test.

11 DNAs of expanded genotype performance test, pre-amplified.

Designs according to Tm shift Advanced Development Protocol (see also U.S. Patent Publication No. 20060172324, published Aug. 3, 2006, which is incorporated by reference herein in its entirety and specifically for this disclosure).

Protocol similar to Tm shift Advanced Development Protocol.

SNPs 1 to 12 (FIG. 2B):

“EP” read after 25 cycles

Inverted graphs

SNPs 1 to 12 (FIG. 2C):

Signal is actually negative

All Calls were Correct (FIG. 2D)

Manual Calls

11 samples

4 replicates

12 assays

=528 of 528 correct

Also worked as well with different R concentration.

Assay

Pre-amplification for 14 cycles (10 ng DNA).

PCR in M48 GT:
2 Allele-specific primers with 2 universal tags (i.e. same 2 tags for each SNP): 100 nM. TA<54° C.
   1 common Reverse primer: 250 nM (100 nM equal). TA>60° C.
Two fluorescent, Forward primers specific to universal tags (FtagA: CAL

Fluor Orange; FtagB: CAL Fluor Red): 100 nM each. TA=55° C.
Fluorescent dye: Eva Green 1×.
GTXpress PCR mix (AB).
Sample loading reagent SG.
Reference during PCR: Eva Green to detect amplification

Post run insertion of other reference (Vic at cycle 1).

PCR (FIG. 2E)

1st cycle: 6 minutes touchdown annealing between 62 and 55° C.

Promote annealing of correct SNPs primer

Introduce tag (for correct SNP this increases Tm>65° C.)

30 cycles: 1 minute touchdown annealing between 70 and 60° C.

Fam as reference: Eva Green signal as control (M48 GT can only read 2 probes and a reference)

Used reading at cycle 25 for analysis and exchange reference picture.

Exchange Reference

Eva Green signal increases with amplification and is not ideal as reference.

Use data from Vic cycle 1 as reference.

Use data from Vic and Rox of cycle 25.

Analyze as EP (endpoint) run.

Eva Green as Reference

SNPs 1 to 6 (FIG. 2F): lines instead of clusters, but can be called (but NTC), as shown below.

SNPs 7 to 12 (FIG. 2G).

Conclusion

12 of 12 SNPs worked.

Cheap chemistry using universal fluorescent primers.

Denaturation removes quenching effect.

Currently employs preAmp.

Manual calling.

Exposure settings suboptimal (Tcalibration, negative PCR signal).

SNP-Methods

EP (endpoint) at RT (room temperature)

Melt analysis

Eva->LCG->Fam as second color

Other DNA binding dyes (& ssDNA binding)

Rox as reference (in mix)->Quasar for allele B

Reference at 95° C.

Anti-Quencher oligo to fluorescent primers (worked before)

iFRET (excite Eva->read CaR fluorescence)

Optimize PCR protocol

Cheaper preAmp (now $0.20 per sample)

Temperature Dependence of Signal (FIG. 2H)

Real-time PCR at 60° C. possible

EP (endpoint) read very strong signal/noise.

At 95° C.: positive and negative reactions have same signal, (primer and PCR product are single stranded)

### Example 3

### Use of Target-Complementary Oligo and a Tag-Specific Primer to Generate Target-Specific Tagged Primers

### Problem:

Problem 1: In the access array protocol for producing PCR products with Sequencing tags and sample barcode, we currently use a four primer protocol: 2 inner primers are target specific and have a 5′ tag. The outer primers are specific to the tags of the inner primers, may carry a barcode sequence that allows identification of the sample in sample mixtures, and have the sequencing adapter sequence on their 5′-end. This protocol may cause uneven incorporation of the sequencing tag between PCR assays. This problem is increased when one tries to multiplex the PCR reactions for tagging (in the access array).

Problem 2: Find a cheap genotyping chemistry

### Solution:

Solution 1: The inner tagged primer is replaced by its complement. In a first phase, some of the outer primer will dimerize with this complement and be extended by polymerase into a target specific primer that carries the full set of tag information. These primers will be able to prime of the target sequence and form a full length product. Once a product has the full tagging incorporated on both side, it will further amplify with the outer primers.

Solution 2: Two (labeled) tag-specific primers are used for the detection of individual alleles. These will have different sequences. 2 target complementary oligos (one per allele) with a 3′-tag complementary to one of the 2 fluorescent primers oligo is used to generate functional fluorescent allele specific primers, in the reaction. Detection of allele specific product can be performed by several tag-specific detection methods, e.g. using a quencher oligo that binds to the labeled tag if it is not in a ds PCR product. Or use of tag-specific dual labeled probes. The same detection system can be used for any pair of target sequences.

### Scope:

The possibility of generating functional long primers by combining two oligos as described above may have multiple applications. It will be useful in instances where tag sequences are appended to target specific primers. Especially in instance where one target is amplifies to carry multiple different tags, e.g. one tag per sample.

For the SNP method, the same detection system can be used for any pair of target sequences. One optimized detection system will fit all.

### Possible Variations:

The general protocol for generating primers is relatively simple.

In general, the concentration of the outer tag primer will be greater than the concentration of the inner complementary oligo. This assures that there is free functional long primer present, not blocked by hybridizing to the complementary oligo. The complementary oligo may be blocked from extension or can be extended using the outer tag as target, whichever is better for the protocol.

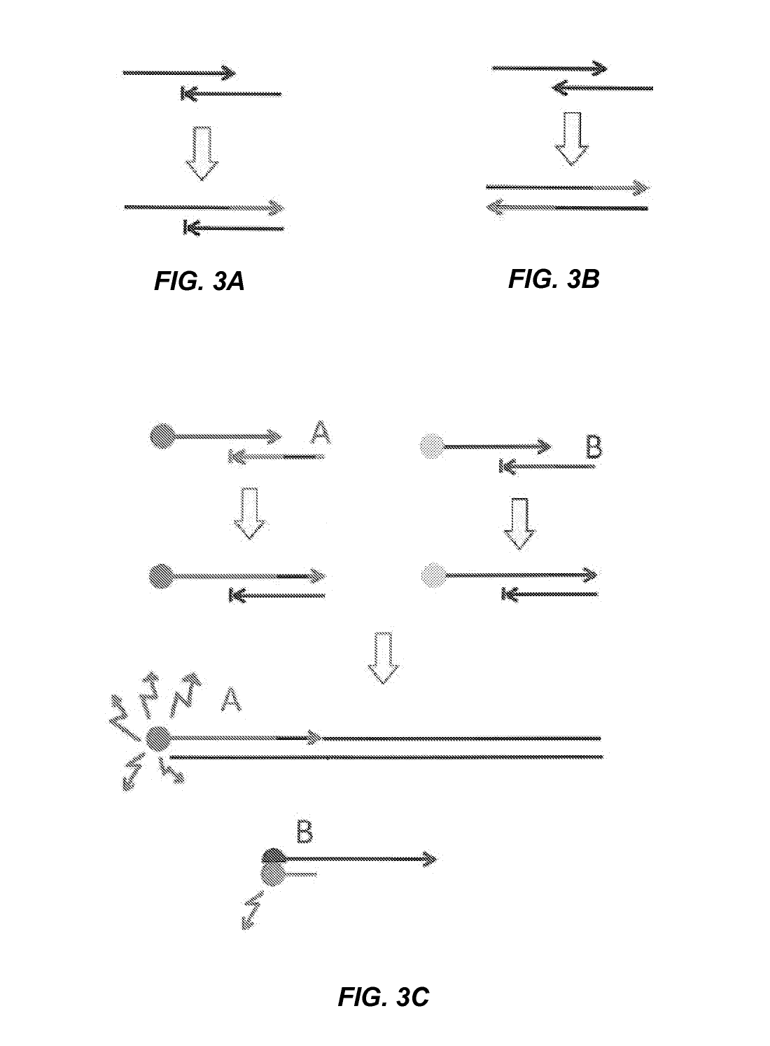

Variations of the methods described in this Example are shown in FIG. 3. In FIG. 3A, the complementary oligonucleotide is blocked; only the outer forward primer is extended into the full-length primer. In FIG. 3B, the complementary oligonucleotide is not blocked; forward primer and complimentary oligonucleotide are extended into full-length primer and complement. In FIG. 3C, allele-specific long forward primers are generated from extending fluorescent tag primers hybridized to their respective allele's complimentary oligonucleotides. In this illustration, the sample is homozygous for allele A. The fluorescent primers of allele A get incorporated into PCR product and generate fluorescence, while allele B's primers hybridize to a quencher oligonucleotide and generate no fluorescent signal.

### Example 4

### Ligation Assays for Detecting Fetal Aneuploidy

The ability to use Digital Array™ IFCs to detect fetal aneuploidy can be limited by the amount of fetal DNA isolated from maternal plasma. One way to address this problem is to use multiple assays for each chromosome being counted. 50 UPL assays for chromosome 18 and 50 UPL assays for chromosome 21 were designed. The chr18 assays all use UPL probe 019 and the chr21 assays all use UPL probe 020. From these, 10 chr18 assays and 10 chr21 assays were selected. These are run as a multiplex so there is a mixture of 40 different primers and 2 probes in the test. Eventually, it might desirable to run 100 assays per chromosome, or even more, in order to improve sensitivity for a diagnostic test.

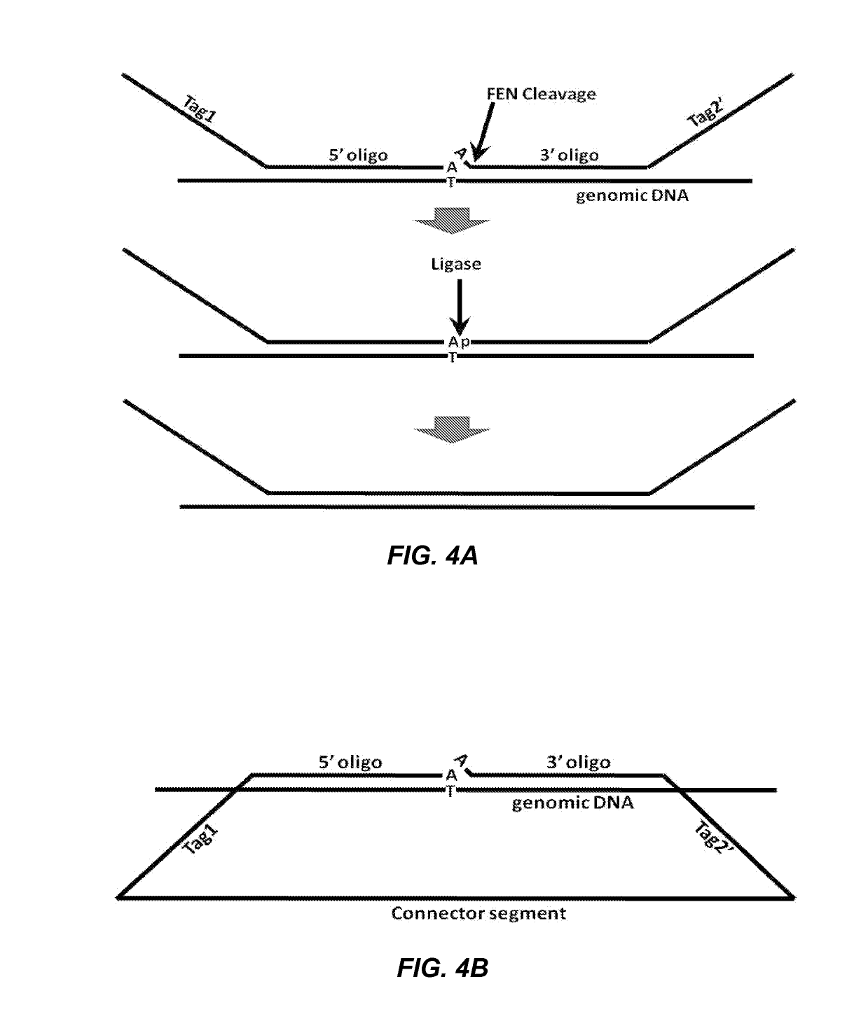

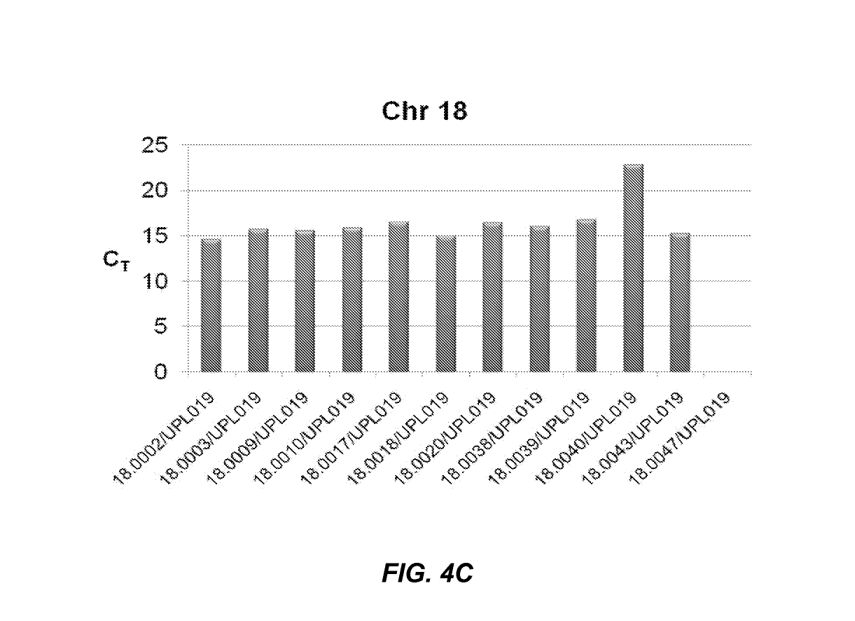

An alternative multiplexing scheme is to use ligation to create DNA templates so that all assays from the same chromosome use the same pair of PCR primers. This scheme is illustrated in FIG. 4A.

This scheme has a number of attributes: FEN is flap endonuclease. FEN cleaves most readily when the displaced nucleotide is complementary to the nucleotide in the template (genomic DNA). If the displaced nucleotide is not complementary, the rate of FEN cleavage is typically 100 times slower. Thus, FEN cleavage essentially occurs only when the 5′ oligo and 3′ oligo are hybridized next to each other and properly aligned so that the complementary nucleotide in the 3′ oligo is displaced.

FEN cleavage generates a 5′ phosphate on the 3′ oligo. Ligase requires a 5′ phosphate in order to seal DNA nicks. In the absence of FEN cleavage (which requires proper hybridization and alignment), there are no oligos present with a 5′ phosphate and thus no oligos can be ligated together. This drastically reduces non-specific background ligation compared to other ligase assays.

Ligation occurs most readily when the 3′ nucleotide of the 5′ oligo is complementary to the template nucleotide. Thus, ligation essentially occurs only when the 5′ and 3′ oligos are properly hybridized and aligned such that the 3′ nucleotide of the 5′ oligo is base-paired to the template strand and next to a 5′ phosphate on the 3′ oligo.

If ligation occurs, then a ligation product is formed that can be PCR amplified using primers Tag1 and Tag2. (Tag2′ refers to the reverse complement of the Tag2 sequence.) The same Tag1 and Tag2 sequences will be used for all ligase assays for a given chromosome.

The 5′ nuclease domain of Taq DNA polymerase is a FEN. Thus, one way to generate FEN activity is to use Taq DNA polymerase in the absence of dNTPs. With no dNTPs present, Taq DNA polymerase has no polymerase activity.

After ligation, the ligation oligos need to be removed because they will interfere with subsequent PCR steps. There are a number of methods for removing the unligated ligation oligos:

(1) Chromatography or ultrafiltration. Unligated oligos can be separated from ligation products by passing over a Sephadex column or using ultrafiltration devices such as the Sartorus Vivaspin 500 ultrafiltration spin columns. This separation is enhanced if the ligation product remains hybridized to the genomic DNA. Thus, this method for oligo clean-up works best using only a single round of ligation.

(2) Blocked oligos. The 5′ end of the 5′ oligo and the 3′ end of the 3′ oligo can be blocked to exonuclease digestion by adding one or two phosphorothioate or 2′-O-Methyl nucleotides. After ligation, the reaction is treated with exonuclease. Ligation products have both ends blocked and thus are not digested by the exonuclease. Unligated oligos have one free end and thus are digested.

(3) Circular ligation product. The 5′ oligo and 3′ oligo can be joined using a connector segment so there is only one ligation oligo per assays. See FIG. 4B.

Upon ligation, a circular ligation product is formed which is resistant to exonuclease digestion. So, again the ligation reaction can be treated with exonuclease to digest any unligated oligos.

For either method using exonuclease digestion, multiple rounds of ligation can be performed if one is using a thermostable ligase. This results in linear amplification of the ligation products.

Starting with UPL assays, ligation assays were designed for 12 loci on chromosome 18 and 12 loci on chromosome 21. Assays were designed so that ligation occurs within the UPL probe sequence. Also, the 5′ and 3′ oligos were designed to have a 2-nt overlap. Using IDT OligoAnalyzer 3.1 with default salt and oligo concentrations, 5′ and 3′ oligos were designed to have a Tₘ in the range 58° C. to 60° C. Strand selection for the oligos was made by first avoiding oligos with 4 or more G's in a row, then by selecting the strand that has the higher C-to-G ratio.

The probe and tag sequences used in this design are given in Table 1.

TABLE 1

| | | Tm |
|---|---|---|
| Chrosmosome 18 | | |
| UPL019 | ctccagcc | |
| UPL019′ | ggctggag | |
| Tz_18F | TCAATTCCAGGTGTGCGAAA | 54.9 |
| Tz_18R | TGGACGAGCAACAGCACTATAAA | 56.4 |
| Tz_18R′ | TTTATAGTGCTGTTGCTCGTCCA | |
| Chrosmosome 21 | | |
| UPL20 | ccagccag | |
| UPL20 | ctggctgg | |
| T020 | TGCAACGAGTTAGTGGAACAGAAT | 56.6 |
| T021 | ACAGCACAACTCGCAATTGAA | 55.6 |
| T021′ | TTCAATTGCGAGTTGTGCTGT | |

For chromosome 18 assays, the Tz_{—}18F sequence was added to the 5′ end of the 5′ oligo and the Tz_{—}18R′ sequence was added to the 3′ end of the 3′ oligo. For chromosome 21 assays, the T020 sequence was added to the 5′ end of the 5′ oligo and the T021′ sequence was added to the 3′ end of the 3′ oligo.

For each loci, two PCR primers were also designed. This was done by moving away from the UPL probe sequence until an A (or sometimes T) was encountered, then picking a primer with an IDT Tₘ in the range 55° C. to 57° C. These primers have some tag sequence at the 5′ end and locus-specific sequence at the 3′ end. These primers will be used to evaluate the yield of ligation product for each locus-specific assay.

The oligos that were designed for the twelve chr18 assays and twelve chr21 assays are given in Table 2.

TABLE 2

| Well | Name | Sequence | Length | Tm |
|---|---|---|---|---|
| A01 | 18_0002_Tz18F_L51 | TCAATTCCAGGTGTGCGAAAGCAGATCCCCCTGCTCCA | 38 | |
| A02 | 18_0002_L31_Tz18R | CAGCCTATTTTTCAGTTGGCATGGTGTTTATAGTGCTGTTGCTCGTCCA | 49 | |
| A03 | 18_0002_PF1 | CAGGTGTGCGAAAGCAGA | 18 | 55.6 |
| A04 | 18_0002_PR1 | CACTATAAACACCATGCCAACTGAA | 25 | 55.6 |
| A05 | 18_0003_Tz18F_L51 | TCAATTCCAGGTGTGCGAAACTCGCCATCTCCGGCTG | 37 | |
| A06 | 18_0003_L31_Tz18R | TGGAGAGTTAATTAATCCCATCTCCCACTTAACTTTATAGTGCTGTTGCTCGTCCA | 56 | |
| A07 | 18_0003_PF1 | GTGTGCGAAACTCGCCA | 17 | 56.2 |
| A08 | 18_0003_PR1 | ACTATAAAGTTAAGTGGGAGATGGGATTAA | 30 | 55.6 |
| A09 | 18_0009_Tz18F_L51 | TCAATTCCAGGTGTGCGAAACACAATCCACCCCAGATTTTTATCTCCA | 48 | |
| A10 | 18_0009_L31_Tz18R | CAGCCTCTGTTCTCTTTACAGCTTCTGTAGTTTATAGTGCTGTTGCTCGTCCA | 53 | |
| A11 | 18_0009_PF1 | GAAACACAATCCACCCCAGA | 20 | 55.0 |
| A12 | 18_0009_PR1 | GCACTATAAACTACAGAAGCTGTAAAGAGAA | 31 | 56.5 |
| B01 | 18_0010_Tz18F_L51 | TCAATTCCAGGTGTGCGAAAGCAAGATGCTGGCTTACCTCCA | 42 | |
| B02 | 18_0010_L31_Tz18R | CAGCCTTATCAGCCTTGATCCTAGCTTTATAGTGCTGTTGCTCGTCCA | 48 | |
| B03 | 18_0010_PF1 | CGAAAGCAAGATGCTGGCTTA | 21 | 55.8 |
| B04 | 18_0010_PR1 | CACTATAAAGCTAGGATCAAGGCTGATAA | 29 | 56.1 |
| B05 | 18_0017_Tz18F_L51 | TCAATTCCAGGTGTGCGAAAGTCTCTCTGACAGTCTCACTACTCCA | 46 | |
| B06 | 18_0017_L31_Tz18R | CAGCCTTTGAGCTGCCTAAGGTTTATAGTGCTGTTGCTCGTCCA | 44 | |
| B07 | 18_0017_PF1 | GAAAGTCTCTCTGACAGTCTCACTA | 25 | 55.2 |
| B08 | 18_0017_PR1 | GCACTATAAACCTTAGGCAGCTCAA | 25 | 57.0 |
| B09 | 18_0018_Tz18F_L51 | TCAATTCCAGGTGTGCGAAACCTCTTTCGTGGGCTCTCCA | 40 | |
| B10 | 18_0018_L31_Tz18R | CAGCCGGGAAATCCATCAACAGTTTATAGTGCTGTTGCTCGTCCA | 45 | |
| B11 | 18_0018_PF1 | GTGTGCGAAACCTCTTTCGT | 20 | 55.8 |
| B12 | 18_0018_PR1 | CAACAGCACTATAAACTGTTGATGGA | 26 | 55.4 |
| C01 | 18_0020_Tz18F_L51 | TCAATTCCAGGTGTGCGAAAGCCAAGTCCTGCATATTTATCCTCCA | 46 | |
| C02 | 18_0020_L31_Tz18R | CAGCCCACTGGTACCCTGTTTATAGTGCTGTTGCTCGTCCA | 41 | |
| C03 | 18_0020_PF1 | CGAAAGCCAAGTCCTGCATA | 20 | 55.3 |
| C04 | 18_0020_PR1 | AACAGCACTATAAACAGGGTACCA | 24 | 55.9 |
| C05 | 18_0038_Tz18F_L51 | TCAATTCCAGGTGTGCGAAACCCCAAGAATCGGGACTCCA | 40 | |
| C06 | 18_0038_L31_Tz18R | CAGCCTGGTGATTCATCATGCCTTTTATAGTGCTGTTGCTCGTCCA | 46 | |
| C07 | 18_0038_PF1 | CGAAACCCCAAGAATCGGGA | 20 | 57.4 |
| C08 | 18_0038_PR1 | CACTATAAAAGGCATGATGAATCACCA | 27 | 55.5 |
| C09 | 18_0039_Tz18F_L51 | TCAATTCCAGGTGTGCGAAAGCATGGGAACAGCCTCCA | 38 | |
| C10 | 18_0039_L31_Tz18R | CAGCCTCAGGGACCAGGTTTATAGTGCTGTTGCTCGTCCA | 40 | |
| C11 | 18_0039_PF1 | GTGCGAAAGCATGGGAACA | 19 | 56.3 |
| C12 | 18_0039_PR1 | GCACTATAAACCTGGTCCCTGA | 22 | 56.4 |
| D01 | 18_0040_Tz18F_L51 | TCAATTCCAGGTGTGCGAAACAGGAGGGCACACCTCCA | 38 | |
| D02 | 18_0040_L31_Tz18R | CAGCCCCACAGAGCAGGTTTATAGTGCTGTTGCTCGTCCA | 40 | |
| D03 | 18_0040_PF1 | CGAAACAGGAGGGCACA | 17 | 55.1 |
| D04 | 18_0040_PR1 | AACAGCACTATAAACCTGCTCTGT | 24 | 56.3 |
| D05 | 18_0043_Tz18F_L51 | TCAATTCCAGGTGTGCGAAAGCAGGCTACTGTCCATTCTCCA | 42 | |
| D06 | 18_0043_L31_Tz18R | CAGCCCTGGATACAGAGCCACTTTATAGTGCTGTTGCTCGTCCA | 44 | |
| D07 | 18_0043_PF1 | CGAAAGCAGGCTACTGTCCA | 20 | 57.2 |
| D08 | 18_0043_PR1 | GCACTATAAAGTGGCTCTGTATCCA | 25 | 56.4 |
| D09 | 18_0047_Tz18F_L51 | TCAATTCCAGGTGTGCGAAACAATTTTGCAGTCCTTGACATCTCTCCA | 48 | |
| D10 | 18_0047_L31_Tz18R | CAGCCCAGGCCTCAGGTTTATAGTGCTGTTGCTCGTCCA | 39 | |
| D11 | 18_0047_PF1 | CGAAACAATTTTGCAGTCCTTGAA | 24 | 56.6 |
| D12 | 18_0047_PR1 | AGCACTATAAACCTGAGGCCT | 21 | 55.7 |
| E01 | 21_0001_T020_L51 | TGCAACGAGTTAGTGGAACAGAATCACCAACCACATTCAAAGCCAGC | 47 | |
| E02 | 21_0001_L31_T021 | GCCAGAGGCCATTGTCCAAGATTCAATTGCGAGTTGTGCTGT | 42 | |
| E03 | 21_0001_PF1 | GAACAGAATCACCAACCACATTCAA | 25 | 55.9 |
| E04 | 21_0001_PR1 | CAACTCGCAATTGAATCTTGGACAA | 25 | 56.3 |
| E05 | 21_0005_T020_L51 | TGCAACGAGTTAGTGGAACAGAATCCACCCGTGCCAGC | 38 | |
| E06 | 21_0005_L31_T021 | GCCAGTTTCCATATCAGCCAGGTTCAATTGCGAGTTGTGCTGT | 43 | |
| E07 | 21_0005_PF1 | TGGAACAGAATCCACCCGT | 19 | 56.3 |
| E08 | 21_0005_PR1 | CAATTGAACCTGGCTGATATGGAA | 24 | 55.4 |
| E09 | 21_0008_T020_L51 | TGCAACGAGTTAGTGGAACAGAATCCACCATCTCTTCCACTCTGGC | 46 | |
| E10 | 21_0008_L31_T021 | GCTGGCTTCCCTTCTTCCTTTCTGTTCAATTGCGAGTTGTGCTGT | 45 | |
| E11 | 21_0008_PF1 | AACAGAATCCACCATCTCTTCCA | 23 | 55.9 |
| E12 | 21_0008_PR1 | CAATTGAACAGAAAGGAAGAAGGGAA | 26 | 55.6 |
| F01 | 21_0013_T020_L51 | TGCAACGAGTTAGTGGAACAGAATGGCCGGACTCCCAGC | 39 | |
| F02 | 21_0013_L31_T021 | GCCAGAGCCAATAACCAGCACTTCAATTGCGAGTTGTGCTGT | 42 | |
| F03 | 21_0013_PF1 | GAACAGAATGGCCGGACT | 18 | 54.9 |
| F04 | 21_0013_PR1 | CAATTGAAGTGCTGGTTATTGGCT | 24 | 56.2 |
| F05 | 21_0015_T020_L51 | TGCAACGAGTTAGTGGAACAGAATCTTTCTGTTATCATCTCAGCCTTCCAGC | 52 | |
| F06 | 21_0015_L31_T021 | GCCAGAAAGAAGGAAGCGTCCATTTCAATTGCGAGTTGTGCTGT | 44 | |
| F07 | 21_0015_PF1 | TGGAACAGAATCTTTCTGTTATCATCTCA | 29 | 56.0 |
| F08 | 21_0015_PR1 | CAATTGAAATGGACGCTTCCTTCTT | 25 | 56.2 |
| F09 | 21_0019_T020_L51 | TGCAACGAGTTAGTGGAACAGAATCTGCCTTCTGCTCCCAGC | 42 | |
| F10 | 21_0019_L31_T021 | GCCAGAGTGAGAGCGGAGTTCAATTGCGAGTTGTGCTGT | 39 | |
| F11 | 21_0019_PF1 | GAACAGAATCTGCCTTCTGCT | 21 | 55.1 |
| F12 | 21_0019_PR1 | GCAATTGAACTCCGCTCTCA | 20 | 55.5 |
| G01 | 21_0021_T020_L51 | TGCAACGAGTTAGTGGAACAGAATGCTCAGAACACCTAGAGCTGGC | 46 | |
| G02 | 21_0021_L31_T021 | GCTGGGCCACGTCCCTTCAATTGCGAGTTGTGCTGT | 36 | |
| G03 | 21_0021_PF1 | GGAACAGAATGCTCAGAACACCTA | 24 | 56.7 |
| G04 | 21_0021_PR1 | CTCGCAATTGAAGGGACGT | 19 | 55.4 |
| G05 | 21_0032_T020_L51 | TGCAACGAGTTAGTGGAACAGAATACTTGCAGATCCAGTTCCCAGC | 46 | |
| G06 | 21_0032_L31_T021 | GCCAGCTGGAATCAGTTCTGCTTTCAATTGCGAGTTGTGCTGT | 43 | |
| G07 | 21_0032_PF1 | TGGAACAGAATACTTGCAGATCCA | 24 | 56.1 |
| G08 | 21_0032_PR1 | GCAATTGAAAGCAGAACTGATTCCA | 25 | 56.4 |
| G09 | 21_0036_T020_L51 | TGCAACGAGTTAGTGGAACAGAATCTCCTGCTTGTCTTTCAGAACCAGC | 49 | |
| G10 | 21_0036_L31_T021 | GCCAGGTGTAGACCTGGGACTTCAATTGCGAGTTGTGCTGT | 41 | |
| G11 | 21_0036_PF1 | AGAATCTCCTGCTTGTCTTTCAGAA | 25 | 56.1 |
| G12 | 21_0036_PR1 | GCAATTGAAGTCCCAGGTCTACA | 23 | 57.0 |
| H01 | 21_0041_T020_L51 | TGCAACGAGTTAGTGGAACAGAATTCACCCCATAGCCACCAGC | 43 | |
| H02 | 21_0041_L31_T021 | GCCAGCATTCAGCACAGCAGTTCAATTGCGAGTTGTGCTGT | 41 | |
| H03 | 21_0041_PF1 | ACAGAATTCACCCCATAGCCA | 21 | 56.2 |
| H04 | 21_0041_PR1 | GCAATTGAACTGCTGTGCTGAA | 22 | 56.7 |
| H05 | 21_0046_T020_L51 | TGCAACGAGTTAGTGGAACAGAATCACAAGGTCTGGTGCTGGC | 43 | |
| H06 | 21_0046_L31_T021 | GCTGGCTTCACTTCCTTTGCACTTCAATTGCGAGTTGTGCTGT | 43 | |
| H07 | 21_0046_PF1 | GGAACAGAATCACAAGGTCTGGT | 23 | 56.9 |
| H08 | 21_0046_PR1 | GCAATTGAAGTGCAAAGGAAGTGAA | 25 | 56.7 |
| H09 | 21_0050_T020_L51 | TGCAACGAGTTAGTGGAACAGAATCTCCCCTAGACAAGTCCTTTATAACCAGC | 53 | |
| H10 | 21_0050_L31_T021 | GCCAGTGCTATGTGCTGCAGTTCAATTGCGAGTTGTGCTGT | 41 | |
| H11 | 21_0050_PF1 | GAATCTCCCCTAGACAAGTCCTTTA | 25 | 55.5 |
| H12 | 21_0050_PR1 | CAATTGAACTGCAGCACATAGCA | 23 | 56.3 |

An experiment was run using the chr18 assays. FEN-ligase reactions contained a mixture of all 5′ oligos and 3′ oligos at a concentration of 25 nM each. Reactions also contained 0.1 unit/4 Ampligase (Epicentre A30201), 0.04 unit/4 Taq DNA polymerase (Epicientre Q8201K), 2 ng/μL denatured human genomic DNA, and 1× Ampligase buffer (Epicentre). A 10⁻⁴ reaction was incubated at 95° C. for 15 sec followed by 10 min at 65° C. After addition of 110 μL TE, the reaction was transferred to a Microcon YM-50 filter unit (Millipore 42409) and centrifuged at 14,000×g for 4 min. An additional 100 μL TE was added to the filter unit and it was centrifuged again at 14,000×g for 4 min. The concentrate was transferred to a sample tube. The filter unit was rinsed by adding 100 μL TE and combining this rinse with the concentrate in the sample tube. Two microliters of this sample was preamplified in a 5-4 reaction containing 1× TagMan® PreAmp Master Mix (Applied Biosystems 4391128), 50 nM Tz_{—}18F, and 50 nM Tz_{—}18R. Thermal cycling was 10 min at 95° C. followed by 18 cycles of 95° C. for 15 sec, 60° C. for 4 min. The reaction was stopped by adding 50 μL TE. This material was evaluated using locus-specific assays containing 1× TagMan® Gene Expression Master Mix (Applied Biosystems 4369016), 1× Gene Expression Sample Loading Reagent (Fluidigm), 200 nM forward primer, 200 nM reverse primer, and 100 nM UPL Probe 019. Thermal cycling was 50° C. for 2 min, 70° for 30 min, 95° C. for 10 min followed by 35 cycles of 96° C. for 1 sec, 70° C. for 5 sec, 60° C. for 1 min. The average C_{T} values for each of the twelve chr18 assays are shown in FIG. 4C.

Two assays showed no (18.0047) or very little (18.0040) ligation product. The other 10 assays, though, show that ligation products were formed at fairly similar amounts. This demonstrates that ligation assays can be performed as a multiplex on human genomic DNA to generate locus-specific ligation products in a fairly uniform manner. Furthermore, these ligation products can be PCR amplified with a common pair of Tag primers.

### Example 5

### Method to Detect Differentially Methylated DNA (i.e., “Methyl SNPs”) Using Tm Enhancing Primers and Fluidgim IFCs

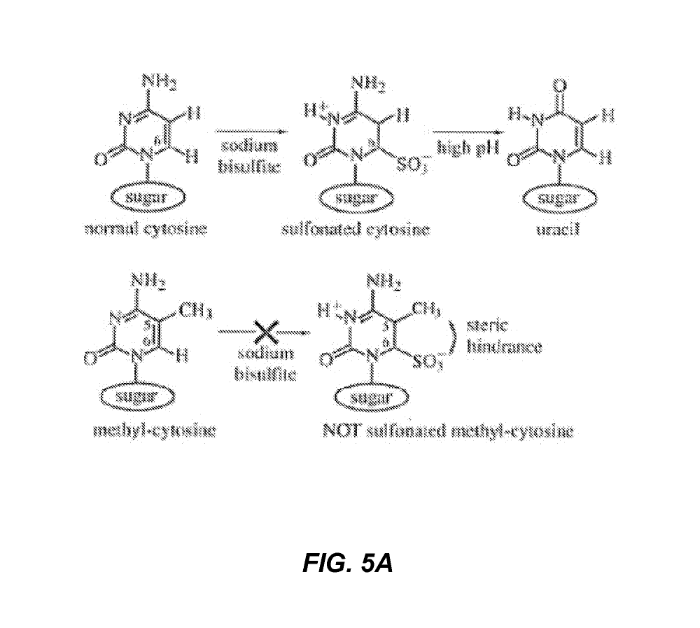

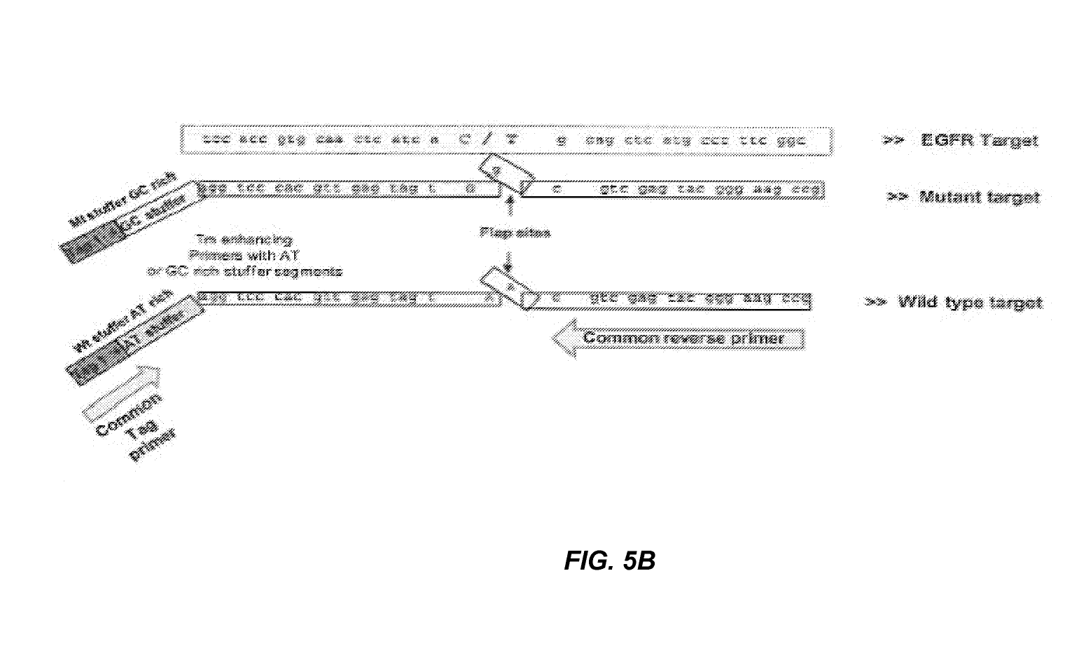

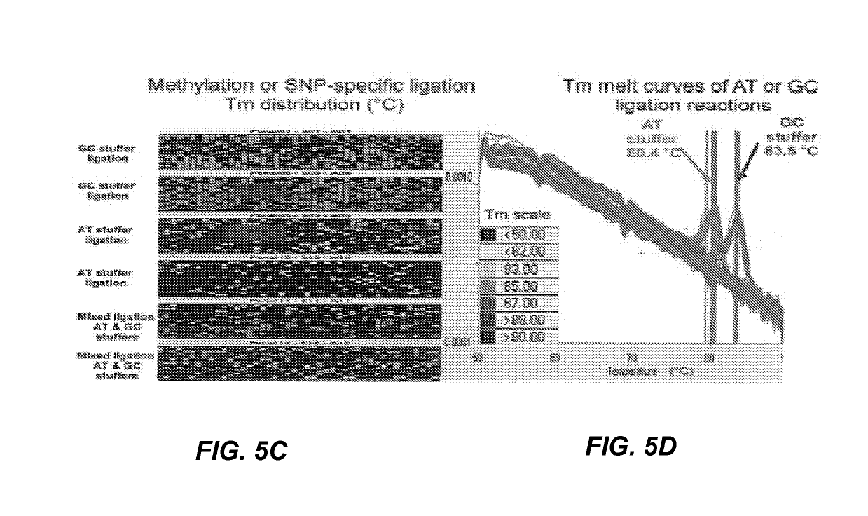

Methylated cytosine can be considered dynamic, single nucleotide polymorphisms of cytosine. Key to discriminating between methylated versus unmethylated cytosines is the use of the widely available chemical, sodium bishuphite (NaHSO₃ is used to clean swimming pools). This simple pre-analytical treatment is described in FIG. 5A. It includes the following steps:

(1) Unmethylated cytosines (C) at high pH are deaminated and converted to uridine (U). These are read as T in the PCR and sequencing reactions.

(2) Methylated cytosines are resistant to bishulphite and continue to be read as C.

(3) Consequently, a comparison of bisulphite treated versus untreated DNA will reveal which cytosines were converted.

### Invention Methodology:

Use of bisulphite treated DNA, Tm enhancing tag primers and oligonucleotide ligation to detect methylated DNA using Fluidigm IFCs

### Generic Bisulphite Protocol:

(1) Treat DNA from either single cells or selected population of cells using commercially available bisulphite conversion kits (Invitrogen, Qiagen etc.).

(2) Employ the highly selective ligase detection assay to hybridize Tm discrimination oligonucleotides to both methylated C (remain as C) and unmethylated (converted to U). See FIG. 5B.

### In a Separate Reaction Vessel Add:

(1) The C-m targeting oligo bearing a long relatively high Tm tag “stuffer” sequence (the oligo 5′ ends may be nuclease resistant).

(2) The U targeting primer contains a shorter, lower Tm tag sequence (the oligo 3′ ends may be nuclease resistant).

(3) A reverse primer bearing an overhanging nucleotide “flap” that targets either the C or U site.

(4) Add a non-hotstart, hear-tolerant polymerase i.e. native Thermus aquaticus DNA polymerase. Do not add dNTPs.

(5) Add a heat stable ligase (Ampligase, Epicentre). Incubate at 65° C.˜2 minutes.

(6) The polymerase will cleave 3′ of the overhanging flap nucleotide (flap of endonuclease) revealing a 5′ phosphate group.

(7) The ligase creates a new phosphodiester bond between 3′ OH of the tag-bearing oligo and the initially C or U targeting oligo.

(8) Denature ˜95° C., 1 minute. Cycle between 65° C. and 95° C. up to 500 times.

(9) Remove unligated primers (treat with ExoSAP-iT, microcon filtration, magnetic bead cleanup etc.) or proceed straight to preamp. Oligos will not amplify DNA in the PCR well if they target the same strand. In any case, ExoSap treat after PreAmp.

(10) Dilute sample ˜1:10.

### Using a Fluidigm Chip:

(1) Detect amplicons and perform amplicon melt using Eva or LC green using a digital PCR or dynamic array.

(2) Look at the DNA melt date.

(3) Use the Tm differential PCR products to detect rare mutant or methylated DNA nucleotides.

(4) High Tms indicate the original DNA sequence contained a methylated C at the oligonucleotide ligation junction.

(5) Low Tms indicate the original DNA sequence contained a normal C at the oligonucleotide ligation junction.

(6) Count amplicons in each panel or between panels. This permits an excellent estimate of the % methylation (or SNP variants) present at a specific targeted locus.

(7) Note: multiple loci can be target-specific ligated at a single time. PreAmp using a common tag primer and a single target specific primer.

(8) Amplicons may also be directly sequences if appropriate tag sequences are appended to primers.

Other variations, such as the use of padlock-probe type primers, are described in the method titled: “Preamplification and amplification methods based on target-specific ligation via LCR/LDR (ligase chain and ligase detection reaction) followed by PCR.”

### Results Using Tm Enhancing Oligos to Detect Rare SNPs (or Methylation Sites)

As an example of the utility of this approach, the SNP responsible for the clynically-relevant EGFR T790M mutation (mediating resistance to anti-cancer medication) was examined using Tm enhancing tag primers and a ligase detection assay in a DID chip. See FIG. 5B. Data derived from the procedure shown in FIG. 5B is shown in FIGS. 5C-5D.

If targeting a methylated C(C does not alter after bisulphite treatment) the overhanging flap nucleotide would be C. If targeting the normal C (normal C is deaminated to a U) the overhanging flap nucleotide would an A.

Amplify using a common tag primer (Tag 1 in image) and a common reverse primer. Determine methylation or SNP amplicon difference by examining Tm difference.

### REFERENCES

Backdahl. L., M. Herberth, et al. (2009). “Gene body methylation of the dimethylarginine dimethylamino-hydrolase 2 (Ddah2) gene is an epigenetic biomarker for neural stem cell differentiation.” Epigenetics 4(4): 248-254.
Broske, A. M., L. Vockenstanz, et al. (2009). “DNA methylation protects hematopoietic stem cell multipotency from myeloerythroid restriction.” Nat Genet. 41(11): 1207-1215.
Calladine, C. R., H. R. Drew, et al. (2004). Understanding DNA: the molecule & how it works. San Diego, Academic Press.
Eckhardt, F., J. Lewin, et al. (2006). “DNA methylation profiling of human chromosome 6, 20, and 22.” Nat Genet. 38(12): 1378-1385.
Fanelli, M., S. Caprodossi, et al. (2008). “Loss of pericentromeric DNA methylation pattern in human glioblastoma is associated with altered DNA methyltransferases expression and involves the stem cell compartment.” Oncogene 27(3): 358-365.
Farthing, C. R. G. Ficz, et al. (2008). “Global mapping of DNA methylation in mouse promoters reveals epigenetic reprogramming of pluripotency genes.” PLoS GENET 4(6):e1000116.
Frommer, M., L. E. McDonald, et al. (1992). “A genomic sequencing protocol that yields a positive display of 5-methylcytosine residues in individual DNA strands.” Proc Natl Acad Sci USA' 89(5): 1827-1831.
Goossens. E. M. De Rycke. et al. (2009). “DNA methylation patterns of spermatozoa and two generations of offspring obtained after murine spermatogonial stem cell transplantation.” Hum Reprod 24(9):2255-2263.
Li, C., Z. Chen, et al. (2009). “Correlation of expression and methylation of imprinted genes with pluripotency of penhenogenetic embryonic stem cells.” Hum Mol Genet. 18(12): 2177-2167.
Lister, R., M. Pelizzola, et al. (2009). “Human DNA methylomes at base resolution show widespread epigenomic differences.” Nature 462(7271):315-322.
Shen, X., Y. Liu, et al. (2008). ‘EZH1 mediates methylation on histone H3 lysine 27 and complements EZH2 in maintaining stem cell identity and executing pluripotency.” Mol Cell 32(4): 491-502.
Tate, C. M., J. H. Lee. et al. (2009). “COX linger protein 1 contains redundant functional domains that support embryonic stem cell cytosine methylation, histone methylation, and differentiation.” Mol Cell Biol 29(14): 3817-3831.
Vaid, M. and J. Floros (2009). “Surfactant protein DNA methylation: a new entrant in the field of lung cancer diagnostics? (Review).” Oncol Rep 21(1): 3-11.
Weisenberger, D. J., B. N. Trinh, et al. (2008). “DNA methylation analysis by digital bisulfite genomic sequencing and digital MethyLight.” Nucleic Acids Res 36(14): 4689-4698.
Xi, S., T. M. Geiman, et al. (2009). “Lsh participates in DNA methylation and silencing of stem cell genes.” Stem Cells 27(11): 2691-2702.

### Example 6

### Use of Pre-Amplification Methods to Overcome Sampling Limitations of Highly Precise Measurements Such as Non-Invasive Detection of Aneuploidy, CNV and Mutation Dosage

### Problem Statement:

Fetal aneuploidy detection from plasma: Plasma contains a limited concentration of target nucleic acids (DNA, methylation markers, SNPS, RNA etc). It is also necessary to analyze multiple targets in this limited amount of sample. Dividing the sample will reduce the number of target per assay and increase sampling error. Multiplexing will be necessary, which is traditionally viewed as being in direct conflict with the requirement for highly precise quantitative analysis.

In our approach to measure relative chromosome number (RCN) by microfluidic PCR, an additional observation has been mae: for the most precise measurements it is necessary to measure tens of thousands of target molecule to oversome the sampling errors which will otherwise mask the difference that have to be detected.

Normal samples to not have the minimum number of DNA molecules, nor the requisite concentration necessary for optimal quantitation using any of a variety of techniques including digital PCR, BEAMING, or next generation sequencing, to name a few. Hence, the direct simultaneous detection of multiples targets (chromosome, multiple loci per chromosome, multiplexing, etc.) is technically very challenging.

### Solution:

To overcome the sampling hurdle, we have developed a pre-amplification protocol which amplifies all targets of interest in the sample. This boosts the copy number and concentration for all targets, so as to allow analysis of all targets—in parallel, or together (e.g. introducing common tags in the pre-amp process).

Pre-amplification reduces the sampling error per target, as all copies of this sample can be analyzed.

Pre-amplification produces very high copy numbers per target and highly concentrated sample.

Multiple targets can be pre-amplified in parallel. By assaying multiple loci per chromosome for example, one reduces the sampling error for the chromosome dosage.

By pre-amplifying, the input of targets into the digital analysis can be adjusted such that the quantification has maximal possible precision.

5′ tagging can be a pert of pre-amplification and actually also be used as a stand-alone procedure to combine multiple targets.

### Scope:

Similar challenges exist for mutation detection by mutation dosage in pregnancy and cancer, and therefore this approach can be used in these contexts.

### Example 7

### Use of Pre-Amplification and Digital PCR for the Enhanced Detection and Quantification of (Fetal) Aneuploidy, Point Mutations and SNPs

### Problem Statement

Detection of point mutations (SNP) is a major challenge in samples where the sequence variant is a minority in comparison to the other allele. Currently, optimized methods may achieve sensitivities below 1%. However, quantification of mutations at these levels is rarely achieved with high precision and accuracy, also due to the low total number of mutated sequences in a sample. In pregnancy plasma e.g. (similar to plasma of cancer patients), it would be advantageous to quantify the number of mutated vs non-mutated sequences, especially if one aims to determine if the mutation has been passed on from the mother (and/or the father) to the fetus.

For fetal aneuploidy detection by using digital PCR (including microfluidic dPCR and emulsion PCR), it is desirable to amplify the number of target molecules without bias in order to achieve precise quantification. This includes the amplification of multiple different targets. We were the first to show that PCR based multiplex PCR can actually be reproducible enough to meet this requirement not just for alleles (which Is relatively easy, as the same primers amplify both alleles) but also for different loci.

### Solution:

Perform a number pre-amplification cycles (PCR, but also other methods possible) of the whole sample to amplify the number of copies per target sequence (in general this amplification is not allele specific, but aims to retain the proportion between the alleles and different target loci). If more than one sequence (locus) is of interest, this pre-amplification can be performed in multiplex (>10 targets, even 100 to 10,000). After pre-amplification primers are in general removed (enzymatic clean-up by exonuclease I, diluted below active levels etc.) and the concentration of a sample is adjusted to the desired copy number for measurement by digital PCR.

For determination if the fetus carries the mutation for which the mother is carrier, the ratio of normal vs mutated allele in the plasma of the mother is determined: if r=1.00, the fetus also carries the mutation. If the ratio (mutation/wild type allele) is smaller than 1.00, the fetus does not carry the mutation. If the father was also a carrier of the same mutation and the fetus has two copies or the mutation, the ratio will be greater than 1.00.

The accuracy of the quantification depends on certain parameters:

Amount of plasma DNA used for the test. More DNA reduces sampling error of pre-amplification.

Perecentage fetal DNA in plasma DNA: higher fetal DNA percentage means that the difference to detect is greater.

Number of panels used for the quantification. The greater the number of reactions used to quantify both alleles, the greater the precision of the determined ratio.

There are many methods possible to distinguish the two (or more) alleles in digital PCR: TaqMan PCR (dual labeled hydrolysis probes), molecular beacons, allele-specific PCR methods, High Resolution Melting analysis (HRM) et al.

### Possible Variations and Modifications:

LCR, with many possible probe designs (tagged, circularized by ligation, FLAP, etc).

LDR, with many possible probe designs (tagged, circularized by ligation, FLAP, etc).

Using universal tags for preamp, universal or common tags (common=same tag for a group of targets, e.g. per each chromosome).

Allele-specific pre-amplification.

Tagged pre-amplification

Pre-amplification for low number of cycles (2-5 to 10) to increase the amount of sequence of interest without affecting the ratio between two alleles of the same locus with such precision, that differences below 10% in copy number between alleles or loci are actually detectable.

Use or a reference value obtained from multiple samples to normalize the measured ratio between different loci.

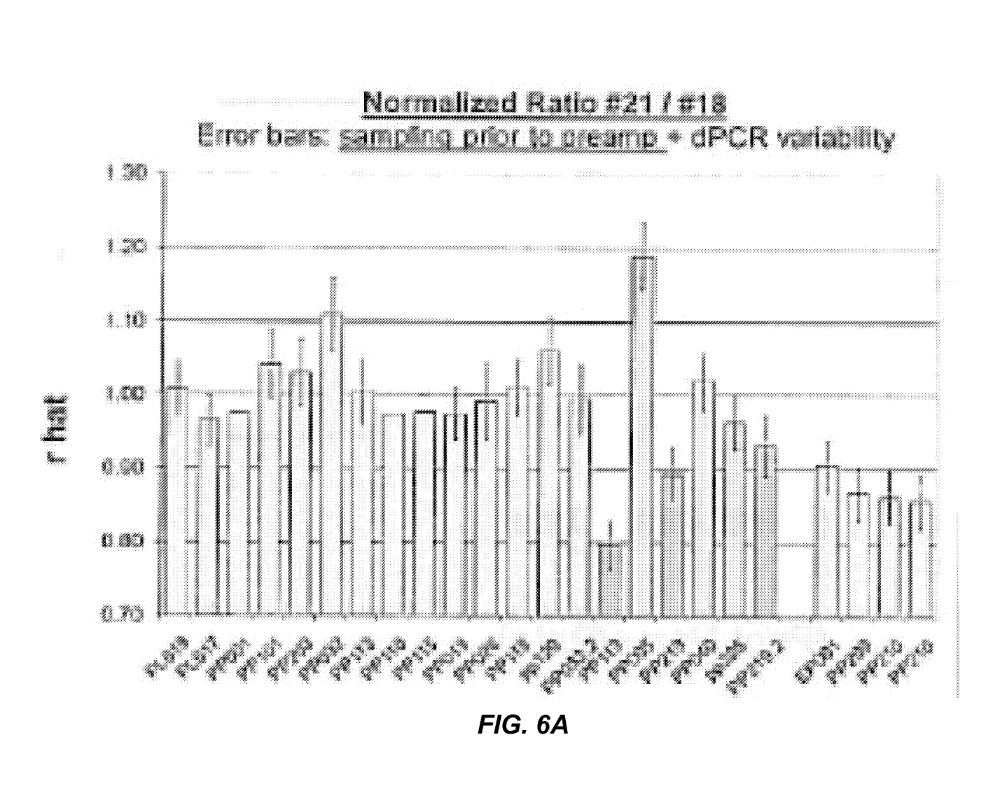

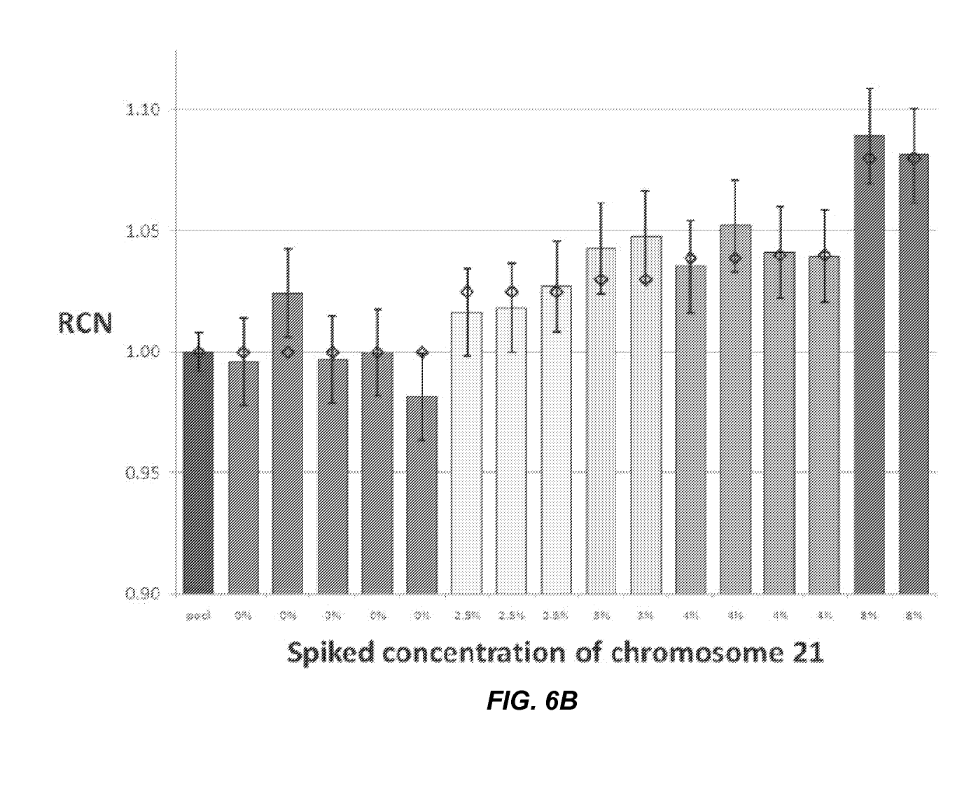

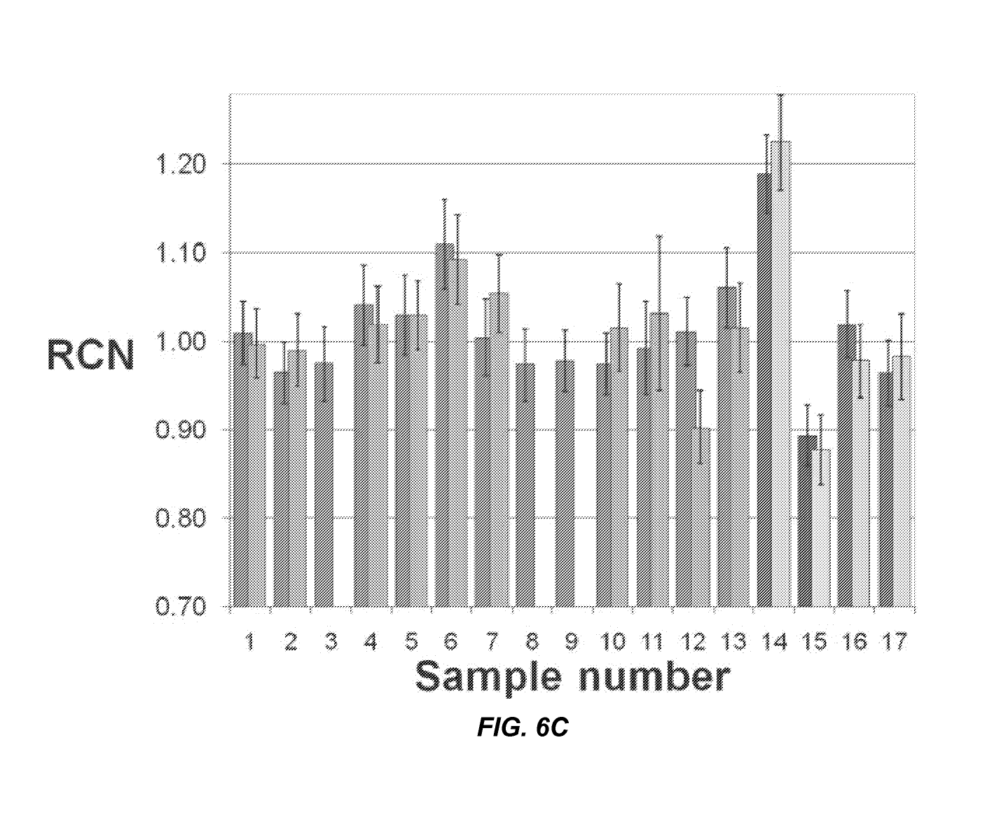

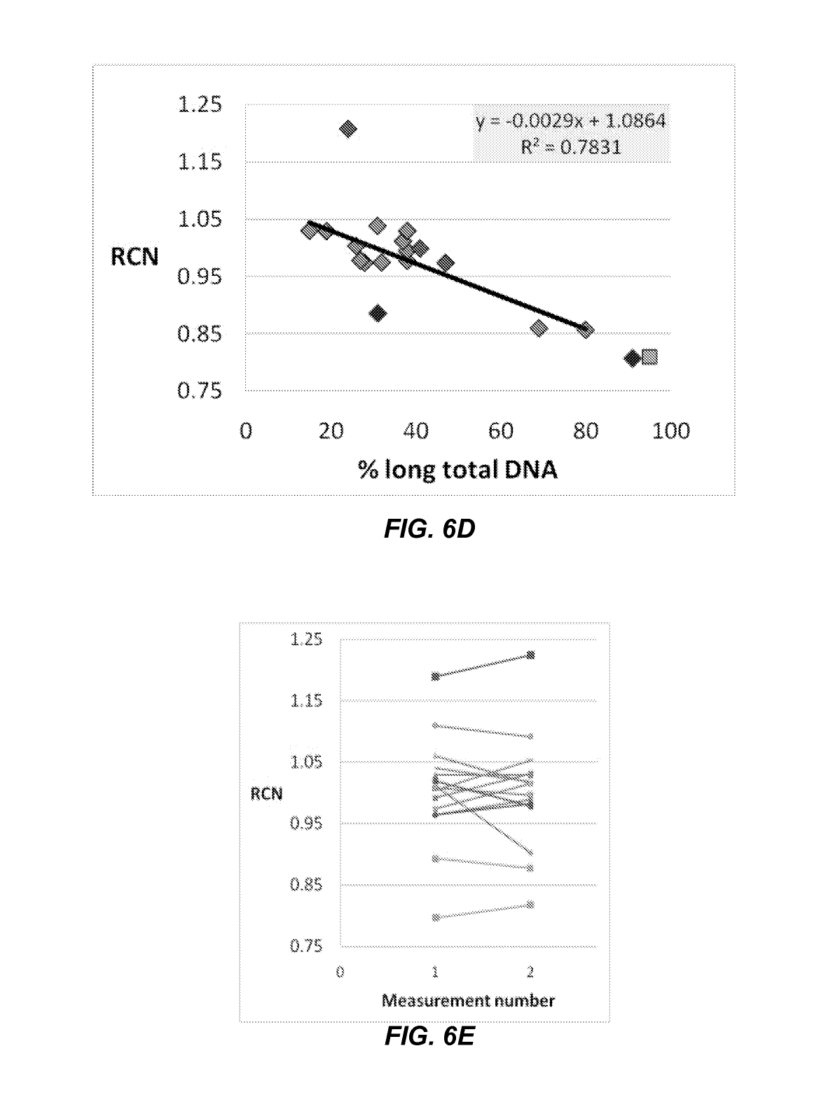

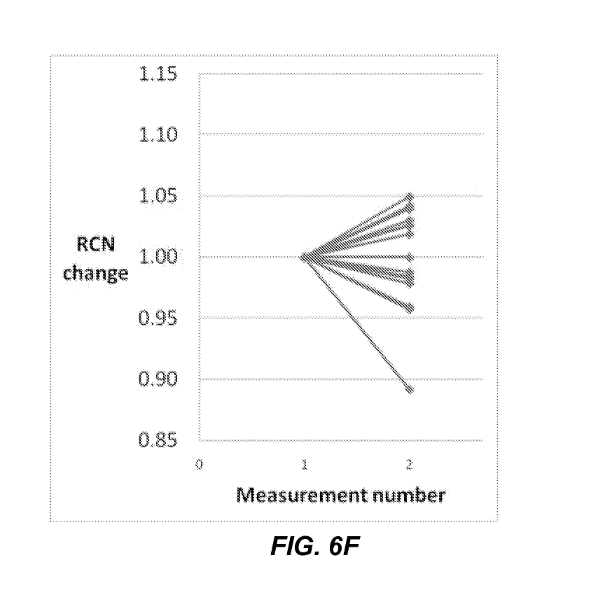

The results of an initial study of this approach to measure the relative copy number (RCN) between chromosomes 21 and 18 is shown in FIG. 6A.

### Non-Invasive Detection of Fetal Aneuploidy by Digital PCR

Background:

Measuring differences in chromosome dosage by using molecular counting has been suggested for the non-invasive prenatal detection of fetal aneuploidy. Measuring the relative copy number (RCN) between chromosomes 21 and 18 by digital PCR (dPCR) can be utilized for the non-invasive detection of fetal aneuploidy was investigated.

The method demonstrates the non-invasive prenatal detection of fetal aneuploidies using dPCR and cell-free DNA obtained from the plasma of women early in their pregnancies. The method utilized a high density 48.770 Digital Array™ integrated Fluidic Circuit (IFC), which permits the highly accurate determination of RCN by partitioning a single sample into as many as 36,960 reactions and after thermal cycling counting chambers positive for the targets of interest. On the right of the IFC are 48 sample wells into which the sample/PCR mix is added. On the left are two hydration inlets for addition of water (to prevent dehydration of PCR chambers during thermal cycling). The elastomeric core is in the center of the IFC. This is a network of fluid lines and reaction chambers into which the reaction mix is partitioned by applying and releasing pressure, thereby opening and closing NanoFlex™ valves.

The method is universally applicable to all patients by targeting non-polymorphic sequences, relatively inexpensive in comparison to high throughput sequencing and the entire assay can be completed in a single day.

Digital counting approaches have recently been the main focus of non-invasive prenatal diagnostic research towards aneuploidy detection [4]. The groups of Quake and Lo used next generation sequencing to discriminate fetal aneuploidies from unaffected pregnancies by counting sequence reads per chromosome [5-7]. All DNA fragments in a sample are targeted for library generation, and tens of thousands per chromosome are sequenced and counted. The high number of targets allows a measurement with very small error and it is possible by the sheer number of counted molecules to detect relative copy number (RCN) differences of only a few percent, as is the case in maternal plasma when the fetus has an aneuploidy. Universal applicability to all pregnancies and the precision to detect trisomy in samples with a low fetal fraction make digital counting by sequencing a viable alternative to invasive methods.

Such a DNA counting based approach carries great conceptual benefit for the non-invasive detection of fetal aneuploidies, since one can select target sequences across the entire chromosome of interest, without being restricted to specific genes. This kind of approach can be universally applied to any patient, as opposed to other DNA- and RNA-based approaches such as those which target specific SNPs. The advantage of dPCR in a nanofluidic format over sequencing is the very simple workflow where results can be obtained in a single working day. DNA is extracted from plasma, combined with fluorescent PCR assays for each chromosome of interest, and loaded into the nanofluidic chip for thermal cycling and subsequent counting. In the chip, the bulk sample-reaction-mixture is divided into thousands of individual reactions near the limiting dilution of the sample [12]. As the measurement error is a function of both, the number of molecules and the number of chambers [7], an increased counting of the number of reactions in the dPCR nanofluidic format is desirable to obtain the same precision as sequencing, but with a single day workflow.

Improved precision has been realized through development the present methods which optimally use the digital format of the 48.770 Digital Array™ chip or other digital PCR formats. As described herein, the entire 36,960 parallel real-time PCR reactions of a single chip was used for the analysis of chromosome 21 and chromosome 18 targets for a single sample, showing that with microfluidic dPCR it is possible to quantify DNA sequences for the non-invasive prenatal detection of fetal chromosomal aneuploidy.

This study was performed in a retrospective manner using maternal plasma samples collected with informed consent under approval by the Institutional Review Board of the Polish Mother's Memorial Hospital Research Institute. Peripheral venous blood was collected from each patient by venipuncture of an antecubital vein into a Sarstedt vacuum collection system (Each S Monovette contains sufficient potassium EDTA to achieve a concentration of 1.2-2 mg EDTA/ml blood after collection). The blood samples were obtained prior to amniocentesis, which was performed because of the increased risk of fetal aneuploidy based on biochemistry, ultrasound, or because of maternal age or family history.

Plasma was obtained by centrifuging the blood at 1600 g for 10 minutes and separating the supernatant from the cell pellet and then frozen at −20° C.

Cell-free DNA was extracted from 5 ml plasma and eluted into 150 μl elution buffer using the circulating nucleic acids kit (Qiagen, Germany) according to the manufacturer's instructions. The DNA samples were then dried under vacuum (speedVac) and dissolved in 50 μl water.

Small aliquots of the plasma DNA samples were amplified in one panel of the 12.765 Digital Array™ chip to determine concentrations of total and fetal DNA using 45 base pair long PCR assays for a chromosome 18 sequence and DYS14 on the Y chromosome. The forward primers of these short assays were tagged with universal template sequences to permit two-color detection with dual labelled hydrolysis probes [13, 14]. The proportion of detected long fragment DNA was assessed by targeting 188 bp and 194 bp long sequences in a second panel, the forward primers sharing the same target sequence with the short assay.

The DNA from pregnancy plasma samples was pre-amplified with tagged primers for chromosome 18 and 21 sequences. The pre-amplification was necessary as the concentration and total copy number of DNA extracted from plasma is sometimes too low to be quantified directly on the microfluidic chip. Approximately 10′000 single stranded copies of total DNA per sample were subjected to 2 cycles of tagging and 15 PCR cycles of pre-amplification using tagged primer pairs specific for 50 base pair sequences on chromosomes 21 and 18. Starting with 10′000 molecules per target 32 Million single strands of pre-amplification product were expected. After pre-amplification primers were removed with ExoSAP-IT (USB) and further diluted products were stored at −20° C.

A high-density Digital Array IFC that was programmable to form three different input configurations was used in this method. In the first configuration, 48 individual samples could be measured over 770 reaction chambers each, in the second, a single sample could be measured over the entire chip (770×48=36,960 chambers) and in the third configuration 12 individual samples could be measured over 3080 reaction chambers (770×4). Each configuration is made possible by the selective opening and shutting of the valves within the chip. The results presented here were generated using the single sample configuration, i.e., a single sample was be partitioned over the entire 36,960 reaction chambers of the chip. The pre-amplified samples were analyzed using one (in exceptions half) 48.770 Digital Array™ IFC per sample. Sample input was adjusted to obtain an estimated 200 to 700 positive PCR reactions per panel of 770 reactions (230-1800 targets). Duplex real-time PCR detection of pre-amplification products for chromosome 21 and 18 sequences was performed under standard PCR conditions using primers specific to the tags introduced in the pre-amplification and dual labelled hydrolysis probes stretching over the junction of the pre-amplification primers

For 3 samples 4 replicate pre-amplifications were performed, each with one quarter of the DNA obtained from 5 ml plasma (4500-6000 copies per pre-amplification). One chip was used for the analysis of each pre-amplification and the average ratio of four chips used to determine the RCN.

Pre-amplification product of plasma DNA from a normal (euploid) pregnancy sample was prepared with different amounts of chromosome 21 spike (genomic DNA (Coriell PN NA13783) pre-amplified with chromosome 21 primers only). Sample and spike material were each quantified using dPCR on the 48.770 Digital Array™ IFC. From this measurement, the effective fetal concentration of the mixed sample was determined.

Total counts for chromosome 18 and 21 of a sample were converted into estimated target molecules and an estimated ratio between chromosomes 21 and 18 as described earlier [8]. The measurement error in the chip was combined with the sampling error based on the number of copies into the pre-amplification (SE=\sqrt{n}/n) by adding the square of the standard deviations and then taking the square root of the sum. Any additional variability was neglected.

The median “raw” ratio of chromosome 21 vs. chromosome 18 from the euploid pregnancy plasma samples and used it as a normalization factor to correct for the detection bias between the two targets. The observed ratio of a sample was divided by the normalization factor to give the relative copy number (RCN) of chromosome 21 vs. Chromosome 18. To discriminate trisomy 21 or trisomy 18 from normal The following criteria were used: If the CI around the normalized RCN does not include 1.00, the measurement is indicative of a suspected fetal aneuploidy.

A normal pregnancy plasma sample pre-amplified for chromosomes 18 and 21 was used for titration experiments into which different amounts of a chromosome 21 spike were titrated. There is an inherent variability in measurement due to sampling, so the purpose of the titration was to determine the minimum fetal concentration required to distinguish between normal and trisomy 21. Different amounts of spike material were added to the normal sample, creating an expected increase in chromosome 21 copy of 2.5%, 3%, 4%, and 8% (corresponding to 5%, 6%, 8% and 16% fetal DNA concentration for a trisomy sample). The un-spiked sample was analyzed in 5 chips with the same input concentration. The individual chips counts were summed and the 21/18 ratio (n=240 panels) determined. The raw ratio calculated by summing the counts of five chips (184′800 reactions) is 0.959 with 95% confidence interval boundaries of 0.952 and 0.967. By normalizing measurements using the pooled reference, the 95% confidence intervals of a sample's measured RCN should not overlap with these boundaries to be classified as a trisomy. All chips of the normal sample fell within the normal range, one of three tests of a 2.5% spike sample and all spiked samples with 3% or more additional chromosome 21 could be classified as trisomies (FIG. 6B). This corresponds to a fetal proportion of 6% or higher in maternal plasma.

A small amount of each pregnancy plasma DNA sample was analyzed in two panels of the standard 12.765 Digital Array™ IFC to determine sample concentration and as quality control. In one panel the PCR assays used were 45 bp long. In male pregnancies an estimate was made of percentage and of absolute copy number of fetal DNA. The additional measurement of 190 bp assays in a second panel was used to assess the presence of long fragment DNA.

A strong bias in the determined RCN for genomic DNA and pregnancy samples containing a large proportion of long DNA (FIG. 6D) was observed. In the original study cohort this affected one euploid plasma DNA sample (RCN=0.86) and one trisomy 18 sample (RCN=0.80) with more than 50% of detected long total DNA. After identifying the effect of a high proportion of long fragment DNA, the analysis of the normal pregnancy sample and of another sample with high percentage of long DNA was repeated. In both these samples the RCN bias was confirmed (RCN=0.86 and 0.87). Consequently samples were excluded with more than 50% of long fragment total DNA retrospectively.

Pregnancy Plasma

In total, 13 normal pregnancy samples and 4 samples with an aneuploidy fetus (trisomy 21 or trisomy 18) were included. The median ratio of the normal samples was 0.93. To compensate for the systematic bias between the two target sequences, the measured ratio was normalized by this value. For 11 of 13 normal samples the RCN between chromosome 21 and 18 was within 1.00±0.05, in ten cases the 99% CI included 1.00 (i.e. sample not determined to be abnormal). The CI was determined without accounting for the variability of the pre-amplification. As such, the consistency of the determined ratios was reassuring in that the variability of the pre-amplification is so low, that it allows the very precise measurement of relative copy number (RCN). Only one euploid sample with a very high fetal DNA concentration was clearly outside of this range (24%, RCN=1.11). In a second analysis including pre-amplification this sample tested normal (RCN=1.00).

For one trisomy 21 sample the RCN was clearly indicative of trisomy 21 (RCN=1.19) and the RCN of the tested trisomy 18 samples were clearly indicative of trisomy 18 (RCN=0.89). Two trisomy 21 samples tested within the normal range (1.02 and 0.96). The fact that they remained undetected could be explained by a low percentage of fetal DNA; however, the percentage of fetal DNA was not determined since these fetuses were female. Interestingly, these two samples had the highest proportion of long fragment DNA of all samples that were included in the analysis. Thus a small bias caused by long DNA could have contributed to the result.

Blinded Re-Test of Pre-Amplified Samples

To assess the stability of the measurements and the stability of the pre-amplified samples, the analysis of 16 pre-amplified samples was repeated in a blinded experiment. The three samples that had already been analysed as 4 replicate pre-amplifications on 4 chips were not re-analyzed. Sample identities were blinded prior to testing. Compared to the initial analyses, the 99% CIs in all but one sample overlap and the RCN of the 2^{nd} chips are within ±5% of the first (FIG. 6E-6F). For one pre-amplified sample a decrease of more than 10% of the RCN was observed, and this discrepancy persisted after re-testing. The consistency between first analysis and blinded re-test of the same pre-amplification products confirms the initial results (FIG. 6C). The stability of the pre-amplified product makes it possible to retest the pre-amplified material if necessary or add additional chips for counting more reactions of borderline samples.

Of the normal samples that were tested twice, at least one of the two results included 1.00 in the 99% CI. Combining the initial and repeat measurements results improves the test performance, the average RCN of the normal samples lie between 0.974 and 1.038 (excluding the sample with discrepant results in the second test).

Detection of copy number differences below 5% by counting positive reactions can be performed. In titration experiments it was possible to consistently detect a 3% copy number difference of one target against another, a difference that corresponds to a trisomic pregnancy plasma sample with 6% fetal DNA.

The measurements of the RCN for normal samples were in most cases close to “1.00”. Blind reanalysis of the pre-amplified samples confirmed the stability of measurements and pre-amplified material. In the included 13 normal samples only one sample was a clear “false positive”. The sample had a very high fetal concentration (25%). While this may have affected the result, this was not the case in a repeat experiment. One pregnancy with trisomy 18 and one with trisomy 21 were clearly distinguished from normal (euploid) pregnancy outcome while two trisomy 21 cases were not. For the detected trisomy 21 pregnancy sample a fetal DNA content of 7.4% was measured. In the two undetected trisomy 21 samples fetal DNA was not quantified (female fetuses) and both had a relatively high proportion of long fragment DNA (>40%). This emphasises the importance of precise quantification of fetal DNA—when the fetal proportion is very low a higher number of molecules will have to be counted.

The development of the described high density nanofluidic chip makes the NIPD of fetal aneuploidy using maternal plasma technically feasible. In combination with the ability to detect higher fetal DNA levels by using short PCR assays [13], the high-density digital PCR chips improve the sensitivity to measure. Pre-amplification was used to increase the number and concentration of target molecules to the necessary levels as well as to append tags to target sequences, which enabled the use of a target specific hydrolysis probe for detection of 50 bp target sequences. The multiplex pre-amplification for a limited number of PCR cycles has in the past years facilitated the (quantitative) analysis of a number of applications [20-22], and it can be a useful tool even for the detection of copy number differences below 5%.

Several targeted approaches for the NIPD of fetal aneuploidies using cell free nucleic acid have been identified in the past years. None has thus far been followed up by successful clinical validation studies and implementation. Polymorphic markers in DNA and RNA and methylation specific analysis of DNA have the advantage that the fetal material is distinct from the maternal background—even if, as is the case for SNPs, only by one nucleotide [23-25]. However, such approaches have to “battle biology” (number of possible targets, sample concentration and stability, heterozygosity rate and informative of SNPs per sample), while the workflow and analysis are inherently extensive.

Next generation sequencing has recently been used successfully by two groups for the detection of trisomies 18 and 21 without false results [5-7]. By counting molecules, the sequencing approach is closely related to the use of high density dPCR which determines target copy numbers by counting positive reactions. The final numbers of sequencing and dPCR are in a similar range, Fan and Quake sequenced about 66,000 chromosome 21 reads per sample, whereas nanofluidic dPCR at an optimal sample concentration yields approximately 80 to 85% positive chambers, which corresponds to 60 to 70,000 molecules.

This approach can be implemented in a clinical set-up: First, sample collection and processing should be optimal: a large volume of blood, at least 10 ml, should be drawn to obtain a large number of target molecules. To assure optimal sample quality and fetal DNA proportion, the sample will need to be centrifuged immediately after blood draw. QC analysis should be performed to measure total DNA concentration and to exclude samples with “contamination” by leukocyte derived long DNA. The fetal DNA percentage should be measured to determine the expected copy number difference in case of a trisomy and to identify samples with a very low percentage of fetal DNA. In case of a female pregnancy, an epigenetic or SNP based approach could be implemented. While a large number of SNPs would have to be tested, the SNP assays can be included in the pre-amplification reaction. The pre-amplification can be performed with the majority of a sample and the optimal sample input into the digital PCR analysis can be calculated from the QC data. In the case of a suspected aneuploidy, the pre-amplified sample would be retested with another chip to confirm the positive test result. Positive tested pregnancies could be treated as screening positive and referred to invasive diagnostic testing.

Another advantage of the molecular approach in comparison to the phenotype-based screening is the fact that the latter has a relatively narrow time-frame, outside of which the discriminatory power of the screening test is markedly reduced. This is a very practical issue, since the optimal time for ultrasound screening is only 3 weeks wide at most (11-14 wks). Even within this period the performance of the screening changes significantly. Organizational problems and the imprecision associated with correctly identifying the date of conception cause that many pregnant women come too late for this type of screening, which would not be an issue with the molecular approach. The time-frame of the DNA-based approach is limited only in early pregnancy, when the placenta produces too little free nucleic acids to enable discrimination. In case of an equivocal result the molecular test should in theory perform better at retesting, as opposed to phenotype-based screening.

### REFERENCES

Chiu, R. W., C. R. Cantor, and Y. M. Lo. “Non-invasive prenatal diagnosis by single molecule counting technologies.” Trends Genet. 25.7 (2009): 324-31.
Fan, H. C., et al. “Noninvasive diagnosis of fetal aneuploidy by shotgun sequencing DNA from maternal blood.” Proc. Natl. Acad. Sci. U.S.A 105.42 (2008): 16266-71.
Chiu, R. W., et al. “Noninvasive prenatal diagnosis of fetal chromosomal aneuploidy by massively parallel genomic sequencing of DNA in maternal plasma.” Proc. Natl. Acad. Sci. U.S.A 105.51 (2008): 20458-63.
Chiu, R. W., et al. “Maternal Plasma DNA Analysis with Massively Parallel Sequencing by Ligation for Noninvasive Prenatal Diagnosis of Trisomy 21.” Clin. Chem. (2009).
Dube, S., J. Qin, and R. Ramakrishnan. “Mathematical analysis of copy number variation in a DNA sample using digital PCR on a nanofluidic device.” PLoS. One. 3.8 (2008): e2876.
Lun, F. M., et al. “Microfluidics digital PCR reveals a higher than expected fraction of fetal DNA in maternal plasma.” Clin. Chem. 54.10 (2008): 1664-72.
Fan, H. C. and S. R. Quake. “Detection of aneuploidy with digital polymerase chain reaction.” Anal. Chem. 79.19 (2007): 7576-79.
Fan, H. C., et al. “Microfluidic digital PCR enables rapid prenatal diagnosis of fetal aneuploidy.” Am. J. Obstet. Gynecol. 200.5 (2009): 543-47.
Sykes, P. J., et al. “Quantitation of targets for PCR by use of limiting dilution.” Biotechniques 13.3 (1992): 444-49.
Sikora, A., et al. “Detection of Increased Amounts of Cell-Free Fetal DNA with Short PCR Amplicons.” Clin. Chem. (2009).
White, R. A., III, et al. “Digital PCR provides sensitive and absolute calibration for high throughput sequencing.” BMC. Genomics 10 (2009): 116.
Bhat, S., et al. “Single molecule detection in nanofluidic digital array enables accurate measurement of DNA copy number.” Anal. Bioanal. Chem. 394.2 (2009): 457-67.
Angert, R. M., et al. “Fetal cell-free plasma DNA concentrations in maternal blood are stable 24 hours after collection: analysis of first- and third-trimester samples.” Clin. Chem. 49.1 (2003): 195-98.
Chan, K. C., et al. “Effects of preanalytical factors on the molecular size of cell-free DNA in blood.” Clin. Chem. 51.4 (2005): 781-84.
Chiu, R. W., et al. “Effects of blood-processing protocols on fetal and total DNA quantification in maternal plasma.” Clin. Chem. 47.9 (2001): 1607-13.
Li, Y., et al. “Size separation of circulatory DNA in maternal plasma permits ready detection of fetal DNA polymorphisms.” Clin. Chem. 50.6 (2004): 1002-11.
Hu, Z., et al. “Exon-level expression profiling: a comprehensive transcriptome analysis of oral fluids.” Clin. Chem. 54.5 (2008): 824-32.
Qin, J., R. C. Jones, and R. Ramakrishnan. “Studying copy number variations using a nanofluidic platform.” Nucleic Acids Res. 36.18 (2008): e116.
Spurgeon, S. L., R. C. Jones, and R. Ramakrishnan. “High throughput gene expression measurement with real time PCR in a microfluidic dynamic array.” PLoS. One. 3.2 (2008): e1662.
Lo, Y. M., et al. “Digital PCR for the molecular detection of fetal chromosomal aneuploidy.” Proc. Natl. Acad. Sci. U.S.A 104.32 (2007): 13116-21.
Lo, Y. M., et al. “Plasma placental RNA allelic ratio permits noninvasive prenatal chromosomal aneuploidy detection.” Nat. Med. 13.2 (2007): 218-23.
Tsui, N. B., et al. “Non-invasive prenatal detection of fetal trisomy 18 by RNA-SNP allelic ratio analysis using maternal plasma SERPINB2 mRNA: a feasibility study.” Prenat. Diagn. 29.11 (2009): 1031-37.
Chiu, R. W., et al. “Maternal Plasma DNA Analysis with Massively Parallel Sequencing by Ligation for Noninvasive Prenatal Diagnosis of Trisomy 21.” Clin. Chem. (2009).

Supplementary Information

### QC Protocol for Testing Plasma DNA Preparations Using ZCCHC2 46/DYS45 and ZCCHC2 194/DYS188

This protocol was used for quantification of total DNA, male DNA and the determination of the percent fetal in samples with a male fetus. Also the distribution of the DNA target molecules into two size ranges was assessed. Six samples were analyzed per chip. The ZCCHC2 assays target the ZCCHC2 gene located on chromosome 18. The DYS14 assays target multiple copies on the Y chromosome and should only give a positive reaction with pregnancy plasma DNA when the fetus is male. Based on experiments with fragmented male DNA, the DYS14 assays will detect approximately 30 copies per Y chromosome. This number has been used to convert the number of targets of DYS14 into the number of targets of detected Y chromosome copies.

Oligonucleotides

Universal Target tag sequences of forward primers are underlined.

ZCCHC2_TQ1 F
TACCTGCGCTGTGGCCAATCGAATAAAACACACAGTACCGCGCAGAG
ZCCHC2_46 R
CAGCACTGATGTAAGAGGTGCTG
TQ1 Probe
5′ CAL Fluor Orange 560-
ATTCGATTGGCCACAGCGCAGGTA-3′ BHQ
DYS14 TQ2 F
AAGCTCAGTCATTTCCAGGTGTGCGAAAAGGGCCAATGTTGTATCCTTCT
C
DYS14_45 R
ACTAGAAAGGCCGAAGAAACACT
TQ2 Probe
5′ FAM-TCGCACACCTGGAAATGACTGAGCTT-3′ BHQ-1
ZCCHC2 F
ACACACAGTACCGCGCAGAG
ZCCHC2-194 R
GGTCCAGGCATTGGATTAGGAT
ZCCHC2 PB
5′ CAL Fluor Orange 560-
CAGCACCTCTTACATCAGTGCTGTGG-3′ BHQ
DYS_F
GGGCCAATGTTGTATCCTTCTC
DYS_188 R
CGCATGCAGGACAATAGTACCC
DYS14 PB
5′ FAM-TGTTTCTTCGGCCTTTCTAGTGGAGAGG-3′ BHQ-1

Preparation of 10× Duplex Assay Mixes

“45 By Assay”: Z46/D45

| Component | Conc in 10x (μM) |
|---|---|
| ZCCHC2 TQ1 F | 1 |
| ZCCHC2_46 R | 9 |
| TQ1 Probe (CalO) | 2 |
| DYS14 TQ2 F | 1 |
| DYS14_45 R | 9 |
| TQ2 Probe (FAM) | 2 |
| Tween 20 | 0.25% |

“190 By Assay”: Z194/D188

| Component | Conc in 10x (μM) |
|---|---|
| ZCCHC2 F | 9 |
| ZCCHC2_194 R | 9 |
| ZCCHC2 PB CalO | 2 |
| DYS14 F | 9 |
| DYS14_188 R | 9 |
| DYS14 PB FAM | 2 |
| Tween 20 | 0.25% |

Protocol for Assay

(1) To 2.0 μl of sample was added 6.3 μl of DNA Suspension Buffer (Teknova P/N T0221).

(2) Sample was heated at 95° C. for 1 min to denature the DNA.

(3) The master mix/DA Sample loading solution mixture was prepared.

| | Per reaction (10 μl) |
|---|---|
| Gene Expression Master Mix | 5.0 μl |
| (Applied Biosystems, AB) | |
| DA Sample Loading Solution (Fluidigm) | 0.5 μl |

(4) To each 8.3 μl sample was added 13 μl of master mix/loading solution.

(5) 9 μl of this mix were combined with 1 μl of the 45 bp duplex assay mix (10×) and 9 μl with the 190 bp assay.

| Tube | 10X assay |
|---|---|
| 1 | ZCCHC2_46/DYS14_45 |
| 2 | ZCCHC2_194/DYS14_188 |

(6) 9.5 μl of each of the prepared reaction mixes were pipetted into one sample well of a 12.765 Digital Array™ chip (Fluidigm).

(7) The reaction reaction mixtures were loaded into the panels of the chip in the IFC Controller (Fluidigm).

(8) PCR on the BioMark System (Fluidigm) was performed using a cycling program with 2 min at 50° C., 10 min at 95° C. and 45 cycles with 1 min annealing/extension at 60° C., 1 min extension at 72° C. and 15 seconds denaturation at 95° C., with data acquisition at 72° C. every cycle. (a modified the PCR protocol to: 2 min at 50° C., 10 min at 95° C. and 45 cycles with 1 min annealing/extension at 60° C. and 20 seconds at 95° C., with data acquisition at 95° C. has also been used.)

Processing Of Chip Data

(1) We used a threshold range of 1-45 cycles. The quality threshold was lowered to 0.3 if necessary.

(2) We determined whether the threshold was set correctly by the software for the different assays and samples on the chip and set it manually if necessary. Care in choosing thresholds must be used so as not to set them too low and as a consequence pick up CalO in the Fam channel or Fam signal in the CalO channel.

(3) Once all of the panels had been checked the data was exported for analysis.

Calculation of Total Copies/ml Plasma and Copies/μl DNA in the Sample

The data derived from this test was be used to determine the % male fetal in a sample, the total DNA based on the 46 bp ZCCHC2 assay, as well as the distribution of the DNA in two size categories.

To determine the percentage of detected fetal DNA, the number of detected DYS14 targets is divided by 15 and by the number of targets for ZCCHC2.

To calculate the number of detectable copies for each assay, the number of targets in a panel determined by the software is divided by 0.459 to determine the number of targets in the 10 μl PCR mix (the total volume of reactions in a panel is 4.59 μl). As 0.845 μl sample (containing the DNA from 0.0845 ml plasma) are in each PCR mix, this is further divided by 0.0845 to determine the total number of targets per ml plasma. For the DYS14 assay, the number of DYS14 targets is divided by 30 to obtain the number of detected Y-chromosome copies per ml.

Pre-Amplification and Digital PCR on 48.770 Digital Array™ IFC

Description

This protocol is be used for determination of RCN of chromosomes 21 and 18. The #21 assay targets a 48 nt sequence located on chromosome 21, the #18 assay a 49 nt sequence located on chromosome 18.

Pre-Amplification Primers

In the primer sequences the tag sequence is underlined. In the amplicon sequences capital letters indicate the portions matching the primers, the bold sequence is used for the probe in the subsequent digital PCR.

#18 F
TCAATTCCAGGTGTGCGAAAGCTGTCAGGGCTGCAGGTA
#18 R
TGGACGAGCAACAGCACTATAAACCGAAGGTGTTGAGAG
AGACG
Amplicon
GCTGTCAGGGCTGCAGGTAgtgagtgcCGTCTCTCTCAACA
CCTTCGG
#21 F
TGCAACGAGTTAGTGGAACAGAATTGACCTGAAGTAGCA
TTTAGTTACCAAG
#21 R
ACAGCACAACTCGCAATTGAACCTGTGTGGAGTGGGCTG
T
Amplicon
TGACCTGAAGTAGCATTTAGTTACCAAGccACAGCCCA
CTCCACACAGG

Preparation of Pre-Amplification Reactions

The pre-amplifications were performed in 25 μl reactions using the TaqMan° PreAmp Master Mix (AB). If the volume was not sufficient for the DNA input, multiple reactions were performed and pooled after pre-amplification.

| | final concentration |
|---|---|
| TaqMan ® PreAmp Master Mix | 1X |
| Primers | 300 nM |
| tRNA | 2.4 μg/μl |
| DNA | |

The PCR pre-amplification was performed using the following cycling conditions: Two cycles with 10 min at 95° C., 1 min at 68° C., 1 min at 65° C., 4 min at 60° C. and 1 min at 72° C. followed by 15 cycles with 20 sec at 95° C. and 4 min at 72° C., then cooled to 4° C.

Dilution And Clean-Up of Pre-Amplification Products

The products were diluted 3-fold in Exo buffer and primers digested with ExoSAP-IT (USB) at 37° C. for 15 minutes and 60° C. enzymes inactivated at 80° C. for 15 minutes. After cooling to 4° C., the products were diluted 12.5-fold in water and frozen.

### Oligonucleotides of Digital PCR

TABLE 3

| | |
|---|---|
| F T18 | TCAATTCCAGGTGTGCGAAA |
| R T18 | TGGACGAGCAACAGCACTATAAA |
| F T21 | TGCAACGAGTTAGTGGAACAGAAT |
| R T21 | ACAGCACAACTCGCAATTGAA |
| #18 Probe | 5′ FAM-CTGCAGGTAGTGAGTGCCGTCTCTC-3′ BHQ |
| #21 Probe | 5′ CAL Fluor Orange 560- |
| | AAGTAGCATTTAGTTACCAAGCCACAGCCCA-3′ BHQ |

Preparation of dPCR Reactions

Digital PCR was performed in the 48.770 Digital Array™ IFC (Fluidigm) using the TaqMan° Gene Expression Master Mix (AB). When using the 1 sample configuration chip (R&D version), 50 μl of reaction mix was prepared and pipetted into 2 sample loading wells. When using the commercially available version of the chip, 5 μl of reaction mix was loaded into 48 sample wells. The input of pre-amplified sample was adjusted to yield between 200 to 700 positive reactions per panel. Thermocycling and processing of chip data were performed as described for the 12.765 Digital Array™ IFC.

TABLE 4

| | final |
|---|---|
| | concentration |
| GE Master mix | 1X |
| Primers | 900 nM |
| Probes | 200 nM |
| Sample Loading Reagent | 1X |
| Sample | |

### Example 8

### A Multiplexed Approach for Detection of Fetal Aneuploidies in Maternal Plasma

Trisomies 21 (Down's syndrome), 18 (Edwards syndrome) and 13 (Patau syndrome) are the most common fetal chromosomal aneuploidies of clinical importance. Amniocentesis and chorionic cillus sampling are conventionally used invasive techniques for the early detection of aneuploidies and, when properly performed, are both very accurate (˜100%), although they carry a risk of fetal loss and other complications. The finding that circulating cell-free fetal DNA is present in maternal plasma has made noninvasive detection of fetal aneuploidies possible. Since a trisomy 21 fetus will release more chromosome 21 sequences than any other chromosome to the maternal plasma, theoretically by comparing the concentrations of chromosome 21 sequences and those of another chromosome, we are able to detect an increase in chromosome 21 dosage.

Detection of fetal aneuploidies is in fact a copy number measurement problem. We have shown before that Fluidigm's digital array provides a new approach that can measure copy number much more accurately than any other technologies. Its value is more prominent in fetal aneuploidy study. For example at 10% fetal DNA concentration, the amount of chromosome 21 sequences compared to those of a normal chromosome in the plasma from a trisomy 21 fetus-carrying woman is only 5% more than that of a woman with a normal fetus. This small copy number difference cannot be detected by any conventional methods.

Since fetal DNA fragmentation is the result of an apoptosis-like event, it is unavoidable that some nucleotides are randomly missing. Therefore if we just focus on a single locus on each chromosome, it is very likely that the 21/18 ratio will fluctuate from sample to sample, making the reliable detection of the 5% difference impossible. Our own single locus (or even 2 to 3-locus) TaqMan-based experiments on plasma DNA have failed to deliver consistent results. We also experimentally shown that different loci are represented differently in the same maternal plasma DNA. To overcome this problem, we decided to analyze multiple loci on each chromosome so that the fluctuation of individual loci will be evened out.

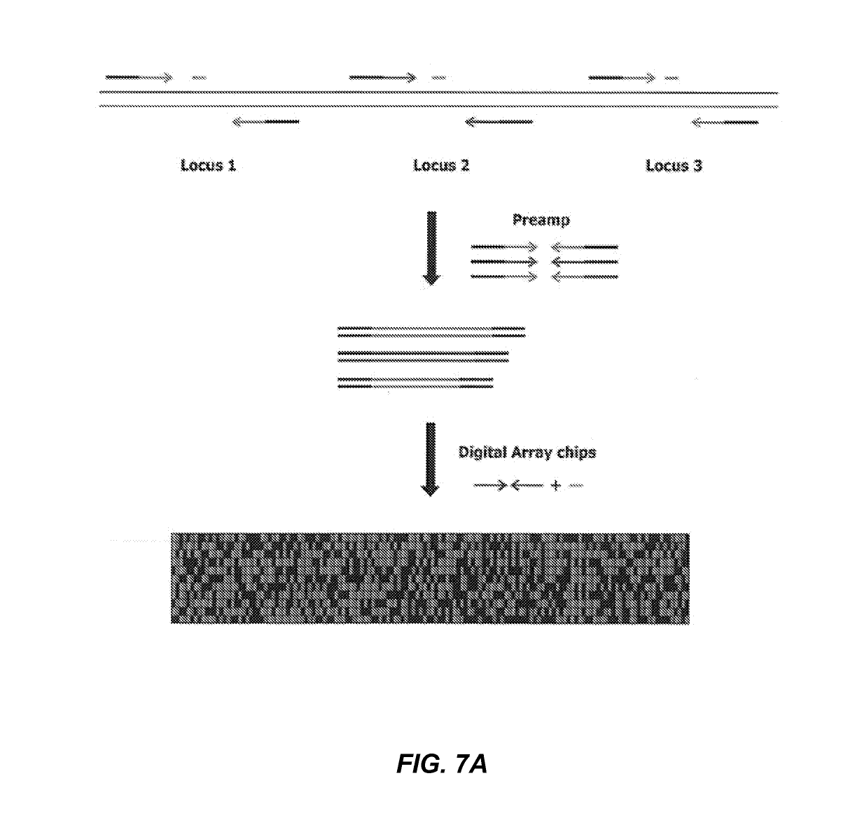

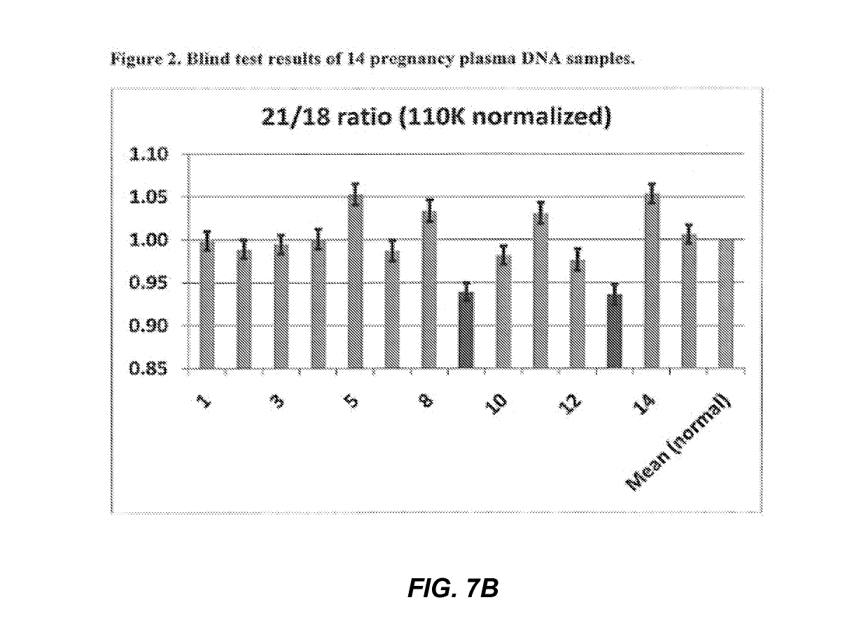

We have developed a multiplexed PCR-based approach to address this challenge (FIG. 7A). For the sake of simplicity, we detected amplicons using Locked Nucleic Acid (LNA) probes purchased either from Roche Applied Science (Universal Probe Library, Roche Applied Science) or from Integrated DNA Technologies (IDT). Using Fluidigm's More Assays software, we are able to design primers pairs for multiple loci on a chromosome for which only a single 8-base LNA (locked nucleic acid) probe needs to be used. The use of the LNA probes allows the quantitation of molecules from multiple loci on one chromosome with a single probe. A simple PCR step of limited number of cycles using tagged locus-specific primers enable the use of only a single pair of primers with the LNA probe in the digital array quantitation so that the high background problem associated with the use of multiple primers and probes in the multiplex TaqMan can be avoided.

### Assay Design

The primers were designed using the “Assay Generator” software developed at Fluidigm Corporation (South San Francisco, Calif.). Probe 19 (CTCCAGCC) was used for chromosome 18 loci and probe 20 (CCAGCCAG) for chromosome 21 loci. Given the highly fragmented status of the fetal DNA in maternal plasma, only the amplicons less than 80 bp were selected and examined using the UCSC Genome Browser (http://genome.ucse.edu/) to ensure that they did not contain known SNPs in the primer or probe sequences and were not in the known repetitive sequence regions. FAM-labeled probe 19 was obtained from Roche Diagnostics Corporation (Indianapolis, Ind.). Cy5-labeled probe 20 and all primers were synthesized by Integrated DNA Technologies (Coralville, Iowa).

### Assay Validation

Primer pairs were tested on Fluidigm's M48 dynamic array chips. Each 10-nl reaction contained 1× TaqMan GTXpress Master Mix (Applied Biosystems, Foster City, Calif.), 200 nM primers, 100 nM probes, 50-200 copies of human genomic DNA. The chips were thermocycled on the BioMark system (http://www.fluidigm.com/products/biomark-main.html) and the conditions were 95° C., 10-minute hot start and 40 cycles of 95° C. for 15 seconds and 60° C. for 1 minute.

A small number of amplicons cross-hybridized with the probe from a different chromosome and were therefore eliminated. Primer dimer tests were run on M96 dynamic array chips. The conditions were similar except that no probe or DNA was included in the reaction and 1 nM primers and 1× Eva Green dye were used. Primers which generated primer-dimers were removed. In the end, 8 loci for chromosome 18 and 8 loci from chromosome 21 were chosen. Their sequences were shown in Table 5. A total of 4 different tagged primers were used are also shown in the table, and include:

(a) a single common 5′ tag for all chromosome 18 forward primers;

(b) a single 5′ tag for all chromosome 18 reverse primers;

(c) a single common 5′ tag for all chromosome 21 forward primers; and

(d) a single 5′ tag for all chromosome 21 reverse primers.

### Tagging the Target Loci

A multiplex PCR reaction containing all 16 pairs of tagged primers was performed on 16 plasma DNA samples on a GeneAmp PCR system (Applied Biosystems, Foster City, Calif.). Each 50 nl reaction contained 1× TaqMan PreAmp master mix (Applied Biosystems, Foster City, Calif.), 100 nM each of 32 primers, 2.4 ng/nl tRNA (Sigma, St. Louis, Mo.) and plasma DNA from 5 ml of plasma. Thermocycling conditions were 95° C., 10-minute hot start and 12 cycles of 95° C. for 15 seconds and 60° C. for 6 minutes. The products were diluted prior to the copy number analysis on the HD-digital array based on their initial concentrations,

### Determining the 21/18 Ratio Using the HD-Digital Array (48.770 Digital Array)

HD-digital arrays were used to quantitate chromosome 18 and 21 molecules at the 8 loci on each chromosome using two pairs of tag primers and two UPL probes. One chip was routinely used for each sample. The products from the multiplex PCR reaction were mixed with other reagents so that the final reaction mix contained 1× TaqMan GTXpress master mix, 250 nM primers, 100 nM probes, 2× sample loading reagent (Fluidigm, South San Francisco, Calif.), and 400 to 800 molecules for each chromosome. The reaction mix was uniformly partitioned into the 770 reaction chambers of each panel and the HD-digital array was thermocycled on the BioMark system. Thermocycling conditions included a 95° C., 1 minute hotstart followed by 50 cycles of 3-step PCR; 15 seconds at 95° C. for denaturing, 5 seconds at 70° C. and 1 minute at 60° C. for annealing and extension. Chromosome 18 and 21 molecules were amplified by the two pairs of tag primers, respectively. Fluorescent signals were recorded at the end of each PCR cycle. FAM signal could be detected in any chamber containing one or more chromosome 18 molecules while Cy5 signal indicated the presence of at least one chromosome 21 molecule. After the reaction was completed, Digital PCR Analysis software (Fluidigm, South San Francisco, Calif.) was used to process the data and count the number of both FAM-positive chambers and Cy5-positive chambers in each panel. The ratio of the number of chromosome 21 molecules to the number of chromosome 18 molecules was calculated as described, as well as the 95% confidence interval.

### Results:

To detect two trisomies (18 and 210 simultaneously, we quantitated 8 loci on each of chromosomes 18 and 21 and calculated their ratio. A FAM-labeled probe was used for the 8 loci on chromosome 18 and Cy5-labeled probe for the 8 loci on chromosome 21. In a blind test, we analyzed a total of 14 pregnancy plasma samples on HD digital array chips (FIG. 7B). We correctly identified 2 trisomy 18 and 2 trisomy 21 samples. Two false positives were also found in the normal samples. Since different loci are fragmented differently, for a given set of limited number of loci, the 21/18 ratio will fluctuate and sometimes the amplitude of the variation can be greater than the difference between the ratio of trisomy plasma and a normal plasma. By using loci on each chromosome we will be able to smooth out the fluctuation and improve our results.

TABLE 5

| | |
|---|---|
| Forward | Reverse |

Chromosome 18 primers

| | |
|---|---|
| tcaattccaggtgtgcgaaaAAGAGAAGATGATACCGATTTGC | tggacgagcaacagcactataaaCCCACCATGCCAACTGAAAA |
| tcaattccaggtgtgcgaaaCCCACAATCCACCCCAGATT | tggacgagcaacagcactataaaGGAATGGGCTCTACAGAAGCT |
| tcaattccaggtgtgcgaaaCTGGCAAGATGCTGGCTTAC | tggacgagcaacagcactataaaTGTTGCTAGGATCAAGGCTGATA |
| tcaattccaggtgtgcgaaaAGAGGGTGGTCTCTCTGACA | tggacgagcaacagcactataaaTGCTTCTGGCGGATAGTGTC |
| tcaattccaggtgtgcgaaaGCGCCAAGTCCTGCATATTT | tggacgagcaacagcactataaaAACAACCCTCCAGGGTACCA |
| tcaattccaggtgtgcgaaaAGTACCCACCCCAAGAATCG | tggacgagcaacagcactataaaCCTGGCTCAGGCATGATGAA |
| tcaattccaggtgtgcgaaaACATCCACACAGCACAGGAG | tggacgagcaacagcactataaaTGTGCTTGATGGCTCTGCAT |
| tcaattccaggtgtgcgaaaCCTGCAGGCTACTGTCCATT | tggacgagcaacagcactataaaGACAGGGTGGCTCTGTATCC |

Chromosome 21 primers

| | |
|---|---|
| tgcaacgagttagtggaacagaatGGTTGAACCACCAACCACAT | acagcacaactcgcaattgaaTGGCAATGGAATTCTCTTGGA |
| tgcaacgagttagtggaacagaatCCGGACACATGCCTTCTGG | acagcacaactcgcaattgaaCCGTCTGTGCTGGTTATTGG |
| tgcaacgagttagtggaacagaatTCAGAGAACATCCGCTTCCT | acagcacaactcgcaattgaaTCCATGGACGCTTCCTTCTT |
| tgcaacgagttagtggaacagaatAGACCGAGGCTGCCTTCT | acagcacaactcgcaattgaaTTGCCCTCCGCTCTCACT |
| tgcaacgagttagtggaacagaatTGGCCAATCAGAAGAGTCAGG | acagcacaactcgcaattgaaAGCTGCTCAGAACACCTAGA |
| tgcaacgagttagtggaacagaatGCTGCTCCTGCTTGTCTTTC | acagcacaactcgcaattgaaGTGTGCAGTCCCAGGTCTAC |
| tgcaacgagttagtggaacagaatGACCTTCCTCACCCCATAGC | acagcacaactcgcaattgaaTCAAAGCTGCTGTGCTGAAT |
| tgcaacgagttagtggaacagaatAGGCTCTCCCCTAGACAAGT | acagcacaactcgcaattgaaACTGTCTGCAGCACATAGCA |

Tag primers
Chromosome 18

| | |
|---|---|
| TCAATTCCAGGTGTGCGAAA | TGGACGAGCAACAGCACTATAAA |

Chromosome 21

| | |
|---|---|
| TGCAACGAGTTAGTGGAACAGAAT | ACAGCACAACTCGCAATTGAA |

### Summary

We have developed a multiplexed approach to detect possible aneuploidies in maternal DNA samples. The approach can tag as many loci as needed with the same sequences that will be used for primer-annealing in the next step, so all the amplicons for a specific chromosome can be detected with a single pair of primers and a single specific probe. Although in this study, we used LNA probes as the detectors for the amplicons from different chromosomes after multiplexed PCR, this approach can be expanded to any other detection or amplification scheme, including, not restricted to the use of fluorescently labeled probes (such as TaqMan), intercalating dyes (such as SYBR Green), as well as ligation-based approaches to amplification.

An additional, but equally important, aspect of the multiplexing approach is that it enables us to overcome the limitations associated with the limited amount of, and low concentrations of, DNA extracted from plasma, which has been previously reported to be as low as 1,000 genome equivalents per ml of plasma (reduces sampling error). A further increase in multiplexing density will only serve to further reduce the sampling noise.

### Example 9

### Non-Invasive Method to Measure the Abundance of Chromosome 21-Located miRNA in Plasma to Determine Fetal Aneuploidy

This method detects miRNA derived from plasma, serum or other body fluids as a means to infer fetal aneuploidy. The method utilizes the properties that (i) chromosome 21 located miRNA are elevated in the plasma of mothers bearing trisomy 21 fetuses and (ii) the concentration of miRNA analytes are robust biomarkers of trisomy or other aneuploid states. Important aspects of this method are that the connection between fetal aneuploidy and miRNA abundance in plasma has not been made, miRNAs are surprisingly stable nucleic acid analytes present in many body fluids including serum and plasma, and a non-invasive method to quantitate miRNA analytes has marked advantages over the use of DNA. Plasma derived miRNA-155 is the recommended target for quantitative PCR analysis using Fluidigm Digital Array™ IFCs

### Problem:

Non-invasive DNA-based methodologies for detecting fetal aneuploidy are difficult to perform. Reasons for this include: (i) only relatively low amounts (−5%) of total circulating DNA is fetal in origin, (ii) fetal DNA is randomly cleaved, presumably at nucleosome accessible sites such that any one loci consists of a population of different length sequences and (iii) naked circulating DNA is rapidly degraded and consequently unstable. A solution to DNA length, abundance and lability issues is to examine plasma abundance of microRNAs located on chromosome 21.

The instant method utilizes the abundance of chromosome 21-located miRNA in plasma as a method to determine fetal trisomy 21.

Plasma abundance of miRNA located on chromosome 21 as indicators of fetal trisomy 21 has not been investigated. Overexpression of the chromosome 21 located microRNA (mir155) downregulates a number of important protein targets, including the master regulating transcription factor methyl-CpG-binding protein (MeCP2). Moreover, decreased MeCP2 contributes to the Down Syndrome phenotype in humans and mice.

MicroRNA are becoming recognized as remarkably stable and abundant entities that are easily detectable in serum and plasma. An assay that detects elevated levels of chromosome 21 located miRNA in the plasma of mothers bearing trisomy 21 aneuploid fetuses versus normal diploid fetuses has significant value. The method of the invention employs qPCR, digital-PCR, ligation-mediated PCR or ligation chain reaction to quantify microRNAs derived from plasma, serum or other body fluids as a means to screen for fetal aneuploidy. DNA targeted by small RNAs may display increased resistance to in vivo nucleases and may also be a preferred target for these assays.

TaqMan-type reagents have been used to detect miRNA sourced from female (pregnancy) and male (prostate cancer) serum. Data from those experiments strongly confirm that those miRNA are present as high copy number, discrete 22-mer length products.

The chromosome abnormality in Down syndrome (DS) is a consequence of a triplication (extra copy) of an entire copy or a portion of human chromosome 21 (Hsa21). However, it is unclear how this aneuploidy/copy number variation causes the DS phenotype.

Mature miRNA are ˜22 nucleotide length species found in varying amounts in tissue-dependent manner. miRNA decrease gene expression by (i) inhibiting translation and/or (ii) promoting messenger RNA (mRNA) degradation by base-pairing to complementary sequences within protein-coding and/or 3′ untranslated regions of mRNA transcripts.

miRNA are remarkably stable as discrete-length, ˜22-mers in human body fluids and tissues. This fact is anti-intuitive and consequently, not widely known.

There are 5 known miRNAs located on Hsa21. miRNA in situ hybridization experiments demonstrate that all five Hsa21-derived miRNAs are over-expressed in brain and heart specimens from individuals with DS. One of these miRNAs is known as miRNA-155. When miRNA-155 is upreguated it binds to the master regulating transcription factor methyl-CpG-binding protein (MeCP2). This is important because mutations in MeCP2 contribute to the DS phenotype. MeCP2 is under-expressed in human fetal and adult DS brain specimens. Independent of the above lines of evidence, placenta is the main source of circulating fetally-derived nucleic acids. A recent study (Luo et al. 2009, Biology of Reproduction 81, 711-729) that screened for all small RNA species in first trimester placenta, whole villi and full term placenta did not discover miRNA155 in the blood of women bearing non-trisomy 21 fetuses. This indicates the background level of this miRNA is very low in normal patients. As a consequence, this is expected to aid interpretation of quantitative data.

### Example 10

### Nexgen Sequencing Detection of Fetal Aneuploidy with Amplicon Tagging

The method of fetal aneuploidy detection by next generation sequencers as published by both Quake and Lo utilize the standard sequencing prep method (fragmentation, ligation of adapters and emulsion or bridge PCR). Although this gives broad coverage of each chromosome, it uses the sequencing space very inefficiently. Amplicon tagging with barcoding would be much more efficient because:

(1) Barcoding allows sample multiplexing.

(2) Amplicon tagging skips the fragmentation and ligation steps.

(3) The need for GC bias correction is avoided.

(4) Depth issues associated with unequal sequence coverage are avoided with well designed loci.

(5) The preparative steps can be done in the Access Array™ system, very much simplifying the workflow.

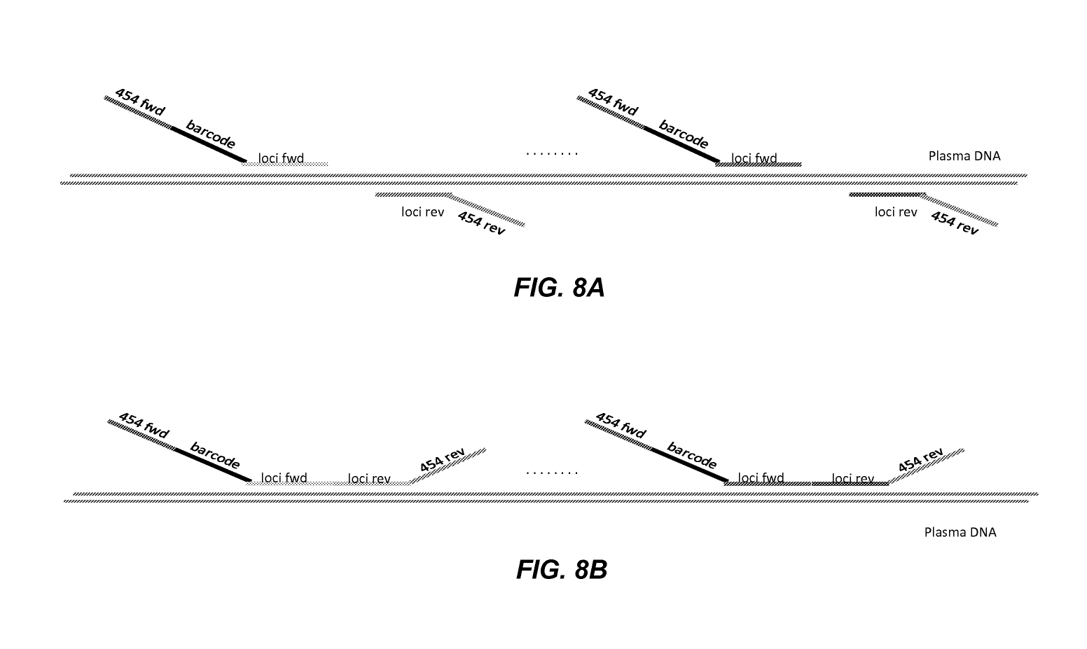

Two of the possible methods to do this are by pre-PCR (see FIG. 8A) and by Ligation (see FIG. 8B), both using primers tailed with the barcodes common sequencing tails. This multiplex reaction is performed and then run on the sequencer. They both create reactions products that can be identified and counted to make the determination of aneuploidy.

### Example 11

### SNP Detection Via Target-Specific Ligation Followed by Stuffer-Based Tm Selection

### Problem Statement:

SNP genotyping can be performed using microarrays, TaqMan, mass spectroscopy and DNA melting differences (Tm). Fluidigm has developed TaqMan genotyping for Dynamic Array™ integrated fluidic circuits. However, TaqMan assays are expensive, requiring proprietary reagents and are limited in their ability to distinguish low frequency rare mutations within a large background of normal genotypes.

There is significant demand to not only decrease genotyping costs using Tm but also detect rare SNP mutations within a large population of wild-type genetic material (i.e. needle-in-a-haystack type applications).

Of the assays described above, Tm is the cheapest and arguably given sufficient Tm difference between amplicons, the simplest and most direct measure. However, Fluidigm's (and other companies) ability to measure small Tm differences are constrained by both the biochemical assays and equipment technical resolution. The ability to increase the Tm differences between SNPs whilst retaining high SNP discrimination ability would provide significant technical and biological value.

The current method employs the highly selective ligase detection assay to hybridize Tm discriminating oligonucleotides to a SNP of interest. In the preferred embodiment, one of the SNP-targeting primers contains a high Tm permissive G:C rich stuffer domain. The second SNP-targeting primer contains a low Tm permissive A:T rich stuffer domain. Cycles of target denaturation followed by ligation, in the absence of dNTPs, are repeated, enriching for the ligated SNP target. Highly selective ligation occurs if the SNP-targeting single nucleotide flap is cleaved to reveal a 5′ phosphate group using the FLAP endonuclease of a non-hotstart, heat-tolerant polymerase such as native (non-hotstart) Thermus aquaticus DNA polymerase 1.

Following target specific SNP enrichment, typical Tm asymmetric PCR is performed. Comparing the Tm differences between the G:C and A:T rich stuffer domains is thermodynamically calculated to widen a small SNP Tm difference of 1° C. by an order of magnitude (10° C.), well within the existing discriminative ability of most/all SNP-Tm capable instruments.

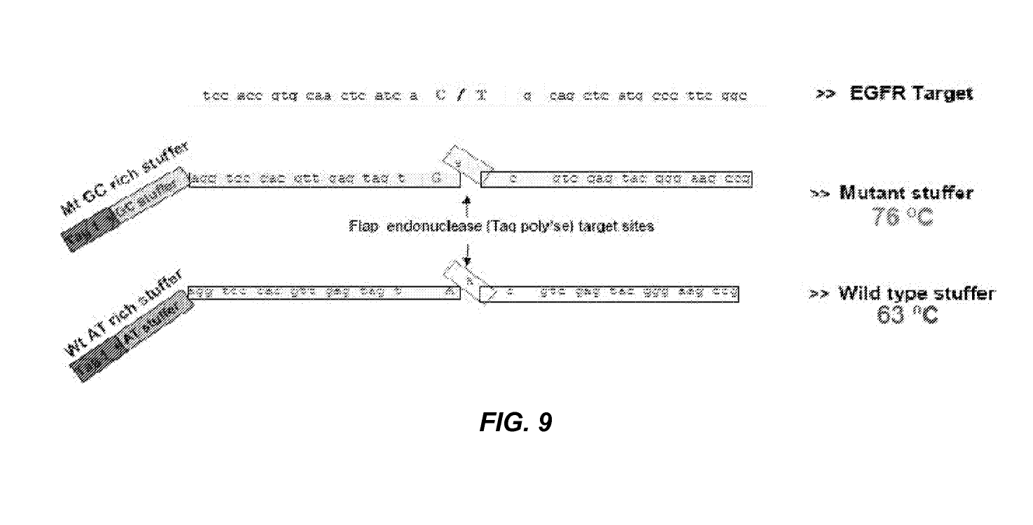

A description of the Tm-enhancing-stuffer ligation procedure is given i FIG. 9. As an example of the utility of the assay detection of the clinically-relevant EGFR T790M mutation, responsible for mediating resistance to anti-cancer medication is shown. The general procedure entails:

(1) Ligation PreAmp with Tm distinguishing stuffers (akin to a standard preamplification);

(2) Taq FN activity cleaves flap, revealing a 5′ phosphate group, permitting ligation; cycle 50 times;

(3) Asymmetric PCR amplification on a DID-type chip; and

(4) Compare amplicon Tm difference between Mt (GC-rich stuffer) versus Wt (GC-poor stuffer).

### Example 12

### Pre-Amplification and Amplification Methods Based on Target-Specific Ligation Via LCR/LDR (Ligase Chain and Ligase Detection Reaction) Followed by PCR

Fetal plasma DNA is relatively scarce and typically nucleosome-length compared to maternal DNA. However, the ability to differentiate between these DNA species is non-trivial. The ability to differentiate between fetal and maternal DNA is of significant diagnostic value.

A method for selectively detecting detecting low abundance, low MW fetal DNAs in a large background of maternal DNA is a ligation-based selection of specific plasma DNA sequences to both increase the copy number and enrich for these limited target molecules. The method permits multiple sub-sequences per target to be assayed (e.g. 10 assays for chromosome 21). These sequences can be located either far apart or in close proximity including those directly abutting each other.

### PCR:

By adding tags to the primers, groups of products (e.g. chromosome 21 target sequences) can then be detected by a common primer pair in digital PCR, and the presence of one or more chromosome 21 fragments per reaction is assessed.

Regular PCR pre-amplification of plasma DNA may result in a loss of differential fragment length information between fetal and maternal DNA. It is possible to reduce this effect by endo-/exo-nuclease cleavage (TaqMan-like) of primers that have annealed downstream of another primer. This may be enhanced by using primers with 5′ tag.

2 favored solutions include the use of multiple non-linked sandwich amplicons (each testing coincidence of outer primers) or multiple neighboring amplicons.

### Ligation:

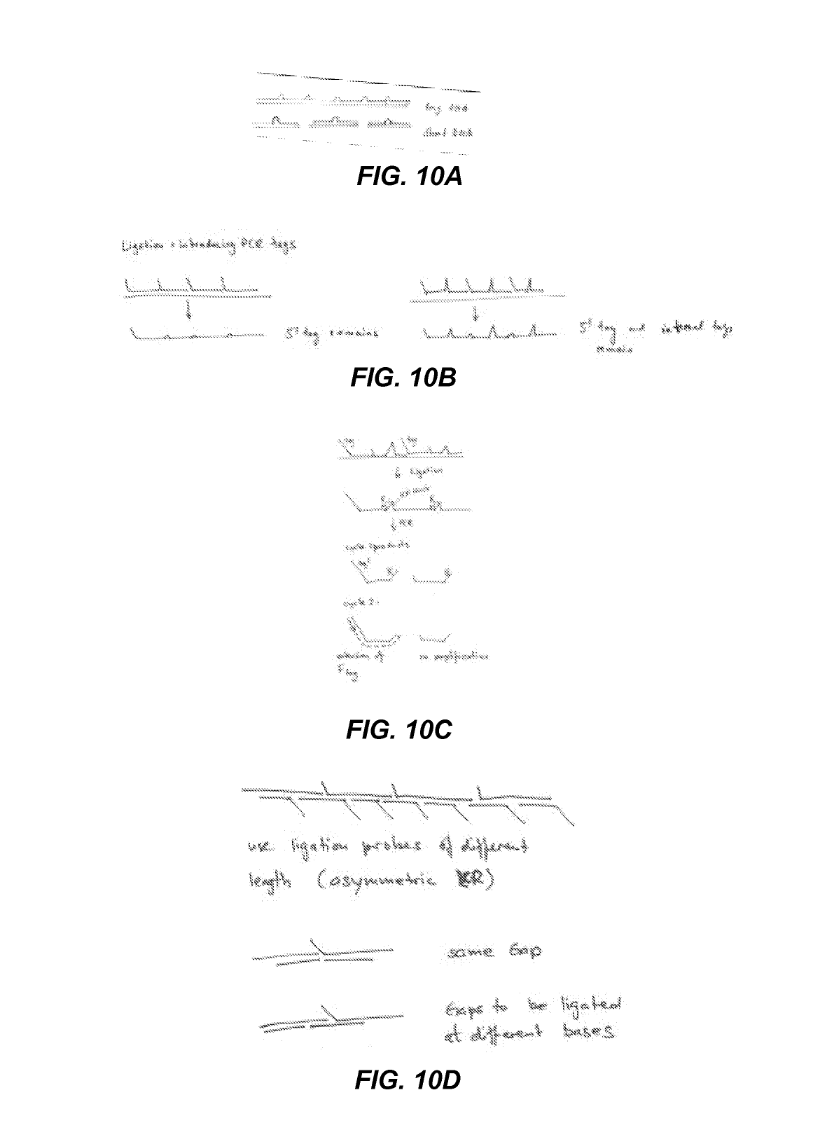

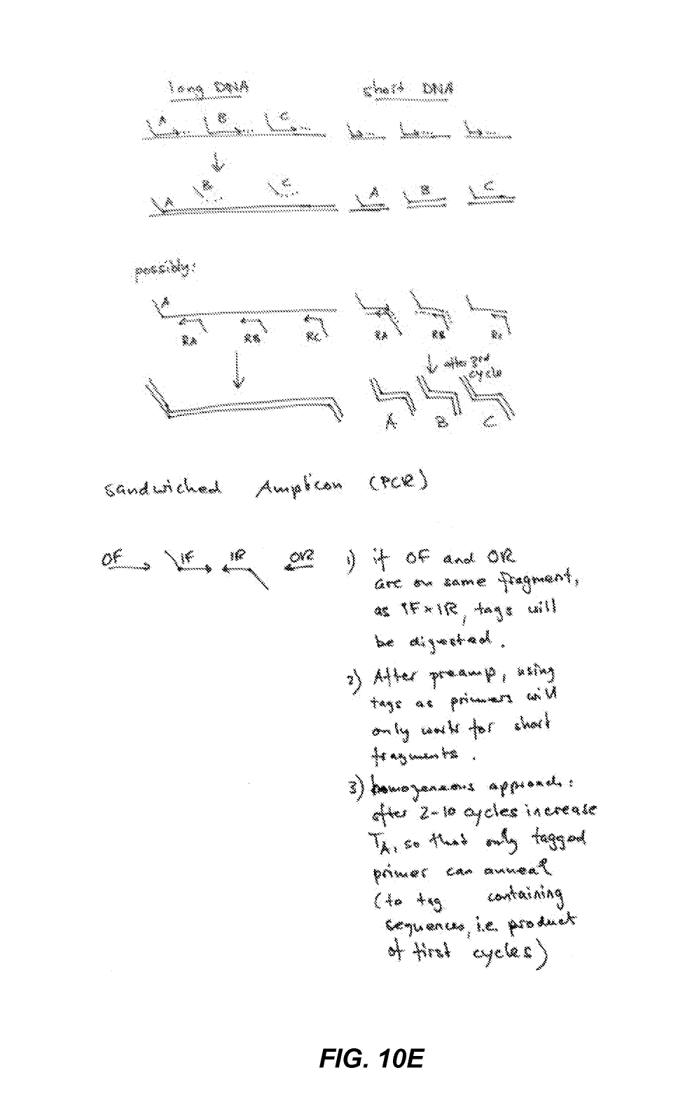

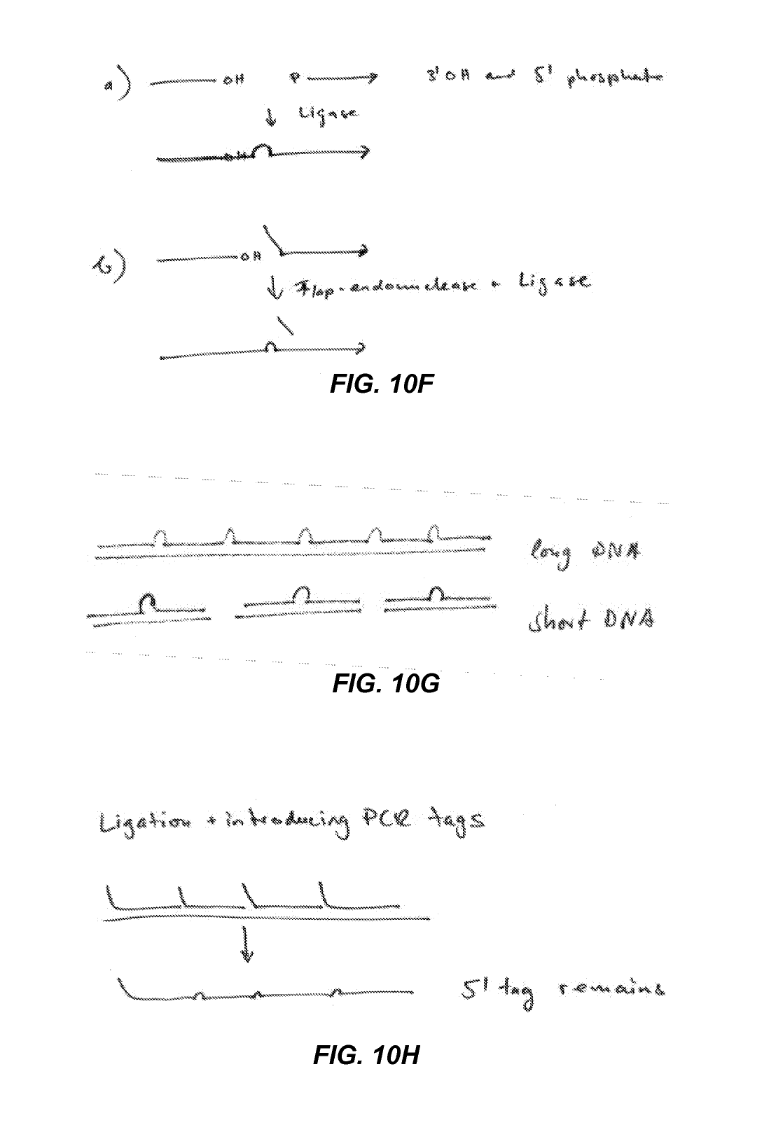

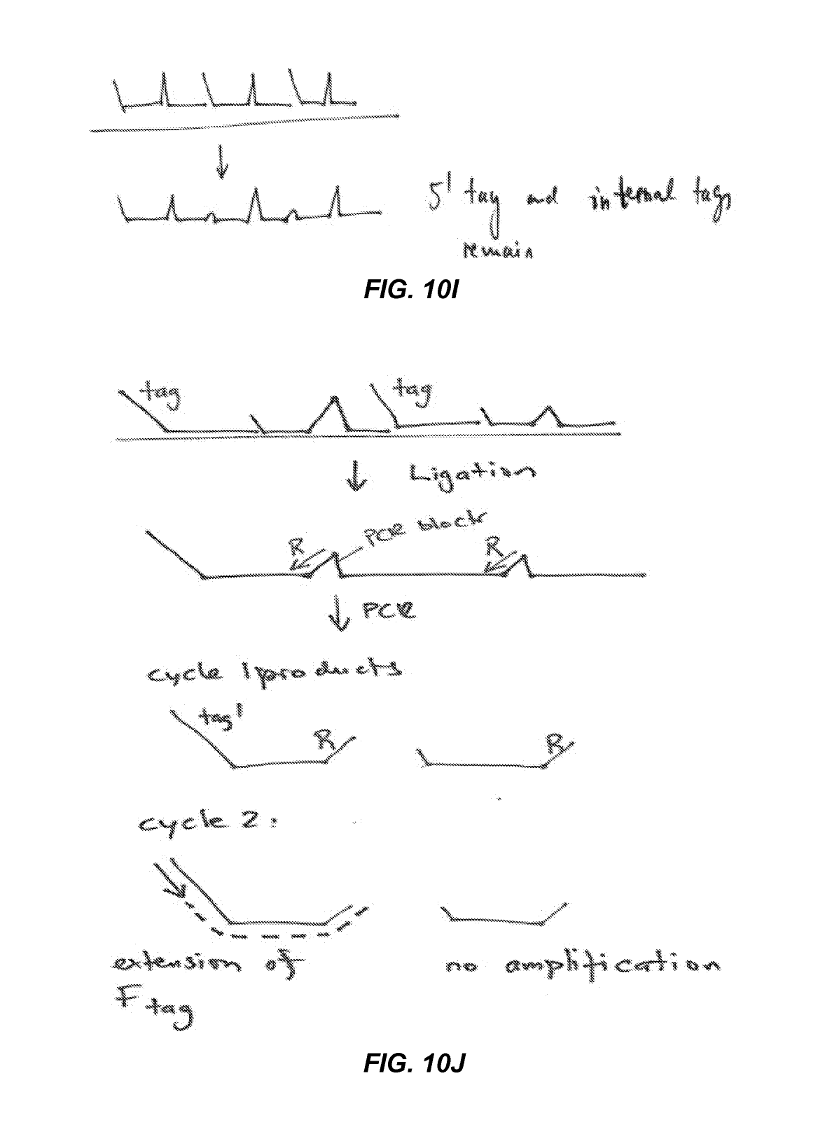

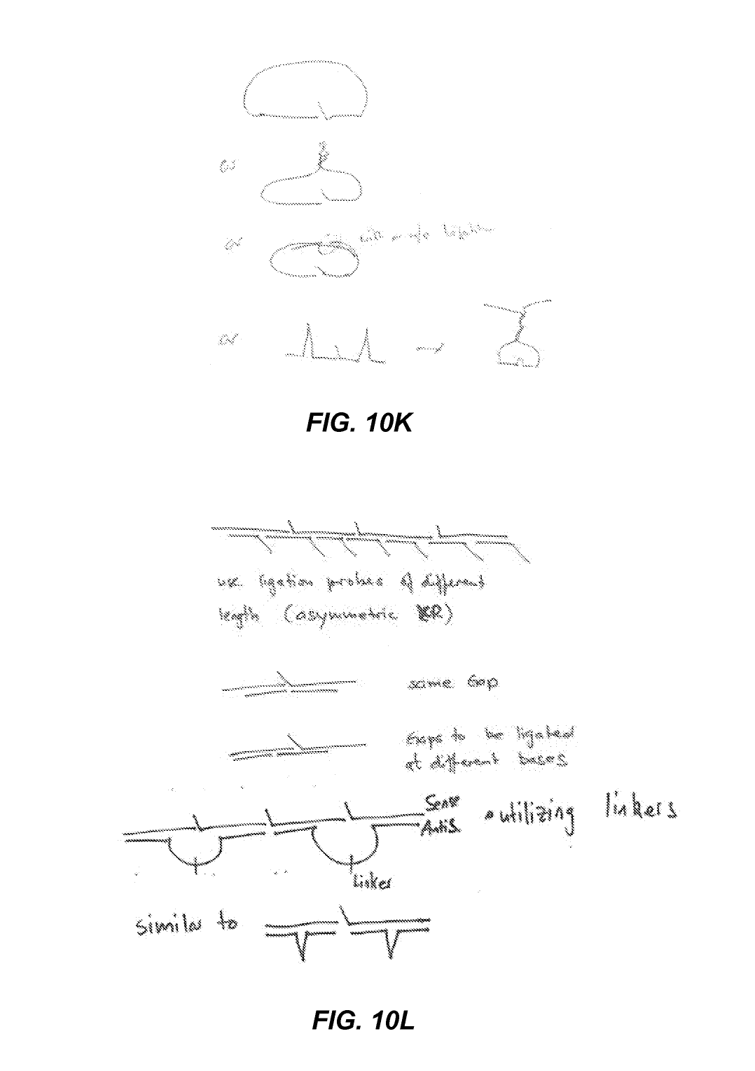

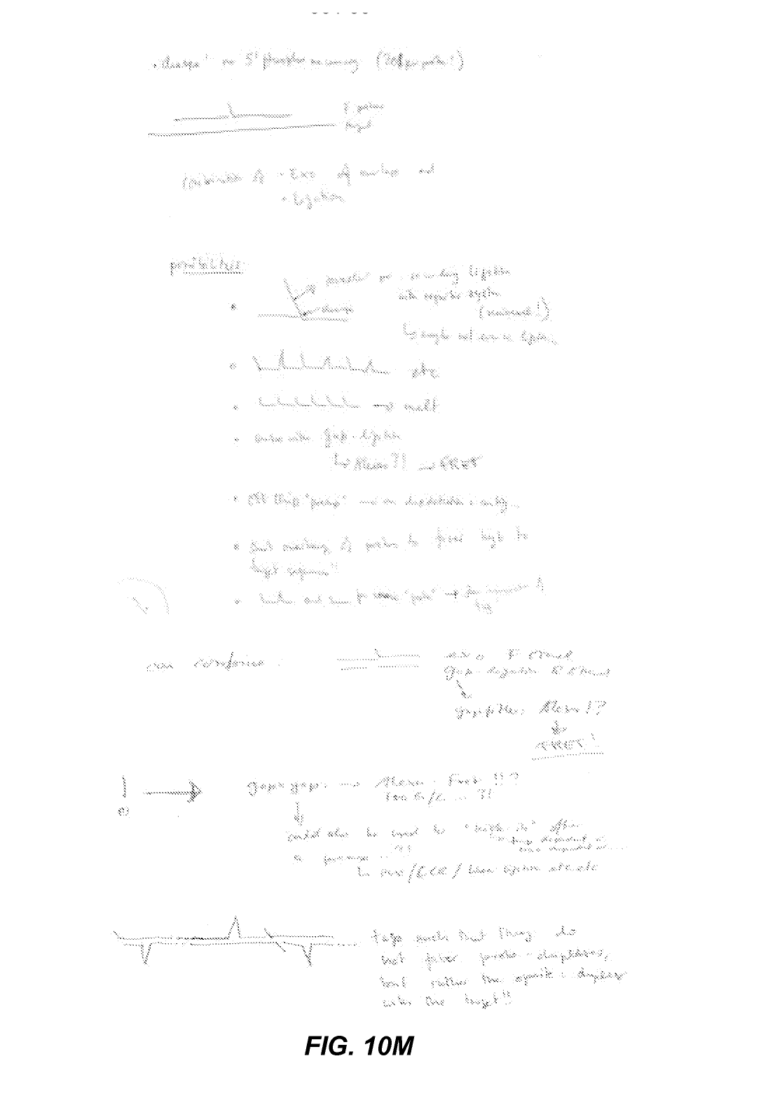

PCR target-specific amplification of plasma DNA may result in a loss of different fragment lengths for fetal and maternal DNA. However, ligation of multiple (3 or more) neighboring/consecutive probes retains DNA length information, and enriches for these products as only probes hybridizing to the same fragment are ligation competent. A long DNA fragment will yield one long product whereas the same sequence in 10 fragments can yield up to 10 shorter ligation products. Performing multiple temperature cycles with a temperature-resistant ligase permits one strand of the target fragment(s) to be linearly amplified up to 500-fold via the ligase-detection/ligase chain assays. See FIG. 10A.

It is possible to introduce tags for downstream functionalities, such as PCR. Tag/tail sequences can be appended at the 5′ end of a ligation probe (left). New: tags can be added in the middle of a ligation probe. In this fashion, both ends of the probes are available for ligation, permitting to produce a ligated chain of probes. After ligation preamplification, ligation products can be detected, e.g. in the Digital Array™ integrated fluidic circuit by PCR. See FIG. 10B.

In one embodiment, the 5′ tag of every 2^{nd} probe can be used as priming site (tag has same sequence as a PCR primer), and the inner tag of every other 2^{nd} probe will serve as binding site of a second primer (and have a linker molecule that halts downstream PCR). This permits selective amplification of the two 5′ most probes, as only the 5′ tag in the ligation product is the one of the first ligation probe (by using 5′ tag and internal tag as primers) regardless if the ligation product is long or short. See FIG. 10C.

In a second embodiment, ligation chain reaction (LCR) using 3 or more consecutive probes per strand (sense and antisense) can serve as a target-specific amplification step that retains target size information. It may also be used as single step digital (e.g. on chip) assay to determine the number of fragments (i.e. molecules containing a target sequence). Embodiments to prevent target-independent DNA ligation, blunt-end ligation: FLAP-exonuclease overhangs (tags), one or both strands' probe-pairs, GAP-ligation on one probe pair are anticipated.

Asymmetric LCR/LDR where either the sense or antisense strands are differentially targeted for preferred ligation by use of oligonucleotides that differentially hybridize in a temperature-dependent manner (e.g. enrich for 1^{st} strand product for 100 cycles, prior to switching to a lower ligation temperature prior to LCR or LDR). On bottom of figure are two simple embodiments, using chains of probes etc may be also positive. See FIG. 10C.

### Beneficial Aspects of the Present Method Include:

Preamplification of multiple targets of one “target group” with the same tags to detect the presence of one or more of the subsequences in a downstream assay, e.g. digital PCR on chip.

Preamplification with tagged primers to increase the annealing temperature after low number of cycles (2-5—10).

Application of tagged primers in PCR (especially preamplification, but also as homogeneous assay) of consecutive/neighboring/flanking sequences to enhance degradation of “inner” products (sandwich).

Chain of consecutive ligation and flap-endonuclease capable probes for use in a ligase chain or ligase detection assay.

Nucleotide tag (inserts) in probe for downstream functionalities such as serving as primer binding site.

Also use of 5′FLAP (=5′ tag) as functional site if not cleaved in ligation). Described: use as PCR primer site.

Spacer in insert-tag to block formation of long PCR products.

See exemplary PCR approaches described in FIG. 10E.

Ligation:

2 main schemes of ligation are used in this method as examples (and preferred embodiments) of invention: a) 5′-phosphate, b) overhang of one or more nucleotides (Flap) which is cleaved by a flap-endonuclease (e.g. Taq Polymerase) resulting in a ligation competent 5′-phosphate. See exemplary ligation approaches described in FIG. 10F.

One Form of the Invention is Using More than 2 Adjacent Probes for Ligation (See FIG. 10G):

In a first solution all Forward probes are tagged (e.g. with a common set-specific tag). See FIG. 10H.

Probes can also contain internal tags not complementary to the target sequence. See FIG. 10I.

5′ tag and internal tag in alternating probes, PCR of ligation product. See FIG. 10J.

### Variations/Modifications

Further variations/modifications are shown in FIG. 10K-10M. An approach using exo-nuclease resistance is shown in FIG. 10L. See Livak et al., U.S. Pat. No. 6,511,810, which is hereby incorporated by reference for its description of FLAP ligation.

### Example 13

### Ligation or PCR-Based Target-Specific Super-Plexing Using Universal Sequences and Combinatorial Tag Primers for Simultaneous Detection of Multiple Nucleic Acid Sequences

### Includes the Following Embodiments:

Direct detection of RNA without reverse transcription

Use of a single probe (preferably) or Universal Probe Library

Encoding protocol for multiple transcripts permitting DNA sequencing using a harvestable microfluidic chip.

Multiplex PCR primer/probe strategies are a convenient approach to simultaneously amplify/detect multiple amplicons. However, multiplex PCR is limited in the number of primers that can be combined, for reasons including the propensity of high concentration primers to form inter-primer complexes and identifying amplification conditions that permit robust product specificity. Essential cDNA synthesis procedures add further complexity. In-house, these intrinsic problems are heightened when considering using the Roche Universal Probe Library (UPL) where thousands of primers are combined yet only a maximum of 165 hydrolysis probes are used to separately distinguish specific amplicons. These issues constrain development of applications that are particularly advantageous using the Fluidigm Dynamic Array™ IFC.

The Ligase Detection Reaction (LDR) or PCR in combination with target-specific oligos bearing 2 Universal Preamp target sites and target-specific tag sequences to ameliorate primer interaction issues observed in multiplex PCR. Addition of Universal Preamp priming sites permits the use of only two common primers to simultaneously multiplex-preamp all ligation products. i.e. “Super-Plexing”. Superplex primers also bear 1-of-100 different tag primers on the 5′ and 3′ target-specific primers. This permits 10,000 combinatorial tag variants representing 10,000 specific RNAs or genes to be amplified using a discrete set of limited primers.

An embodiment of LDR, permitting direct oligonucleotide hybridization and subsequent ligation using an RNA template, bypassing the requirement for a cDNA synthesis step is described. This is essentially the same as for DNA template.

In a further embodiment, probe (preferably a high affinity single sequence or possibly UPL) binding sequences are added to the sequence of an amplifying primer. Extension by the reverse primer hydrolyses bound probe. This approach simplifies assay design and normalizes UPL probe sequence context ensuring all assays in a single column of a Dynamic Array™ integrated fluidic circuit are capable of binding the same probe.

### Solution:

(1) Employ the LDR to specifically ligate 2 Universal Super plex sequences to all targets of interest.

(2) Simultaneously append combinatorial tags permitting, for example, 100 primers to represent 10,000 possible amplicons.

An embodiment of the method uses the Ligase Detection Reaction (LDR) in combination with target-specific oligonucleotides bearing:
(i) Two Universal sequences permitting Super-plexed amplification, preferably in a generic non-specialized “preamp” buffer, and
(ii) Two separate tag sequences.

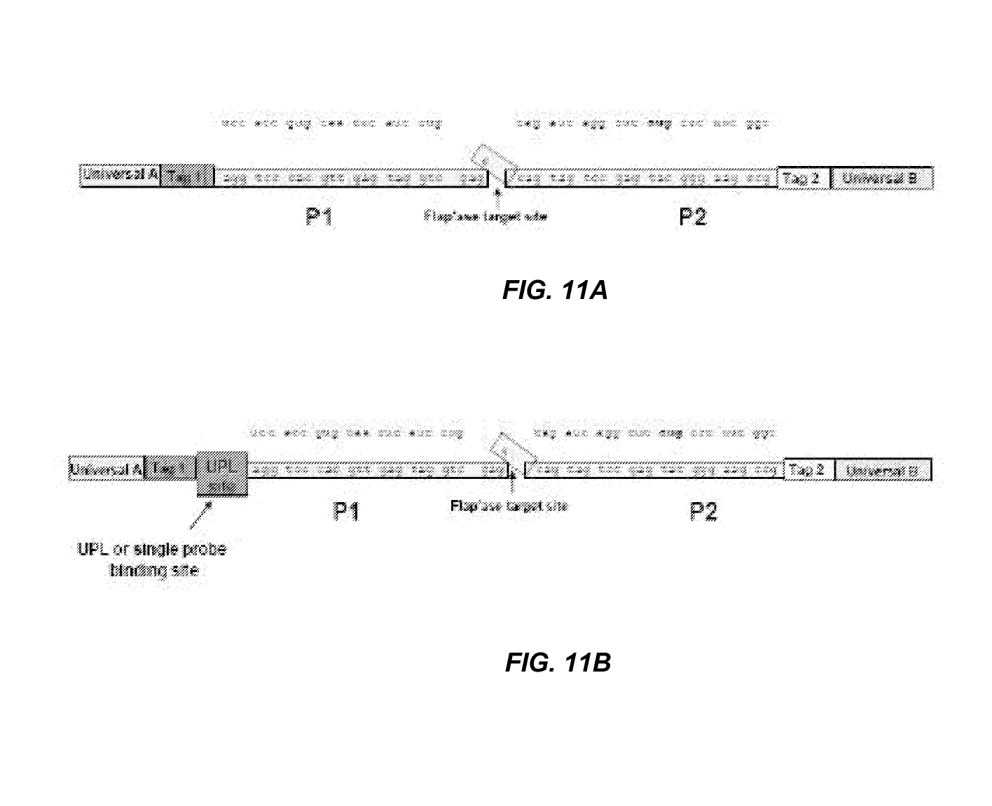

The Universal Preamp sequences permit simultaneously multiplex-preamp “Super-plexing” of all ligation products using only 2 primers. This overcomes traditional multiplex primer:primer interactions limitations to PCR. Target-specific oligos also bear one of 100 different tag primers on the 5′ primer and 1-of-100 different tag primers on the 3′ primer. Different paired tags permit coherent generation of 10,000 combinatorial variants, where each variant represents 1-of-10,000 specific RNAs or genes amplicons. See FIG. 11A.

An embodiment of this approach permits oligonucleotide ligation via direct hybridization to an RNA target without the need for a cDNA synthesis step.

A Super-plexed LDR methodology bypassing the requirement for a cDNA synthesis step is described: RNA detection assays almost invariably require converting an RNA template to either single or ds cDNA. intermediate prior to subsequent amplification. cDNA synthesis i.e. “reverse transcription” is the crucial first step for RT-PCR and microarray sample preparation steps (Eberwine reactions) and the majority of RNA sequencing and cloning methods. This is considered the most user and reagent variable consideration when examining RNA expression. Although cDNA synthesis is a critical step in these methods, it remains expensive, requires specialized enzymes, carefully prepared reagents, attention to primer/enzyme read-through of structured RNA and avoidance of RNase's and metal ion-mediated degradation. A solution to this issue is to directly hybridize Universal Superplex tag primers to the RNA and ligate. Repetitive thermal cycling is not necessary. This approach combines high target specificity and decreased biochemical complexity for specifically amplifying small RNA species such as microRNAs. See FIG. 11A.

In a preferred embodiment, a single optimal probe or plausibly 8-mer UPL binding sequences are directly added to the sequence of a single primer: The UPL system permits a set of 165 locked nucleic acid hydrolysis probes of 8 or 9-mers to robustly hybridize to a large variety of sequences. However, use of this system compromises PCR application design because the limited numbers of available probes must firstly exist in the desired PCR product and secondly the probe binding site must be readily accessible to that sequence. These requirements vary on an amplicon-by-amplicon basis. Adding a single optimal probe, or one of the 165 probe binding sequences directly to the forward extension primer guarantees that all amplicons in the single column of a Dynamic Array™ IFC contain a single probe sequence in exactly the same sequence context. A single optimal probe or UPL probes are added separately to other PCR components. Probe hydrolysis occurs when the antisense primer extends to displace the probe. This arrangement simplifies assay design, sample tracking and software fluorescence deconvolution. Simplified PCR primer/UPL probe multiplexing schemes enhance both assay performance and reproducibility while highlighting the advantages of the Fluidigm Dynamic Array™ IFC platform. See FIG. 11B.

A further embodiment of this approach for increasing the number of assays that can be conducted on a Dynamic Array™ IFC for sequencing applications follows: A critical change is that after harvesting from the chip, additional, downstream reactions can be carried out using other methods (e.g. sequencing).

The method involves four steps:

(1) Design primers for the amplicons of interest to contain amplicon specific sequences and tag sequences.

(2) A multiplex pre-amplification or encoding step in which amplicons are amplified specific to the designed primer sequences.

(3) A second amplification step in which tagged primers are amplified using the tag sequences in a microfluidic device.

(4) A harvesting step in which a portion of the amplified product is harvested from the microfluidic device.

Step (1)(a) design forward primers for (M*N) amplicons to contain an amplicon specific region and a 5′ tag selected from a set of M 5′ tags. This will produce N sets of oligos for each tag sequence.

Step (1)(b) design reverse primers to contain an amplicon specific region and a 3′ tag selected from a set of N 3′ tags. N 3′ tags should be chosen so that each amplicon will contain a unique pair of M and N tags.

Step (1)(c) design M primers that contain only one each of the M tag sequences. Design N primers that contain only one each of the N tag sequences.

Primers designed in Steps (a), (b) and (c) should be designed to have similar Tm values at low concentrations (they will be present at low nM concentrations in the final mixtures).

Step (2)(a) Prepare a mixture containing all primers designed in step 1. Add mixture to sample and amplify for a small number of cycles within which amplification should be linear rather than exponential.

Step (2)(b) Partition the sample into M partitions. Add one of the M 5′ tag primers (1)(c) to each partition. Add each of the M sample/5′ primer partitions to M sample inlets on the microfluidic device. Add one each of the N 3′ tag primers (1)(c) to N reagent inlets on the microfluidic device.

In approach valuable for all embodiments, oligonucleotide generation can be massively simplified Agilent Technologies' Oligonucleotide Library Synthesis for parallel synthesis of oligonucleotides (up to 55,000 unique oligonucleotides with length of 200-mer). After chemical removal from microarray surface, oligos are lyophilized. The lyophilized material contains the pool of target specific oligos bearing common universal sequences.

In an embodiment, the linear amplification ligase detection assay rather than exponential ligase chain reaction is utilized. In a further embodiment, the method employs a single flap-bearing primer rather than 2 flap-bearing primers. In additional embodiments universal Super-plexing sequences and combinatorial tagging with or without LDR to achieve simplified PCR is used. And, the addition of a single probe binding sequence (preferably) or a separate UPL binding sequences directly to primers to enhanced multiplexing capability is provided for.

### Example 14

### Use of Common Sequence Motifs (with Pre-Amplification and Digital PCR) for the Enhanced Multiplexing of Targets for the Detection and Quantification of Fetal Aneuploidy

### Problem Statement:

For fetal aneuploidy detection by using digital PCR (including microfluidic dPCR and emulsion PCR) it is desirable to pre-amplify the number of target molecules without bias in order to achieve precise quantification. This includes the amplification of multiple different targets (e.g. DSCR (Down Syndrome Critical Region), chromosome 18, etc) and multiple loci per target. It is demonstrated that PCR based multiplex PCR can actually be reproducible enough to meet this requirement for different loci.

For the detection of different loci per chromosome assays with a common 8 nucleotide sequence between the primers, which is used as target for a dual-labeled LNA hydrolysis probe have been used. In essence, this is the use of detecting multiple targets with a shared motif by a detection probe that enables detecting said motif

### Universal Motif

The selection of a motif for the detection of multiple sequences after pre-amplification for the application of NIPD of fetal aneuploidy can be influenced by a number of considerations.

Length:

The motif should be sufficiently long to make specific detection possible (>4 nucleotides, preferably >6 or 8). But it should be sufficiently short to allow the design of a sufficiently large number of assays (≧10, or >19 or >50) (e.g. when targeting the Down Syndrome Critical Region (DSCR) on chromosome 21, which has (˜1.5 to) 5 million base pairs) a random 5-mer can be found on average ˜10′000 times, a 6-mer 2500 times, a 7-mer 600 times, an 8-mer 150 times a 9-mer 40 times, a 10-mer 10 times etc.). In longer targets, e.g. chromosome 18 (spans about 76 million base pairs) would have 30 times more targets for a given n-mer than the DSCR.

Sequence:

The sequence can be chosen such that it has a higher number of possible targets available in the range of target sequences (e.g. DSCR). This probably applies to most Universal Probe Library sequences (Roche/Exiqon).

Alternatively, the sequence may offer a certain benefit for detection, such as for example GC content, symmetry (which can be problematic, e.g., LNA probes could form dimers), etc.

Another bioinformatic way to chose the motif is to count the number of each possible or eligible e.g. 8-mer in the target sequence and choose a motif that has sufficiently many copies in the target (e.g. DSCR).

TABLE 6
A suitable motifs

| | 3′-end |
|---|---|
| motif | primers |
| CAGCTGTG | -CA |
| CAACCTTG | -CAA |
| GCAATTGG | -CAA |
| CAACCGTG | -CAA and |
| | -CAC |
| TAGACCAT | |
| NNGTCANN | -GTCA |
| GCCAGCAG | -CANCA |
| GCCACCAG | -CANCA |

TABLE 7

| | | |
|---|---|---|
| 1 | 4 | 2500000 |
| 2 | 16 | 625000 |
| 3 | 64 | 156250 |
| 4 | 256 | 39063 |
| 5 | 1024 | 9766 |
| 6 | 4096 | 2441 |
| 7 | 16384 | 610 |
| 8 | 65536 | 153 |
| 9 | 262144 | 38 |
| 10 | 1048576 | 10 |
| 11 | 4194304 | 2 |
| 12 | 16777216 | 1 |
| 13 | 67108864 | 0 |

### Alternative Detection Methods:

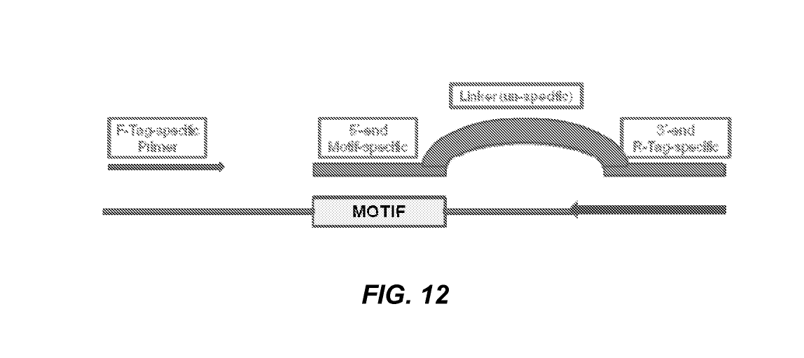

The previously used detection method for multiple assays with the same probe have been either tag-specific (probes detecting the presence of the tag in a PCR product) or motif-specific LNA modified probes. Other modification may be employed to detect the shared sequence motif. In this method, a probe is used that binds a) to one of the tags of a product and b) to a common motif for all products that are to be detected by the same probe. In this way, the probe can be quite long to promote probe binding to the PCR product (sequence to hybridize to the tag) and also confer increased specificity compared to tag only detection (by linking detection with hybridization to the target-motif). See FIG. 12.

### Target Selection and Design Workflow

Primer Design for Multiplexing:

Multiplexing will profit if one designs it to use primers with the same 3′-end sequence (reduced complexity of 3′ ends in pre-amplification). This can be exactly the same sequence or a number of 3′-end. The length of this 3′-end can be 1, 2, 3 or more nucleotides and is preferably uninterrupted, but not necessarily (e.g. —CNAA is a possible 3′-end with N being any base).

Also, certain bases are more stringent towards a perfect match (C, A) and should be favored on the 3′-end of the primer. Thus, the use of e.g. −CAA as 3′-end for all (or most) primers in a multiplex will lead to a reduced formation of unspecific products, 1) because of the reduced complexity of 3′-ends, and 2) because of the high stringency of the 3′-end.

Furthermore, for multiplexed pre-amplification it may be of benefit to pre-define the target sequence adjacent to the 3′-end of the primers.

Workflow

Sample: whole blood, serum or plasma from a pregnant woman: When performed with suppression or other enrichment strategies for short DNA fragments (physical or as part of enzymatic reactions or distinguishing reaction products from short vs. long), it is in principle possible to use maternal whole blood as a sample for DNA extraction.

### Aspects of the Method:

New probe design for motif

Broader definition of motif

IT for identification of target sequences, primers with 3′-ends etc.

Use of defined common 3′-ends in multiplex PCR (e.g. −CAA in >50% or in 100% of all primers

1. A method for detecting and/or quantifying one or more target amplicons produced by amplification, wherein the detecting and/or quantifying is carried out during amplification or after an amplification endpoint has been reached, the method comprising:
preparing an amplification reaction mixture comprising:
sample nucleic acids;
at least one target-specific primer pair;
an optional probe, wherein at least one primer of the target-specific primer pair or the probe, if present, is labeled with a fluorescent dye; and
a fluorescent double-stranded DNA-binding dye, where fluorescence from the dye is capable of quenching fluorescent signal from the labeled primer or probe, if present;
subjecting the amplification mixture to amplification; and
detecting fluorescent signal to detect and/or quantify the one or more target amplicons.

2. A method for detecting and/or quantifying one or more target amplicons produced by amplification, wherein the detecting and/or quantifying is carried out during amplification or after an amplification endpoint has been reached, the method comprising:
preparing an amplification reaction mixture comprising:
sample nucleic acids;
at least one target-specific primer pair, wherein at least one primer in the target-specific primer pair comprises a nucleotide tag at the 5′ end of the primer;
at least one fluorescently labeled primer or probe that is capable of annealing to the nucleotide tag, directly or via one or more intervening primers, whereby the label can become linked to the nucleotide tag; and
a fluorescent double-stranded DNA-binding dye, where fluorescence from the dye is capable of quenching fluorescent signal from the labeled primer or probe;
subjecting the amplification mixture to amplification; and
detecting fluorescent signal to detect and/or quantify the one or more target amplicons.

3. The method of claim 2, wherein the fluorescence from the dye quenches fluorescent signal from the labeled primer or probe when the labeled primer or probe is incorporated into, or hybridized to, an amplification product.

4. A method for detecting an allele in a sample, the method comprising:
preparing an amplification mixture comprising:
sample nucleic acids;
two allele-specific primer pairs, wherein:
at least one primer in each primer pair is specific for an allele and is tagged with a distinct nucleotide tag at the 5′ end of the primer; and
the other primer in each pair can be the same or different from one another;
at least two differently fluorescently labeled primers or probes, each capable of annealing to one of the nucleotide tags, directly or via one or more intervening primers, whereby one label can become linked to one nucleotide tag and a different label can become linked to the other nucleotide tag;
subjecting the amplification mixture to amplification; and
detecting fluorescent signal to detect the allele in the sample.

5. The method of claim 4, wherein the amplification mixture additionally comprises a fluorescent double-stranded DNA-binding dye, where fluorescence from the dye is capable of quenching fluorescent signal from the labeled primers or probes.

6. The method of claim 5, wherein the fluorescence from the dye quenches fluorescent signal from the labeled primers or probes when the labeled primers or probes are incorporated into, or hybridized to, an amplification product.

7. The method of claim 4, wherein two differently labeled primers are employed, additionally comprising including in the reaction one or more quencher oligonucleotide(s) that comprise(s) a sequence that is capable of hybridizing to at least part of the nucleotide tag(s) and a fluorescence quencher, wherein hybridization to unincorporated fluorescently labeled primer(s) quenches the fluorescent label(s).

8. The method of claim 7, wherein the fluorescence quencher is at the 3′ end of the quencher oligonucleotide.

9. The method of claim 7, wherein the fluorescence quencher is attached to an internal nucleotide of the quencher oligonucleotide.

10. The method of claim 7, wherein the amplification mixture comprises at least two quencher oligonucleotides, one specific for each nucleotide tag.

11. A method for detecting an allele in a sample, the method comprising:
preparing an amplification mixture comprising:
sample nucleic acids;
two allele-specific oligonucleotides, wherein each oligonucleotide comprises a target-specific sequence linked to a distinct 3′ nucleotide tag; and
at least two differently fluorescently labeled primers or probes, each capable of annealing to one of the nucleotide tags, whereby one label can become linked to one nucleotide tag and a different label can become linked to the other nucleotide tag;
subjecting the amplification mixture to amplification; and
detecting fluorescent signal to detect the allele in the sample.

12. The method of claim 11, wherein two differently labeled primers are employed, additionally comprising including in the reaction one or more quencher oligonucleotide(s) that comprise(s) a sequence that is capable of hybridizing to at least part of the nucleotide tag(s) and a fluorescence quencher, wherein hybridization to unincorporated fluorescently labeled primer(s) quenches the fluorescent label(s).

13. The method of claim 12, wherein the fluorescence quencher is at the 3′ end of the quencher oligonucleotide.

14. The method of claim 12, wherein the fluorescence quencher is attached to an internal nucleotide of the quencher oligonucleotide.

15. The method of claim 12, wherein the amplification mixture comprises at least two quencher oligonucleotides, one specific for each nucleotide tag.

16. (canceled)

17. A method for tagging a plurality of target nucleic acids in a sample with common nucleotide tags, the method comprising:
contacting the sample with:
a plurality of 5′ oligonucleotides, one for each target nucleic acid, wherein each 5′ oligonucleotide comprises a first nucleotide tag that is linked, to and 5′ of, a target-specific sequence;
a plurality of 3′ oligonucleotides, one for each target nucleic acid, wherein each 3′ oligonucleotide comprises a target-specific sequence that is linked to, and 5′ of, a second nucleotide tag,
wherein the target-specific sequence of each 5′ oligonucleotide hybridizes to a target nucleic acid immediately adjacent to the target-specific sequence of the 3′ oligonucleotide, with an overlap such that one or more of the 5′-most base(s) of the 3′ oligonucleotide is/are displaced from the target nucleic acids, forming a flap;
a flap endonuclease; and
a ligase,
to produce a plurality of tagged target nucleic acids, each comprising the first and second tags.

18. (canceled)

19. A method for determining the methylation state of cytosine in a target nucleic acid sequence in a sample, the method comprising:
treating the sample to convert methylated cytosine(s) to uracil(s) in the target nucleic acids to produce a treated sample;
contacting the treated sample with:
a first 5′ oligonucleotide comprising a first nucleotide tag that is linked to, and 5′ of, a first melting temperature discriminator sequence that is linked to, and 5′ of, a 5′ target-specific sequence, wherein the 3′-most base is a G;
a first 3′ oligonucleotide comprising a G linked to a 3′ target-specific sequence,
wherein the target-specific sequence of the first 5′ oligonucleotide hybridizes to a target nucleic acid immediately adjacent to the target-specific sequence of the first 3′ oligonucleotide, with an overlap such that at least the G of the 3′ oligonucleotide is displaced from the target nucleic acids, forming a flap;
a second 5′ oligonucleotide comprising the same first nucleotide tag that is linked to, and 5′ of, a second melting temperature discriminator sequence that is linked to, and 5′ of, a 5′ target-specific sequence, wherein the 3′-most base is an A;
a second 3′ oligonucleotide comprising an A linked to the 3′ target-specific sequence;
wherein the target-specific sequence of the second 5′ oligonucleotide hybridizes to a target nucleic acid immediately adjacent to the target-specific sequence of the second 3′ oligonucleotide, with an overlap such that at least the A of the 3′ oligonucleotide is displaced from the target nucleic acids, forming a flap;
a flap endonuclease; and
a ligase
to produce a ligation product from the first 5′ and 3′ oligonucleotides if the target nucleic acid comprised a methylated cytosine or from the second 5′ and 3′ oligonucleotides if the target nucleic acids comprised an unmethylated cytosine.

20. (canceled)

21. (canceled)

22. A method for detecting a relative copy number difference in target nucleic acids in a sample, the method comprising:
subjecting a sample to preamplification using primers capable of amplifying a plurality of target nucleic acids to produce a plurality of target amplicons, so that the relative copy numbers of the target nucleic acids is substantially maintained, where some of the target nucleic acids are present on first chromosome and some of the target nucleic acids are present on a second, different chromosome;
determining the number of copies of target amplicons derived from the first chromosome and the number of copies of target amplicons derived from the second chromosome;
and determining the relative copy difference for the first and second chromosomes, wherein said method can detect a relative copy number difference less than 1.5.

23-27. (canceled)

28. A method for detecting a relative copy number difference between alleles at one or more target loci in a sample comprising a first allele and a second, different allele at least one target locus, the method comprising:
subjecting a sample to preamplification using primers capable of amplifying the first and second alleles to produce a plurality of target amplicons, so that the relative copy numbers of the first and second alleles is substantially maintained;
distributing the target amplicons into a plurality of amplification mixtures and carrying out digital amplification;
determining the number of amplification mixtures that contain a target amplicon derived from the first allele, and determining the number of amplification mixtures that contain a target amplicon derived from the second allele;
determining the ratio of amplification mixtures that contain the first allele to those that contain the second allele to detect the relative copy difference for the first and second alleles, wherein said method can detect a relative copy number difference less than 1.5.

29-34. (canceled)

35. A method for detecting fetal aneuploidy in a maternal bodily fluid sample from a pregnant subject, the method comprising:
subjecting a sample of a maternal bodily fluid sample, or a fraction thereof, to preamplification using primer pairs capable of amplifying at least a plurality of target nucleic acids to produce a plurality of target amplicons, so that the relative copy numbers of the target nucleic acids is substantially maintained, where some of the target nucleic acids are present on a first chromosome and some of the target nucleic acids are present on a second, different chromosome, wherein:
each primer employed for preamplification comprises a nucleotide tag, so that preamplification produces target amplicons comprising first a first nucleotide tag at one end and a second nucleotide tag a the other end, wherein all target amplicons derived from a given chromosome comprise the same first and second nucleotide tags; and
all target amplicons derived from a given chromosome are detectable with a common probe;
distributing the target amplicons into a plurality of amplification mixtures and carrying out multiplex digital amplification using:
a primer pair specific for the first and second nucleotide tags in target amplicons derived from the first chromosome;
a common probe specific for the target amplicons derived from the first chromosome;
a primer pair specific for the first and second nucleotide tags in target amplicons derived from the second chromosome; and
a common probe specific for the target amplicons derived from the second chromosome;
determining the number of amplification mixtures that contain a target amplicon derived from the first chromosome, and determining the number of amplification mixtures that contain a target amplicon derived from the second chromosome;
determining the ratio of amplification mixtures that contain the first chromosome to those that contain the second to detect the relative copy difference for the first and second alleles.

36-42. (canceled)

43. A method for detecting a relative copy number difference between at least two loci in genomic DNA or RNA in a sample, the method comprising:
quantifying the amount, in the sample, of a first non-coding RNA expressed from a chromosomal region linked to a first locus;
quantifying the amount, in the sample, of a second non-coding RNA expressed from a chromosomal region linked to a second locus;
determining a ratio of the amount of the first non-coding RNA to the amount of the second non-coding RNA, wherein a ratio significantly different from one indicates a copy number difference between the first and second locus.

44-46. (canceled)

47. A method for detecting a relative copy number difference between at least two loci in genomic DNA a sample, the method comprising:
producing, from the sample, a first DNA sequencing template that comprises, 5′ to 3′, a primer binding site for a forward DNA sequencing primer, linked directly, or via an intervening sequence, to a first target nucleotide sequence derived from the first locus, which is linked directly, or via an intervening sequence, to a primer binding site for a reverse DNA sequencing primer;
producing, from the sample, a second DNA sequencing template that comprises, 5′ to 3′, the primer binding site for the forward DNA sequencing primer, linked directly, or via an intervening sequence, to a second target nucleotide sequence derived from the second locus, which is linked directly, or via an intervening sequence, to a primer binding site for the reverse DNA sequencing primer, wherein:
the forward and reverse DNA sequencing primer binding sites are the same in both DNA sequencing templates; and
the first and second DNA sequencing templates are produced from the sample substantially in proportion to the copy number of the first and second loci in the sample;
determining the nucleotide sequences of the DNA sequencing templates;
quantifying the amount of first and second DNA sequencing templates;
determining a ratio of the amount of the first DNA sequencing template to the amount of the second DNA sequencing template to determine a copy number difference between the first and second locus.

48-54. (canceled)

55. A method for detecting and/or quantifying one or more fetal target nucleic acids in a maternal bodily fluid sample from a pregnant subject, the method comprising:
treating the sample to enrich for amplifiable fetal nucleic acids and produce a treated sample, wherein the treated sample comprises a higher percentage of fetal nucleic acids that are capable of being amplified, as compared to the percentage of maternal nucleic acids that are capable of being amplified;
amplifying the one or more fetal target nucleic acids; and
detecting and/or quantifying the one or more fetal target nucleic acids.

56-65. (canceled)
